(19) United States
(12) Patent Application Publication
(10) Pub. No.: US 2012/0074395 A1
(43) Pub. Date: Mar. 29, 2012

(54) ORGANIC ELECTROLUMINESCENT ELEMENT

(75) Inventors: Nobuhiro Yabunouchi, Chiba (JP); Takashi Arakane, Chiba (JP); Kazuki Nishimura, Chiba (JP); Chishio Hosokawa, Chiba (JP)

(73) Assignee: IDEMITSU KOSAN CO., LTD., Tokyo (JP)

(21) Appl. No.: 13/260,996

(22) PCT Filed: Mar. 31, 2010
(86) PCT No.: PCT/JP10/55844

§ 371 Date: Dec. 15, 2011

(30) Foreign Application Priority Data
Apr. 1, 2009 (JP) 2009-089468

(51) Int. Cl. H01L 51/54

(52) U.S. Cl. 257/40; 257/E51.026

(57) ABSTRACT

Provided is an organic EL device, including: an anode; a cathode; and an organic thin-film layer provided between the anode and the cathode, in which: the organic thin-film layer has a light emitting layer containing a host material and a light emitting material, and a hole transporting layer; and the hole transporting layer has a first hole transporting layer and a second hole transporting layer in the stated order from the anode; the first hole transporting layer contains a specific amine compound; and the second hole transporting layer contains a specific amine compound; or the hole transporting layer has a layer containing a specific electron acceptable compound and a first hole transporting layer; and the first hole-transporting layer contains a specific amine compound. The organic EL device has a reduced driving voltage, high luminous efficiency, and excellent practicality.

### TECHNICAL FIELD

The present invention relates to an organic electroluminescence device (which may hereinafter be referred to as “organic EL device”) using a specific compound in a hole transporting layer.

### BACKGROUND ART

A large number of organic EL devices each using an organic substance have been developed because of their potential to find applications in solid emission-type, inexpensive, large-area, full-color display devices. In general, an organic EL device is constructed of a light emitting layer and a pair of opposing electrodes between which the layer is interposed. Light emission is the following phenomenon. That is, upon application of an electric field to both electrodes, an electron is injected from a cathode side and a hole is injected from an anode side, and further, the electron recombines with the hole in the light emitting layer to produce an excited state, and energy generated upon return to a ground state from the excited state is radiated as light.

While organic EL devices of various forms have been known, there has been proposed, for example, such an organic EL device that an aromatic amine derivative having a specific substituent having a thiophene structure or an aromatic amine derivative having a carbazole skeleton to which a diarylamino group is bonded is used as a hole injecting material or a hole transporting material (see, for example, Patent Literatures 1 and 2).

### CITATION LIST

### Patent Literature

[PTL 1] WO 2008-023759 A1
[PTL 2] WO 2008-062636 A1

### SUMMARY OF INVENTION

### Technical Problem

However, such organic EL device as described above has caused an increase in its driving voltage in some cases because charge transfer between molecules having different molecular structures in the above-mentioned material may not progress smoothly.

In view of the foregoing, an object of the present invention is to provide an organic EL device having a reduced driving voltage, a long lifetime, and excellent practicality.

### Solution to Problem

The inventors of the present invention have made extensive studies to achieve the object, and as a result, have found that an organic EL device having a low driving voltage and a long lifetime can be produced as described below. A compound having a specific diamine structure is used as a material for a first hole transporting layer, and an aromatic amine derivative having a dibenzofuran structure and a carbazole structure is used as a material for a second hole transporting layer. Alternatively, a specific electron acceptable compound is used, and an aromatic amine derivative having a dibenzofuran structure and a carbazole structure is used as a material for a first hole transporting layer. Thus, the inventors have completed the present invention.

That is, a first invention of the present application is an organic electroluminescence device, including: an anode; a cathode; and an organic thin-film layer provided between the anode and the cathode,

in which: the organic thin-film layer has a light emitting layer containing a host material and a light emitting material, and a hole transporting layer provided on a side closer to the anode than the light emitting layer; the hole transporting layer has a first hole transporting layer and a second hole transporting layer in the stated order from the anode; the first hole transporting layer contains a compound represented by the following general formula (1); and the second hole transporting layer contains a compound represented by the following general formula (2):

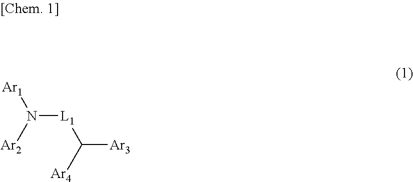

where L₁ represents a substituted or unsubstituted arylene group having 10 to 40 ring carbon atoms, and Ar₁ to Ar₄ each represent a substituted or unsubstituted aryl group having 6 to 60 ring carbon atoms, or a heteroaryl group having 6 to 60 ring atoms;

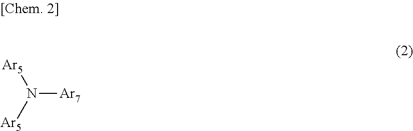

where at least one of Ar₅ to Ar₇ represents a group represented by the following general formula (3), and a group represented by any one of Ar₅ to Ar₇ except the group represented by the general formula (3) is a group represented by the following general formula (4) or (5), or a substituted or unsubstituted aryl group having 6 to 40 carbon atoms;

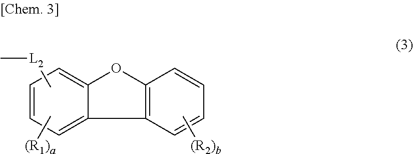

where: L₂ represents a single bond, or a substituted or unsubstituted arylene group having 6 to 50 ring carbon atoms, and a substituent which L₂ may have is a linear or branched alkyl group having 1 to 10 carbon atoms, a cycloalkyl group having 3 to 10 ring carbon atoms, a trialkylsilyl group having 3 to 10 carbon atoms, a triarylsilyl group having 18 to 30 ring carbon atoms, an alkylarylsilyl group having 8 to 15 carbon atoms whose aryl moiety has 6 to 14 ring carbon atoms, an aryl group having 6 to 14 ring carbon atoms, a halogen atom, or a cyano group; and

R₁ and R₂ each independently represent a substituted or unsubstituted, linear or branched alkyl group having 1 to 10 carbon atoms, a substituted or unsubstituted cycloalkyl group having 3 to 10 ring carbon atoms, a substituted or unsubstituted trialkylsilyl group having 3 to 10 carbon atoms, a substituted or unsubstituted triarylsilyl group having 18 to 30 ring carbon atoms, a substituted or unsubstituted alkylarylsilyl group having 8 to 15 carbon atoms whose aryl moiety has 6 to 14 ring carbon atoms, a substituted or unsubstituted aryl group having 6 to 16 ring carbon atoms, a halogen atom, or a cyano group, and a plurality of adjacent R₁'s or R₂'s may be bonded to each other to form a saturated or unsaturated, divalent group that forms a ring, and a and b each independently represent an integer of 0 to 4;

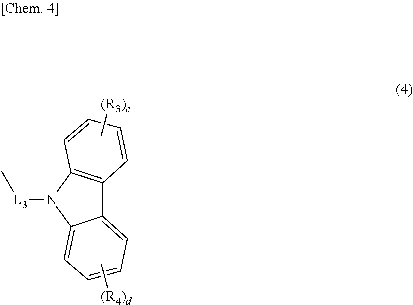

where: L₃ represents a single bond, or a substituted or unsubstituted arylene group having 6 to 50 ring carbon atoms, and a substituent which L₃ may have is a linear or branched alkyl group having 1 to 10 carbon atoms, a cycloalkyl group having 3 to 10 ring carbon atoms, a trialkylsilyl group having 3 to 10 carbon atoms, a triarylsilyl group having 18 to 30 ring carbon atoms, an alkylarylsilyl group having 8 to 15 carbon atoms whose aryl moiety has 6 to 14 ring carbon atoms, an aryl group having 6 to 14 ring carbon atoms, a halogen atom, or a cyano group;

c and d each independently represent an integer of 0 to 4; and

R₃ and R₄ each independently represent a substituted or unsubstituted, linear or branched alkyl group having 1 to 10 carbon atoms, a substituted or unsubstituted cycloalkyl group having 3 to 10 ring carbon atoms, a substituted or unsubstituted trialkylsilyl group having 3 to 10 carbon atoms, a substituted or unsubstituted triarylsilyl group having 18 to 30 ring carbon atoms, a substituted or unsubstituted alkylarylsilyl group having 8 to 15 carbon atoms whose aryl moiety has 6 to 14 ring carbon atoms, a substituted or unsubstituted aryl group having 6 to 14 ring carbon atoms, a halogen atom, or a cyano group, and a plurality of adjacent R₃'s or R₄'s may be bonded to each other to form a saturated or unsaturated, divalent group that forms a ring; and

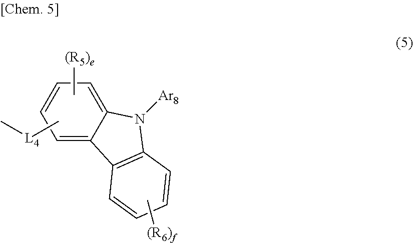

where: L₄ represents a single bond, or a substituted or unsubstituted arylene group having 6 to 50 ring carbon atoms, and a substituent which L₄ may have is a linear or branched alkyl group having 1 to 10 carbon atoms, a cycloalkyl group having 3 to 10 ring carbon atoms, a trialkylsilyl group having 3 to 10 carbon atoms, a triarylsilyl group having 18 to 30 ring carbon atoms, an alkylarylsilyl group having 8 to 15 carbon atoms whose aryl moiety has 6 to 14 ring carbon atoms, an aryl group having 6 to 14 ring carbon atoms, a halogen atom, or a cyano group;

Ar₈ represents a substituted or unsubstituted aryl group having 6 to 14 ring carbon atoms, and a substituent which Ar₈ may have is a linear or branched alkyl group having 1 to 10 carbon atoms, a cycloalkyl group having 3 to 10 ring carbon atoms, a trialkylsilyl group having 3 to 10 carbon atoms, a triarylsilyl group having 18 to 30 ring carbon atoms, an alkylarylsilyl group having 8 to 15 carbon atoms whose aryl moiety has 6 to 14 ring carbon atoms, an aryl group having 6 to 14 ring carbon atoms, a halogen atom, or a cyano group;

e represents an integer of 0 to 3 and f represents an integer of 0 to 4; and

R₅ and R₆ each independently represent a substituted or unsubstituted, linear or branched alkyl group having 1 to 10 carbon atoms, a substituted or unsubstituted cycloalkyl group having 3 to 10 ring carbon atoms, a substituted or unsubstituted trialkylsilyl group having 3 to 10 carbon atoms, a substituted or unsubstituted triarylsilyl group having 18 to 30 ring carbon atoms, a substituted or unsubstituted alkylarylsilyl group having 8 to 15 carbon atoms whose aryl moiety has 6 to 14 ring carbon atoms, a substituted or unsubstituted aryl group having 6 to 14 ring carbon atoms, a halogen atom, or a cyano group, and a plurality of adjacent R₅'s or R₆'s may be bonded to each other to form a saturated or unsaturated, divalent group that forms a ring.

Further, a second invention of the present application is an organic electroluminescence device, including: an anode; a cathode; and an organic thin-film layer provided between the anode and the cathode,

in which: the organic thin-film layer has a light emitting layer containing a host material and a light emitting material, and a hole transporting layer provided on a side closer to the anode than the light emitting layer; the hole transporting layer has a layer containing an electron acceptable compound and a first hole transporting layer in the stated order from the anode; the electron acceptable compound is represented by the following general formula (10); and the first hole transporting layer contains a compound represented by the above-mentioned general formula (2):

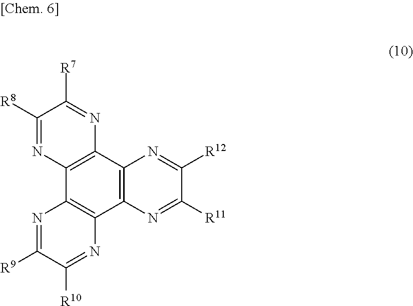

in the above-mentioned general formula (10), R⁷ to R¹² each independently represent a cyano group, —CONH₂, a carboxyl group, or —COOR¹³ where R¹³ represents an alkyl group having 1 to 20 carbon atoms, or R⁷ and R⁸, R⁹ and R¹⁰, or R¹¹ and R¹² are bonded to each other to represent a group represented by —CO—O—CO—.

The organic EL device of the present invention is applicable to an organic EL device that constructs any one of the red, green, and blue pixels needed for a full-color display as well because the device can suitably transport charge. In addition, the device can be expected to achieve the commonality of materials except the host material and the light emitting material in the light emitting layer. Accordingly, the reduction of a production cost for the device is expected.

### Advantageous Effects of Invention

According to the present invention, there can be provided an organic EL device having a reduced driving voltage, a long lifetime, and excellent practicality.

### BRIEF DESCRIPTION OF DRAWING

FIG. 1 is a view illustrating the schematic construction of an embodiment of an organic EL device of the present invention.

### REFERENCE SIGNS LIST

1: organic EL device
2: substrate
3: anode
4: cathode
5: light emitting layer
6: hole transporting layer
61: first hole transporting layer
62: second hole transporting layer
7: electron injecting/transporting layer
10: organic thin-film layer

### DESCRIPTION OF EMBODIMENTS

An organic EL device of a first invention of the present application includes an anode a cathode and an organic thin-film layer provided between the anode and the cathode. The organic thin-film layer has a light emitting layer containing a host material and a light emitting material, and a hole transporting layer provided on a side closer to the anode than the light emitting layer. In addition, the hole transporting layer has a first hole transporting layer and a second hole transporting layer in the stated order from the anode, the first hole transporting layer contains a compound represented by the following general formula (1), and the second hole transporting layer contains a compound represented by the following general formula (2).

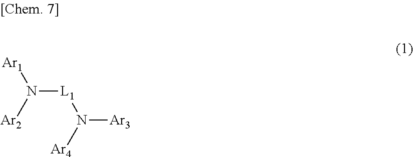

(In the formula, L₁ represents a substituted or unsubstituted arylene group having 10 to 40 ring carbon atoms, and Ar₁ to Ar₄ each represent a substituted or unsubstituted aryl group having 6 to 60 ring carbon atoms, or a heteroaryl group having 6 to 60 ring atoms.)

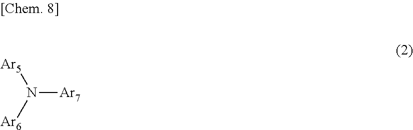

(In the formula, at least one of Ar₅ to Ar₇ represents a group represented by the following general formula (3), and a group represented by any one of Ar₅ to Ar₇ except the group represented by the general formula (3) is a group represented by the following general formula (4) or (5), or a substituted or unsubstituted aryl group having 6 to 40 carbon atoms.)

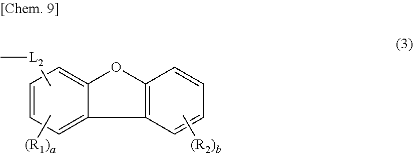

(In the formula: L₂ represents a single bond, or a substituted or unsubstituted arylene group having 6 to 50 ring carbon atoms, and a substituent which L₂ may have is a linear or branched alkyl group having 1 to 10 carbon atoms, a cycloalkyl group having 3 to 10 ring carbon atoms, a trialkylsilyl group having 3 to 10 carbon atoms, a triarylsilyl group having 18 to 30 ring carbon atoms, an alkylarylsilyl group having 8 to 15 carbon atoms whose aryl moiety has 6 to 14 ring carbon atoms, an aryl group having 6 to 14 ring carbon atoms, a halogen atom, or a cyano group; and

R₁ and R₂ each independently represent a substituted or unsubstituted, linear or branched alkyl group having 1 to 10 carbon atoms, a substituted or unsubstituted cycloalkyl group having 3 to 10 ring carbon atoms, a substituted or unsubstituted trialkylsilyl group having 3 to 10 carbon atoms, a substituted or unsubstituted triarylsilyl group having 18 to 30 ring carbon atoms, a substituted or unsubstituted alkylarylsilyl group having 8 to 15 carbon atoms (whose aryl moiety has 6 to 14 ring carbon atoms), a substituted or unsubstituted aryl group having 6 to 16 ring carbon atoms, a halogen atom, or a cyano group, and a plurality of adjacent R₁'s or R₂'s may be bonded to each other to form a saturated or unsaturated, divalent group that forms a ring, and a and b each independently represent an integer of 0 to 4.)

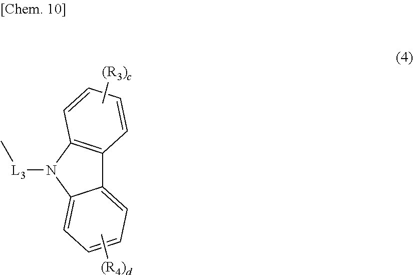

(In the formula: L₃ represents a single bond, or a substituted or unsubstituted arylene group having 6 to 50 ring carbon atoms, and a substituent which L₃ may have is a linear or branched alkyl group having 1 to 10 carbon atoms, a cycloalkyl group having 3 to 10 ring carbon atoms, a trialkylsilyl group having 3 to 10 carbon atoms, a triarylsilyl group having 18 to 30 ring carbon atoms, an alkylarylsilyl group having 8 to 15 carbon atoms (whose aryl moiety has 6 to 14 ring carbon atoms), an aryl group having 6 to 14 ring carbon atoms, a halogen atom, or a cyano group;

c and d each independently represent an integer of 0 to 4; and

R₃ and R₄ each independently represent a substituted or unsubstituted, linear or branched alkyl group having 1 to 10 carbon atoms, a substituted or unsubstituted cycloalkyl group having 3 to 10 ring carbon atoms, a substituted or unsubstituted trialkylsilyl group having 3 to 10 carbon atoms, a substituted or unsubstituted triarylsilyl group having 18 to 30 ring carbon atoms, a substituted or unsubstituted alkylarylsilyl group having 8 to 15 carbon atoms (whose aryl moiety has 6 to 14 ring carbon atoms), a substituted or unsubstituted aryl group having 6 to 14 ring carbon atoms, a halogen atom, or a cyano group, and a plurality of adjacent R₃'s or R₄'s may be bonded to each other to form a saturated or unsaturated, divalent group that forms a ring.)

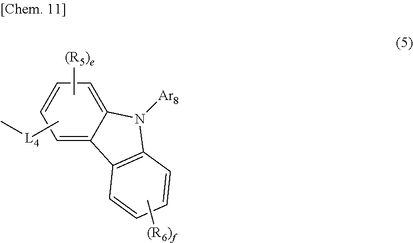

(In the formula: L₄ represents a single bond, or a substituted or unsubstituted arylene group having 6 to 50 ring carbon atoms, and a substituent which L₄ may have is a linear or branched alkyl group having 1 to 10 carbon atoms, a cycloalkyl group having 3 to 10 ring carbon atoms, a trialkylsilyl group having 3 to 10 carbon atoms, a triarylsilyl group having 18 to 30 ring carbon atoms, an alkylarylsilyl group having 8 to 15 carbon atoms (whose aryl moiety has 6 to 14 ring carbon atoms), an aryl group having 6 to 14 ring carbon atoms, a halogen atom, or a cyano group;

Ar₈ represents a substituted or unsubstituted aryl group having 6 to 14 ring carbon atoms, and a substituent which Ar₈ may have is a linear or branched alkyl group having 1 to 10 carbon atoms, a cycloalkyl group having 3 to 10 ring carbon atoms, a trialkylsilyl group having 3 to 10 carbon atoms, a triarylsilyl group having 18 to 30 ring carbon atoms, an alkylarylsilyl group having 8 to 15 carbon atoms (whose aryl moiety has 6 to 14 ring carbon atoms), an aryl group having 6 to 14 ring carbon atoms, a halogen atom, or a cyano group;

e represents an integer of 0 to 3 and f represents an integer of 0 to 4; and

R₅ and R₆ each independently represent a substituted or unsubstituted, linear or branched alkyl group having 1 to 10 carbon atoms, a substituted or unsubstituted cycloalkyl group having 3 to 10 ring carbon atoms, a substituted or unsubstituted trialkylsilyl group having 3 to 10 carbon atoms, a substituted or unsubstituted triarylsilyl group having 18 to 30 ring carbon atoms, a substituted or unsubstituted alkylarylsilyl group having 8 to 15 carbon atoms (whose aryl moiety has 6 to 14 ring carbon atoms), a substituted or unsubstituted aryl group having 6 to 14 ring carbon atoms, a halogen atom, or a cyano group, and a plurality of adjacent R₅'s or R₆'s may be bonded to each other to form a saturated or unsaturated, divalent group that forms a ring.)

The case where L₂ in the general formula (3) and L₄ in the general formula (5) each represent an arylene group is preferred because an increase in the electron density of the compound represented by the general formula (2) is suppressed and its IP increases, and hence the driving voltage of the device easily becomes low. Further, when a dibenzofuran structure or a carbazole structure is bonded to a nitrogen atom through an arylene group, it becomes hard to oxidize an amine, and hence the compound becomes stable in many cases. As a result, the lifetime of the device easily lengthens. In addition, when L₄ in the general formula (5) represents an arylene group, the compound can be easily synthesized because the compound is stable.

In addition, when the arylene group represented by L₄ in the general formula (5) is represented by the following general formula (8), an increase in the electron density of the amine compound is suppressed. As a result, its IP increases, the property by which a hole is injected into the light emitting layer is improved, and a reduction in the driving voltage of the device can be expected. In particular, when the compound represented by the general formula (2) has a dibenzofuran structure-containing group represented by the general formula (3) and a carbazole structure-containing group represented by the general formula (4) and/or the general formula (5), it is preferred that L₃ and L₄ in the general formulae (4) and (5) be each independently represented by the following general formula (8).

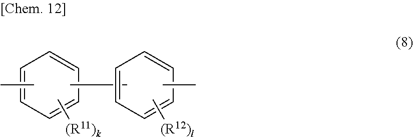

[R¹¹ and R¹² each independently represent a linear or branched alkyl group having 1 to 10 carbon atoms, a cycloalkyl group having 3 to 10 ring carbon atoms, a trialkylsilyl group having 3 to 10 carbon atoms, a triarylsilyl group having 18 to 30 ring carbon atoms, an alkylarylsilyl group having 8 to 15 carbon atoms (whose aryl moiety has 6 to 14 ring carbon atoms), an aryl group having 6 to 16 ring carbon atoms, a halogen atom, or a cyano group, and a plurality of adjacent R¹¹'s or R¹²'s may be bonded to form a saturated or unsaturated ring.

k and l each independently represent an integer of 0 to 4.]

The compounds represented by the formulae (1) and (2) can each be suitably used in a hole transporting layer because the compounds have hole injecting/transporting properties.

In addition, the compounds represented by the formulae (1) and (2) each have a small affinity level Af. Accordingly, excellent electron blocking property is exerted by forming the hole transporting layer joined to the light emitting layer with those compounds.

Moreover, the compounds represented by the formulae (1) and (2) each have high electron resistance. Accordingly, the lifetime of the organic EL device hardly reduces even by the concentration of electrons at the time of electron blocking.

The hole transporting layer of the organic EL device of the first invention of the present application is formed by using such compounds represented by the formulae (1) and (2). Accordingly, a hole can be injected into the light emitting layer while an electron is trapped in the light emitting layer. As a result, the probability of charge recombination is increased, and hence high-efficiency light emission can be obtained. The performance improvement, which is effective irrespective of whether the device emits fluorescence or phosphorescence, is particularly effective for the phosphorescence.

In addition, electrons concentrate on an interface between the light emitting layer and the hole transporting layer upon electron blocking. However, the compounds represented by the formulae (1) and (2) each have high electron resistance, and hence an emission lifetime hardly reduces.

In addition, the compounds represented by the formulae (1) and (2) each show the following tendency. The compounds each have so small an Af that an electron hardly enters and its transfer toward the anode is trapped.

Further, the lifetime of the entire device can be lengthened by trapping an electron in the compound represented by the formula (2) having larger electron resistance than that of the compound represented by the formula (1).

The compound represented by the formula (2) is a monoamine-based compound and has a dibenzofuran group, and its Eg is larger than that of the compound represented by the formula (1). Accordingly, the compound generally has a smaller Af than that of the compound represented by the formula (1), and hence hardly allows the transfer of an electron from the light emitting layer toward the anode. The above-mentioned effect is significant in a phosphorescent light emitting layer because of the following reason. As the Eg of the light emitting layer is large, its Af is generally small, and hence the difficulty with which an electron is transferred toward the anode is raised.

In addition, the steric bulkiness of dibenzofuran exerts such a steric effect that a distance to a molecule in the adjacent first hole transporting layer is lengthened. Accordingly, a carrier trap is formed at an interface between the second hole transporting layer and the first hole transporting layer. Accordingly, the lifetime of the entire device can be lengthened by trapping an electron transferring from the cathode side in the compound represented by the formula (2) having larger electron resistance than that of the compound represented by the formula (1).

It should be noted that the affinity level Af (electron affinity) refers to energy to be discharged or absorbed when a molecule of a material is provided with one electron, and the energy is defined as being positive when discharged or as being negative when absorbed.

The affinity level Af is specified by an ionization potential Ip and an optical energy gap Eg(S) as described below.

Af=Ip−Eg(S)

Here, the ionization potential Ip means energy needed for removing an electron from the compound of each material to ionize the compound, and is a value measured with, for example, an ultraviolet photoelectron spectrometer (AC-3, Riken Keiki Co., Ltd.).

The optical energy gap Eg(S) refers to a difference between a conduction level and a valence level, and is determined by, for example, converting a wavelength value for a point of intersection of the tangent of the absorption spectrum of a toluene dilute solution of each material at longer wavelengths and a baseline (zero absorption) into energy.

Further, each of the compounds represented by the formulae (1) and (2) has a high glass transition temperature (Tg) and is excellent in heat resistance. In particular, the introduction of a substituent having a large molecular weight can improve the heat resistance of the hole transporting layer.

Here, α-NPD (see, for example, US 2006-0088728 A1), which has been conventionally used as a material that forms a hole transporting layer, has been poor in heat resistance because its Tg is 100° C. or less.

In contrast, in the present invention, the heat resistance of the organic EL device can be improved by adopting the compounds represented by the formulae (1) and (2) each having a high Tg.

In addition, in the invention of US 2006-0088728 A1, a hole injecting layer is formed by using a copper phthalocyanine compound.

However, the copper complex compound is not preferred because of the following reason. As the compound has absorption in a visible region, the compound takes on a blue tinge when turned into a thick film. In addition, a large number of limitations are imposed on the building of a device construction from the copper complex compound because of the following reason. As the compound has low amorphous property and high crystallinity, it is hard to turn the compound into a thick film.

In contrast, the compounds represented by the formulae (1) and (2) are suitable for being turned into thick films because each of the compounds has no large absorption in the visible region, has high amorphous property, and has low crystallinity.

Accordingly, various device constructions can be built in the organic EL device of the present invention adopting the compounds represented by the formulae (1) and (2).

The hole transporting layer in the organic electroluminescence device of the present invention is provided on a side closer to the anode than the light emitting layer, and serves to inject a hole from the anode into the light emitting layer.

The first hole transporting layer and the second hole transporting layer in the organic electroluminescence device of the present invention are each a layer functioning as a hole transporting layer that injects a hole into the light emitting layer. The layer provided on the anode side is referred to as “first hole transporting layer” and the layer provided on the light emitting layer side is referred to as “second hole transporting layer.”

In general, a plurality of hole transporting layers are provided for injecting holes from the anode into the highest occupied molecular orbital (HOMO) of the light emitting layer at a low voltage, and materials for the hole transporting layers are selected in such a manner that the HOMO levels of the hole transporting layers are caused to gradually approach the HOMO level of the light emitting layer in the direction from the hole transporting layer positioned on the anode side to the hole transporting layer positioned on the light emitting layer side.

In addition, the following has been known. When a material having a small affinity level is selected for the hole transporting layer adjacent to the light emitting layer in order that the probability of recombination between an electron and a hole in the light emitting layer may be increased, an electron coming from the cathode side can be trapped in the light emitting layer, which enables an improvement in luminous efficiency and the lengthening of the lifetime.

Accordingly, the ionization potential of the first hole transporting layer is preferably smaller than the ionization potential of the second hole transporting layer. Further, the difference is preferably 1.0 eV or less, more preferably 0.4 eV or less.

In addition, the affinity level of the first hole transporting layer is preferably smaller than the affinity level of the light emitting layer contacting the layer. Further, the difference is preferably 1.0 eV or less, more preferably 0.4 eV or less.

The case where the thickness of the above-mentioned first hole transporting layer is 10 to 200 nm is preferred, the case where the thickness is 15 to 150 nm is more preferred, and the case where the thickness is 20 to 100 nm is particularly preferred. In addition, the case where the thickness of the above-mentioned second hole transporting layer is 10 to 200 nm is preferred, the case where the thickness is 15 to 150 nm is more preferred, and the case where the thickness is 20 to 100 nm is particularly preferred.

The organic electroluminescence device of the present invention is preferably such that L₃ and L₄ in the general formula (4) and the general formula (5) each independently represent a phenylene group, a biphenyldiyl group, a terphenyldiyl group, a naphthylene group, or a phenanthrenediyl group.

The organic EL device of the present invention has the compound represented by the above-mentioned general formula (1) in the first hole transporting layer. As the compound has a large ionization potential, the transfer of a hole toward the second hole transporting layer is facilitated, and hence the driving voltage of the organic EL device to be obtained is reduced.

The compound represented by the general formula (1) preferably further satisfies the following conditions (2) to (6).

(2) The compound represented by the general formula (1) is asymmetric with respect to L₁.

The compound shows a small intermolecular interaction as compared with that of a compound symmetric with respect to L₁. Accordingly, its crystallization is suppressed and the yield in which the organic EL device is produced is improved. In addition, the compound is excellent in amorphous property, and hence adhesiveness at an interface with ITO or an organic layer adjacent to the first hole transporting layer is improved and the device is stabilized.

(3) L₁ in the general formula (1) represents a biphenyldiyl group.

In a cation state in which a hole is injected, the compound has an electrically stable quinoid structure and has excellent stability against oxidation.

(4) Ar₁ to Ar₄ in the general formula (1) each independently represent a substituted or unsubstituted phenyl group, a substituted or unsubstituted biphenylyl group, a substituted or unsubstituted terphenylyl group, or a substituted or unsubstituted phenanthryl group, or are each independently represented by the following general formula (6).

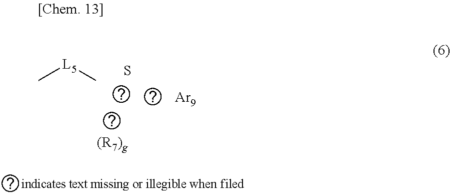

(In the formula: L₅ represents a substituted or unsubstituted arylene group having 6 to 50 ring carbon atoms, and a substituent which L₅ may have is a linear or branched alkyl group having 1 to 10 carbon atoms, a cycloalkyl group having 3 to 10 ring carbon atoms, a trialkylsilyl group having 3 to 10 carbon atoms, a triarylsilyl group having 18 to 30 ring carbon atoms, an alkylarylsilyl group having 8 to 15 carbon atoms (whose aryl moiety has 6 to 14 ring carbon atoms), an aryl group having 6 to 14 ring carbon atoms, a halogen atom, or a cyano group;

Ar₉ represents a substituted or unsubstituted aryl group having 6 to 14 ring carbon atoms, and a substituent which Ar₉ may have is a linear or branched alkyl group having 1 to 10 carbon atoms, a cycloalkyl group having 3 to 10 ring carbon atoms, a trialkylsilyl group having 3 to 10 carbon atoms, a triarylsilyl group having 18 to 30 ring carbon atoms, an alkylarylsilyl group having 8 to 15 carbon atoms (whose aryl moiety has 6 to 14 ring carbon atoms), an aryl group having 6 to 14 ring carbon atoms, a halogen atom, or a cyano group;

g represents 1 or 2; and

R₇ represents a substituted or unsubstituted, linear or branched alkyl group having 1 to 10 carbon atoms, a substituted or unsubstituted cycloalkyl group having 3 to 10 ring carbon atoms, a substituted or unsubstituted trialkylsilyl group having 3 to 10 carbon atoms, a substituted or unsubstituted triarylsilyl group having 18 to 30 ring carbon atoms, a substituted or unsubstituted alkylarylsilyl group having 8 to 15 carbon atoms (whose aryl moiety has 6 to 14 ring carbon atoms), a substituted or unsubstituted aryl group having 6 to 14 ring carbon atoms, a halogen atom, or a cyano group, and a plurality of R₇'s may be bonded to each other to form a saturated or unsaturated, divalent group that forms a ring.)

The structure represented by the above-mentioned general formula (6) is excellent in adhesiveness with ITO by virtue of an interaction between a lone pair and ITO. Accordingly, the structure has good hole injecting property, is hardly affected by the nature of ITO, and can have stable device performance.

(5) Ar₁ to Ar₄ in the general formula (1) each independently represent a substituted or unsubstituted phenyl group, a substituted or unsubstituted biphenyl group, a substituted or unsubstituted terphenyl group, or a substituted or unsubstituted phenanthryl group.

A phenyl group, a biphenylyl group, a terphenylyl group, and a phenanthryl group are a group of substituents each having excellent stability against both oxidation and reduction, and are each suitable as a substituent to be bonded to an amine.

(6) At least one of Ar₁ to Ar₄ in the general formula (1) is represented by the general formula (6).

The amine unit shows a small intermolecular interaction because of the presence of steric hindrance. Accordingly, its crystallization is suppressed and the yield in which the organic EL device is produced can be improved. In addition, an amine compound having an aryl group having a dibenzofuran structure and an aryl group having a carbazole structure has a large Eg, and can effectively block an electron from the light emitting layer, thereby improving the efficiency. In addition, the compound has a lifetime-lengthening effect because the compound suppresses the injection of an electron into the hole transporting layer. In particular, the combination of the compound with a blue light emitting device exerts a significant lifetime-lengthening effect.

The compound represented by the general formula (2) preferably further satisfies the following conditions (7) to (25).

(7) At least one of Ar₅ to Ar₇ in the general formula (2) represents a group represented by the general formula (4) or (5).
(8) At least one of Ar₅ to Ar₇ in the general formula (2) represents a group represented by the general formula (4).

It is assumed that the instability of carbazole against reduction is alleviated by an interaction between the N atom of carbazole and the N atom of an amine. The alleviation is preferred because the lifetime lengthens as a result thereof.

(9) At least one of Ar₅ to Ar₇ in the general formula (2) represents a group represented by the general formula (5).
(10) At least one of Ar₅ to Ar₇ in the general formula (2) is represented by the following general formula (7).

The amine unit shows a small intermolecular interaction because of the presence of steric hindrance. Accordingly, its crystallization is suppressed and the yield in which the organic EL device is produced is improved. In addition, the unit has a terphenyl group excellent in reduction stability. Accordingly, the reduction stability of a molecule of the unit is improved and the unit has a lengthening effect on the lifetime of the organic EL device to be obtained. In particular, the combination of the unit with a blue light emitting device exerts a significant lifetime-lengthening effect.

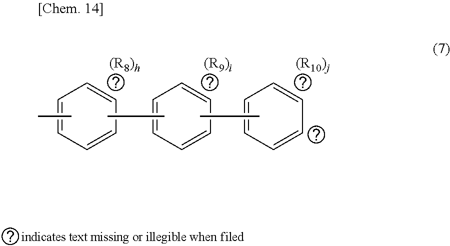

(In the formula, R₈ to R₁₀ each independently represent a substituted or unsubstituted, linear or branched alkyl group having 1 to 10 carbon atoms, a substituted or unsubstituted cycloalkyl group having to 10 ring carbon atoms, a substituted or unsubstituted trialkylsilyl group having 3 to 10 carbon atoms, a substituted or unsubstituted triarylsilyl group having 18 to 30 ring carbon atoms, a substituted or unsubstituted alkylarylsilyl group having 8 to 15 carbon atoms (whose aryl moiety has 6 to 14 ring carbon atoms), a substituted or unsubstituted aryl group having 6 to 16 ring carbon atoms, a halogen atom, or a cyano group, and a plurality of adjacent R₈'s, R₉'s, or R₁₀'s may be bonded to each other to form a saturated or unsaturated, divalent group that forms a ring, and h, i, and j each independently represent an integer of 0 to 4.)
(11) At least two of Ar₅ to Ar₇ in the general formula (2) are each represented by the general formula (3); when two of Ar₅ to Ar₇ are each represented by the general formula (3), the two groups each represented by the general formula (3) are different from each other; and when three of Ar₅ to Ar₇ represent groups each represented by the general formula (3), the three groups each represented by the general formula (2) are different from one another.

The molecular symmetry of an amine derivative having at least two kinds of aryl groups each having a dibenzofuran structure can be reduced. Accordingly, its crystallization is suppressed and the yield in which the organic EL device is produced can be improved. In addition, an amine compound having an aryl group having a dibenzofuran structure has a large Eg and can effectively block an electron from the light emitting layer, thereby improving the efficiency. In addition, the compound has a lifetime-lengthening effect because the compound suppresses the injection of an electron into the hole transporting layer. In particular, the combination of the compound with a blue light emitting device exerts a significant lifetime-lengthening effect.

In addition, it is assumed that the Ip reduces and hence a hole can be directly injected into a dopant in the host with ease. The foregoing is preferred because the driving voltage reduces as a result thereof.

(12) Ar₅ in the general formula (2) is represented by the general formula (3).
(13) In the general formula (2), Ar₅ is represented by the general formula (3) and Ar₆ and Ar₇ each represent a substituted or unsubstituted aryl group having 6 to 40 carbon atoms.
(14) In the general formula (2), Ar₅ is represented by the general formula (3), Ar₆ is represented by the general formula (3), and Ar₇ represents a substituted or unsubstituted aryl group having 6 to 40 carbon atoms.
(15) In the general formula (2), Ar₅ is represented by the general formula (3) and Ar₆ and Ar₇ are each represented by the general formula (4).
(16) In the general formula (2), Ar₅ is represented by the general formula (3), Ar₆ is represented by the general formula (3), and Ar₇ is represented by the general formula (4).
(17) In the general formula (2), Ar₅ is represented by the general formula (3) and Ar₆ and Ar₇ are each represented by the general formula (5).
(18) In the general formula (2), Ar₅ is represented by the general formula (3), Ar₆ is represented by the general formula (3), and Ar₇ is represented by the general formula (5).
(19) In the general formula (2), Ar₅ is represented by the general formula (3) and Ar₆ and Ar₇ are each represented by the general formula (7).
(20) In the general formula (2), Ar₅ is represented by the general formula (3), Ar₆ is represented by the general formula (3), and Ar₇ is represented by the general formula (7).
(21) In the general formula (2), Ar₅ is represented by the general formula (3), Ar₆ is represented by the general formula (4), and Ar₇ is represented by the general formula (5).
(22) In the general formula (2), Ar₅ is represented by the general formula (3), Ar₆ is represented by the general formula (4), and Ar₇ is represented by the general formula (7).
(23) In the general formula (2), Ar₅ is represented by the general formula (3), Ar₆ is represented by the general formula (4), and Ar₇ represents a substituted or unsubstituted aryl group having 6 to 40 carbon atoms.
(24) In the general formula (2), Ar₅ is represented by the general formula (3), Ar₆ is represented by the general formula (5), and Ar₇ represents a substituted or unsubstituted aryl group having 6 to 40 carbon atoms.
(25) In the general formula (2), Ar₅ is represented by the general formula (3), Ar₆ is represented by the general formula (7), and Ar₇ represents a substituted or unsubstituted aryl group having 6 to 40 carbon atoms.

In the general formulae (1) to (7), specific examples of the substituted or unsubstituted alkyl group represented by each of R₁ to R₁₀ include a methyl group, an ethyl group, a propyl group, an isopropyl group, an n-butyl group, an isobutyl group, a sec-butyl group, a tert-butyl group, an n-pentyl group, an n-hexyl group, an n-heptyl group, an n-octyl group, a hydroxymethyl group, a 1-hydroxyethyl group, a 2-hydroxyethyl group, a 2-hydroxyisobutyl group, a 1,2-dihydroxyethyl group, a 1,3-dihydroxyisopropyl group, a 2,3-dihydroxy-t-butyl group, and a 1,2,3-trihydroxypropyl group. Of those, a methyl group, an ethyl group, a propyl group, an isopropyl group, an n-butyl group, an isobutyl group, a sec-butyl group, and a tert-butyl group are preferred.

In the general formulae (1) to (7), specific examples of the substituted or unsubstituted cycloalkyl group represented by each of R₁ to R₁₀ include a cyclopropyl group, a cyclobutyl group, a cyclopentyl group, a cyclohexyl group, a cyclopentylmethyl group, a cyclohexylmethyl group, a cyclohexylethyl group, a 4-fluorocyclohexyl group, a 1-adamantyl group, a 2-adamantyl group, a 1-norbornyl group, and a 2-norbornyl group. Of those, a cyclopentyl group and a cyclohexyl group are preferred.

In the general formulae (1) to (7), specific examples of the trialkylsilyl group represented by each of R₁ to R₁₀ include a trimethylsilyl group, a vinyldimethylsilyl group, a triethylsilyl group, a tripropylsilyl group, a propyldimethylsilyl group, a tributylsilyl group, a t-butyldimethylsilyl group, a tripentylsilyl group, a triheptylsilyl group, and a trihexylsilyl group. Of those, a trimethylsilyl group and a triethylsilyl group are preferred. The alkyl groups substituting the silyl group may be identical to or different from each other.

In the general formulae (1) to (7), specific examples of the triarylsilyl group represented by each of R₁ to R₁₀ include a triphenylsilyl group, a trinaphthylsilyl group, and a trianthrylsilyl group. Of those, a triphenylsilyl group is preferred. The aryl groups substituting the silyl group may be identical to or different from each other.

In the general formulae (1) to (7), specific examples of the alkylarylsilyl group represented by each of R₁ to R₁₀ include a dimethylphenylsilyl group, a diethylphenylsilyl group, a dipropylphenylsilyl group, a dibutylphenylsilyl group, a dipentylphenylsilyl group, a diheptylphenylsilyl group, a dihexylphenylsilyl group, a dimethylnaphthylsilyl group, a dipropylnaphthylsilyl group, a dibutylnaphthylsilyl group, a dipentylnaphthylsilyl group, a diheptylnaphthylsilyl group, a dihexylnaphthylsilyl group, a dimethylanthrylsilyl group, a diethylanthrylsilyl group, a dipropylanthrylsilyl group, a dibutylanthrylsilyl group, a dipentylanthrylsilyl group, a diheptylanthrylsilyl group, a dihexylanthrylsilyl group, and a diphenylmethyl group. Of those, a dimethylphenylsilyl group, a diethylphenylsilyl group, and a diphenylmethyl group are preferred.

In the general formulae (1) to (7), specific examples of the aryl group represented by each of R₁ to R₁₀ and Ar₁ to Ar₉ include a phenyl group, a 2-methylphenyl group, a 3-methylphenyl group, a 4-methylphenyl group, a 4-ethylphenyl group, a biphenylyl group, a 4-methylbiphenylyl group, a 4-ethylbiphenylyl group, a 4-cyclohexylbiphenylyl group, an anthracenyl group, a naphthacenyl group, a terphenyl group, a triphenylyl group, a 3,5-dichlorophenylyl group, a naphthyl group, a 5-methylnaphthyl group, a phenanthryl group, a chrysenyl group, a benzophenanthryl group, a terphenyl group, a benzanthranyl group, a benzochrysenyl group, a pentacenyl group, a picenyl group, a pentaphenyl group, a pyrenyl group, a chrysenyl group, a fluorenyl group, a 9,9-dimethylfluorenyl group, an indenyl group, an acenaphthylenyl group, a fluoranthenyl group, and a perylenyl group. Of those, a phenyl group, a biphenylyl group, and a naphthyl group are preferred.

In the general formulae (1) to (7), specific examples of the halogen atom represented by each of R₁ to R₁₀ include fluorine, chlorine, and bromine.

In the general formulae (1) to (7), specific examples of the arylene group having 6 to 50 ring carbon atoms represented by each of L₁ to L₅ include groups obtained by rendering the above-mentioned aryl groups divalent.

Examples of the substituent of each of the above-mentioned groups that may each have a substituent include a linear or branched alkyl group having 1 to 10 carbon atoms, a cycloalkyl group having 3 to 10 ring carbon atoms, a trialkylsilyl group having 3 to 10 carbon atoms, a triarylsilyl group having 18 to 30 ring carbon atoms, an alkylarylsilyl group having 8 to 15 carbon atoms (whose aryl moiety has 6 to 14 ring carbon atoms), an aryl group having 6 to 14 ring carbon atoms, and a halogen atom.

Specific examples of the linear or branched alkyl group having 1 to 10 carbon atoms, the cycloalkyl group having 3 to 10 ring carbon atoms, the trialkylsilyl group having 3 to 10 carbon atoms, the triarylsilyl group having 18 to 30 ring carbon atoms, the alkylarylsilyl group having 8 to 15 carbon atoms (whose aryl moiety has 6 to 14 ring carbon atoms), the aryl group having 6 to 14 ring carbon atoms, or the halogen atom as the substituent that may be possessed by each of the above-mentioned groups include the same examples as those given as specific examples of R₁ to R₁₀.

Shown below are specific examples of the compound represented by the general formula (1), but the compound is not limited thereto.

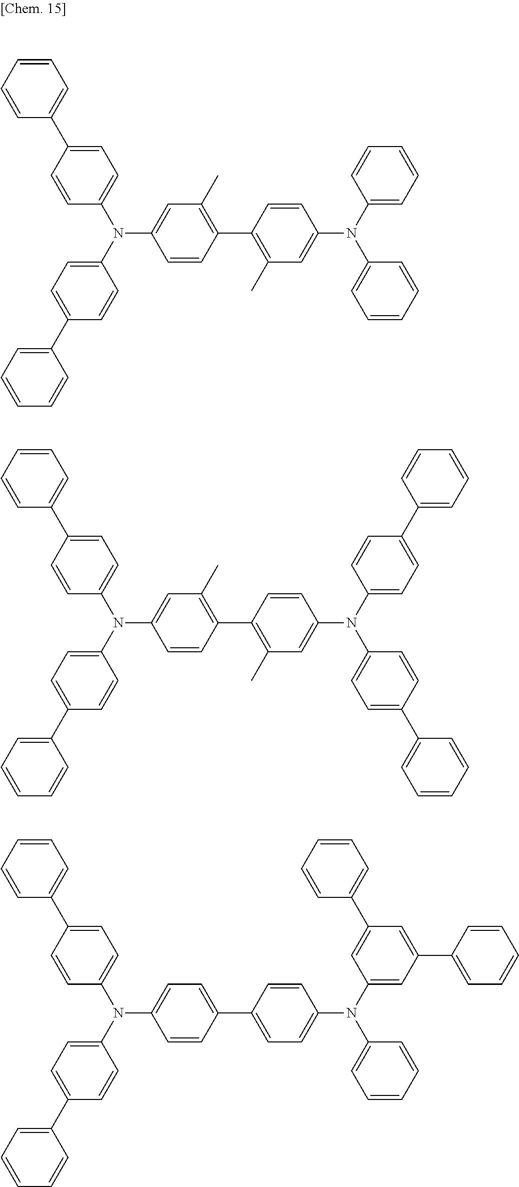

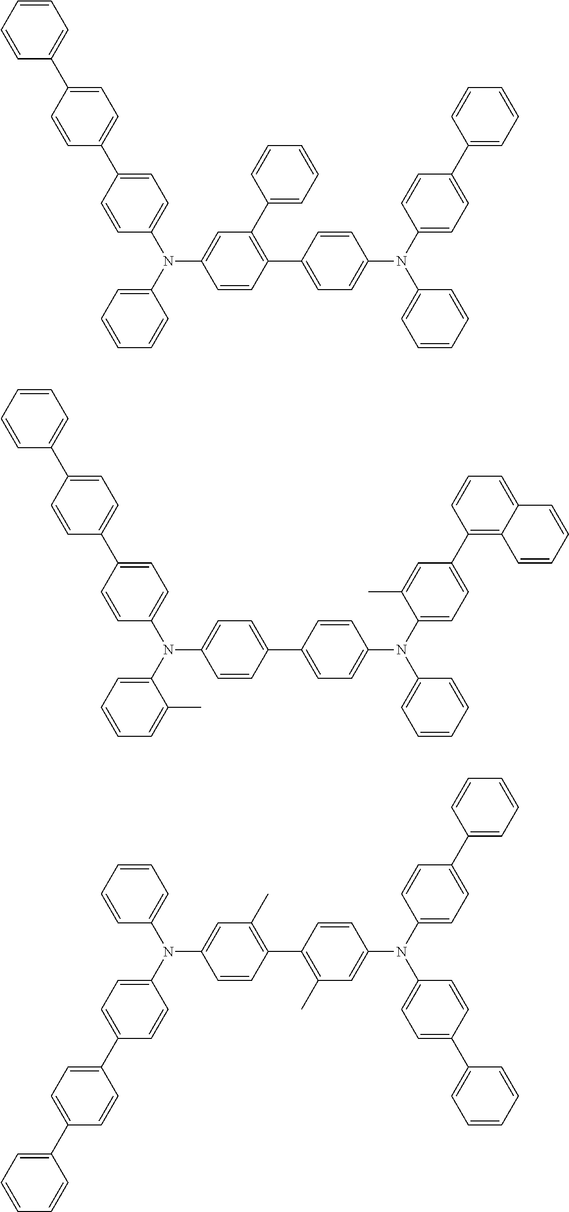

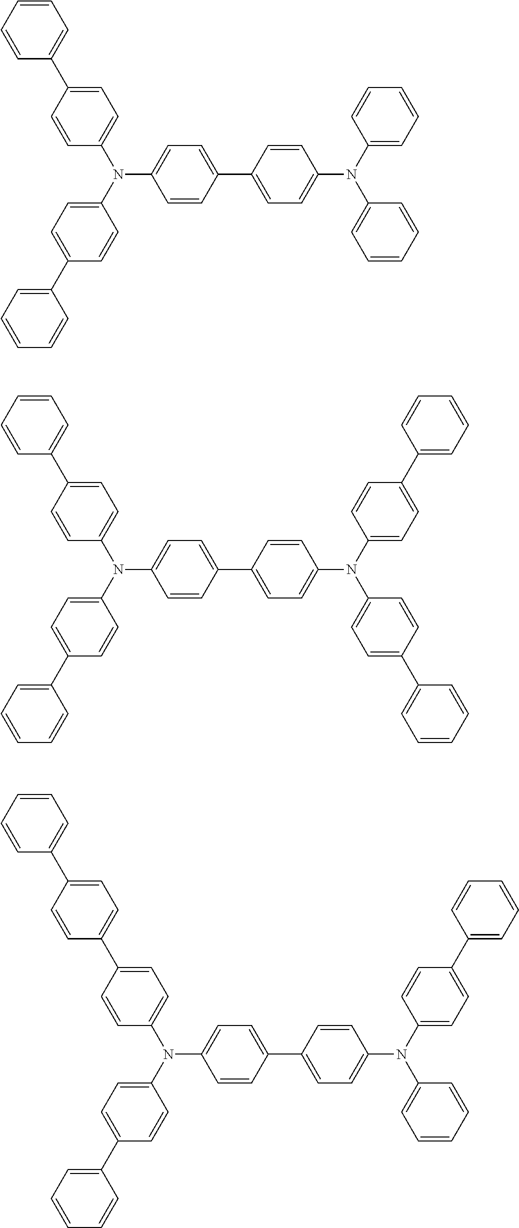

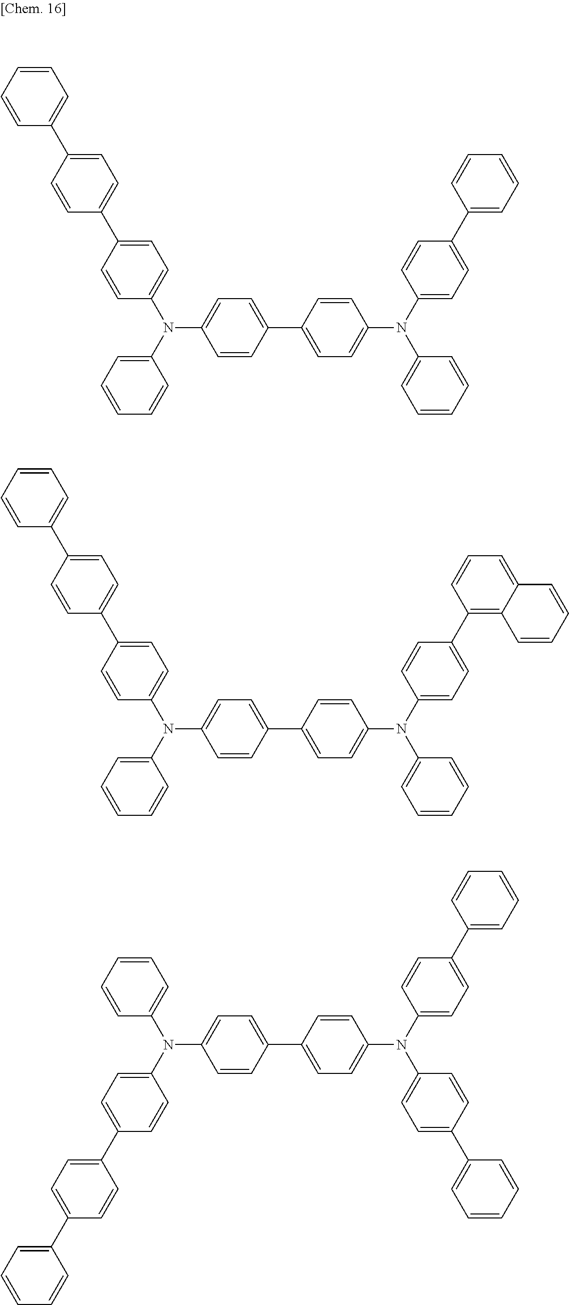

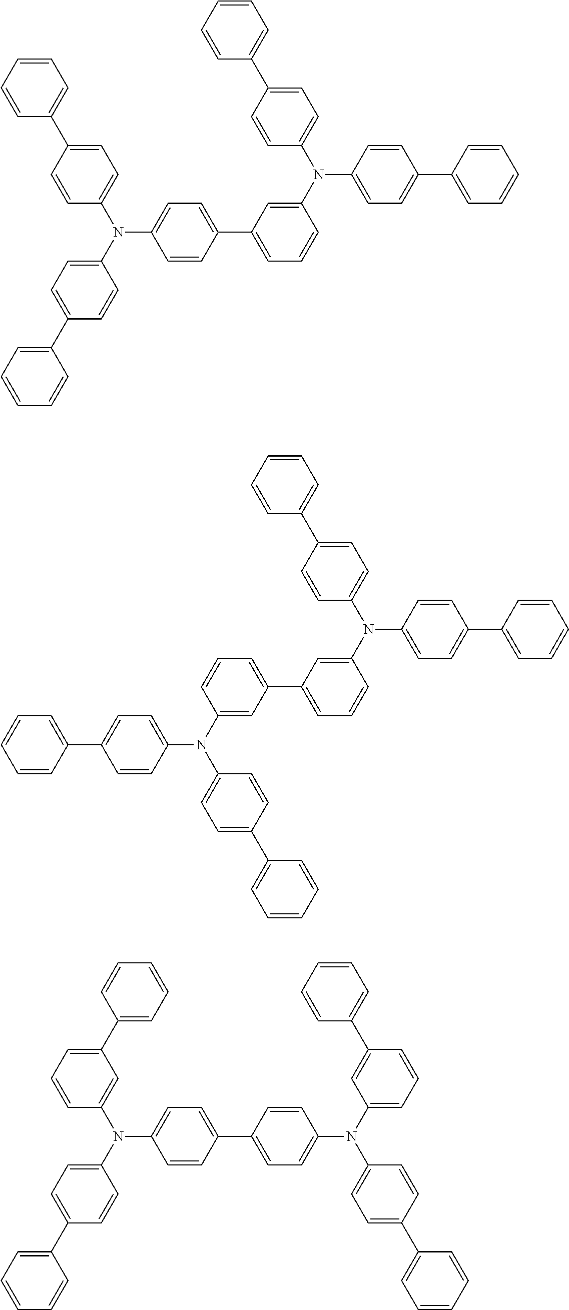

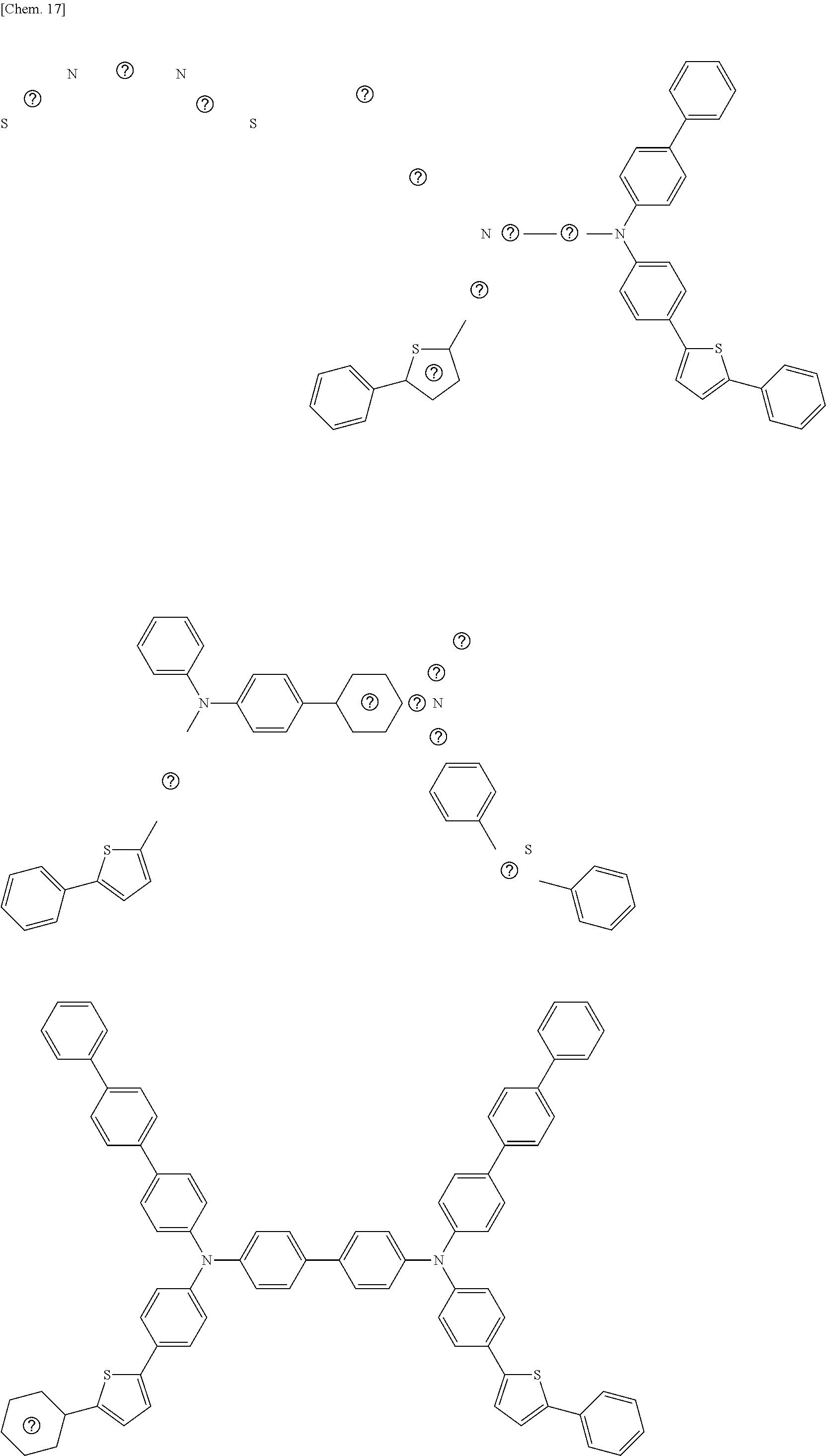

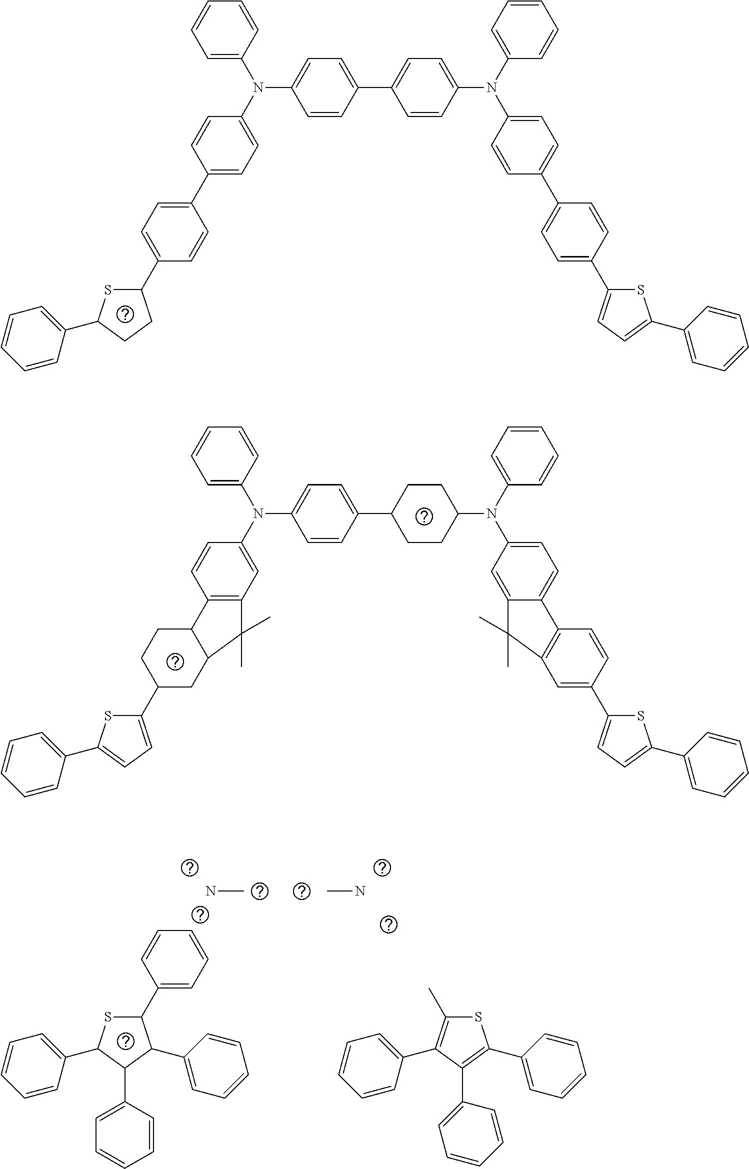

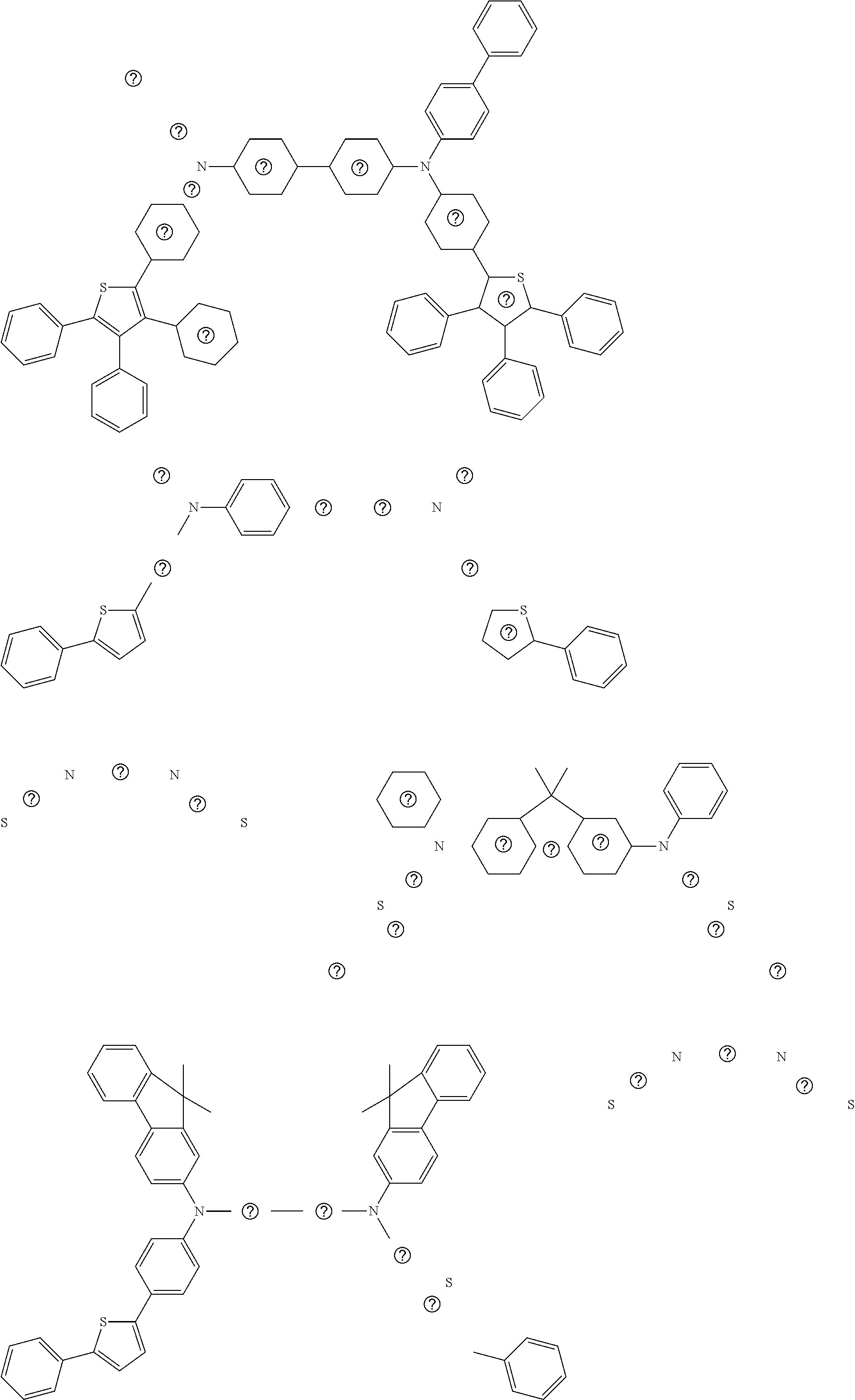

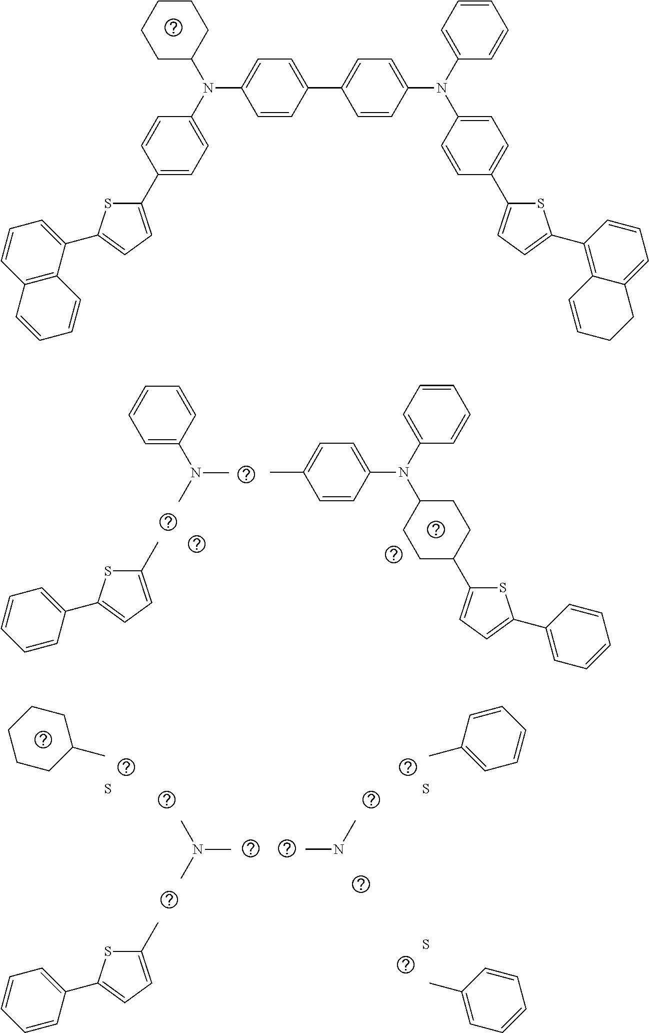

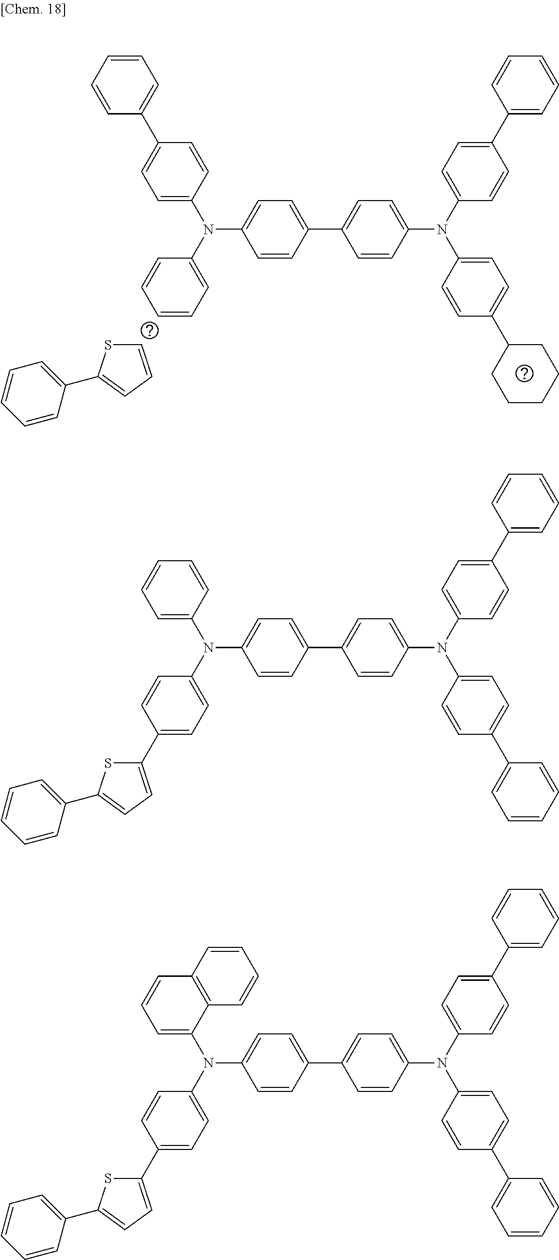

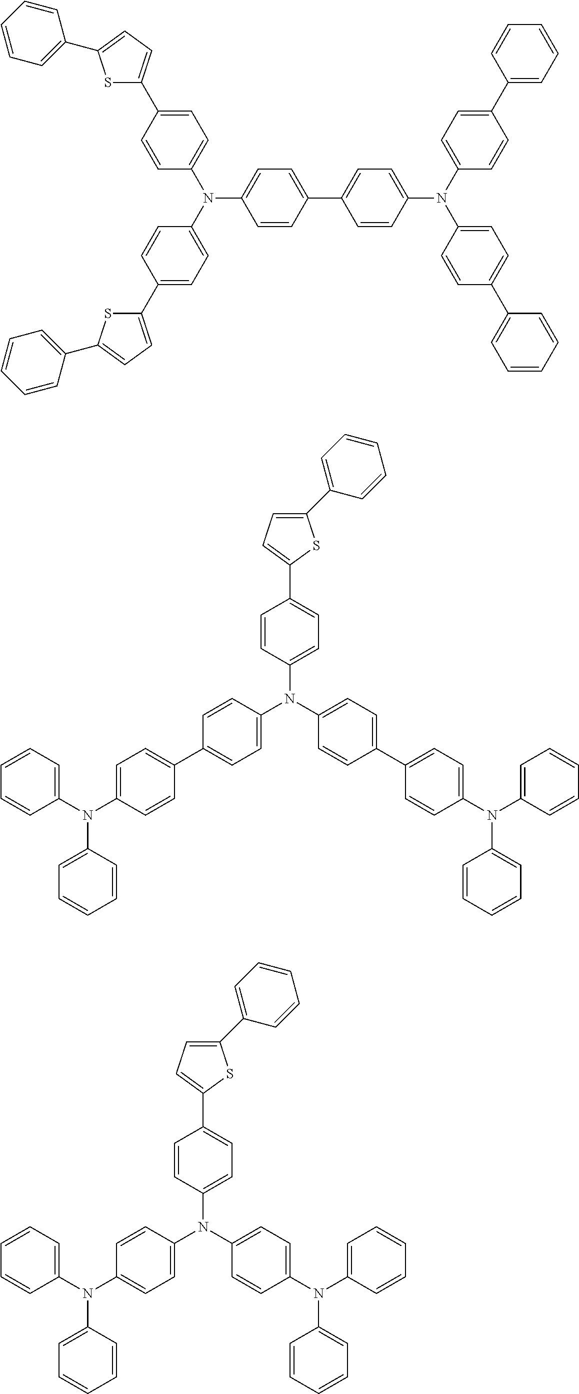

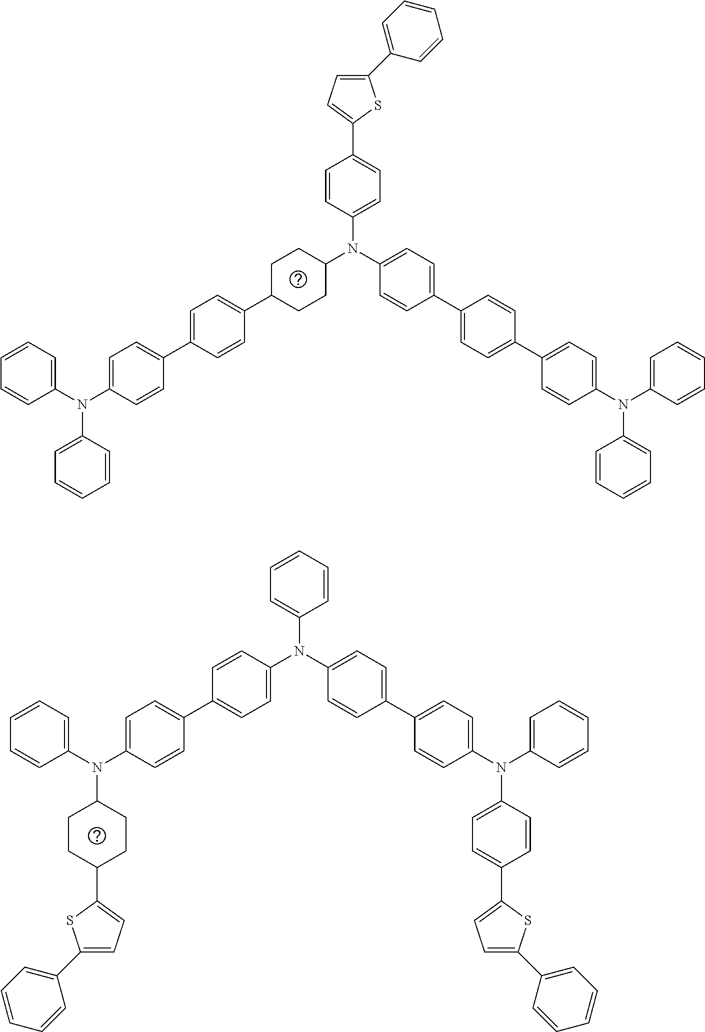

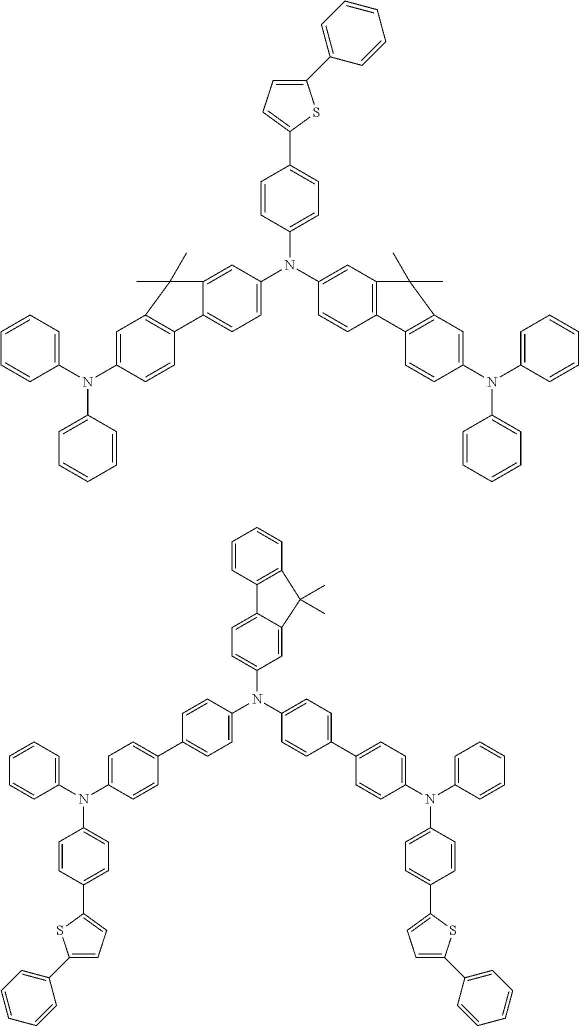

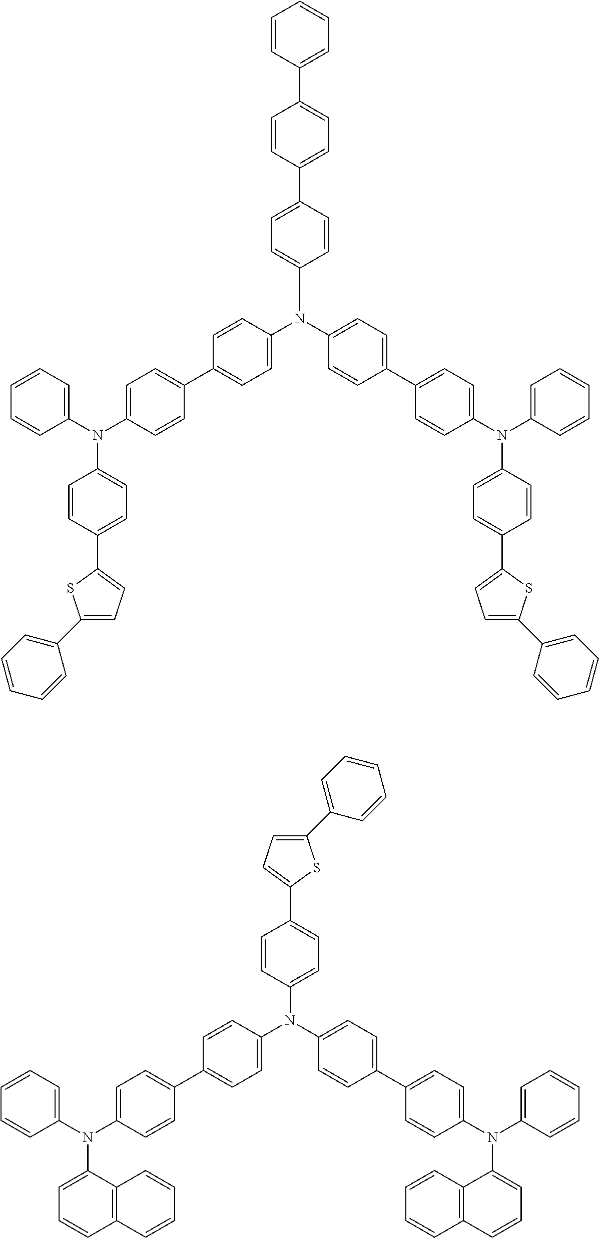

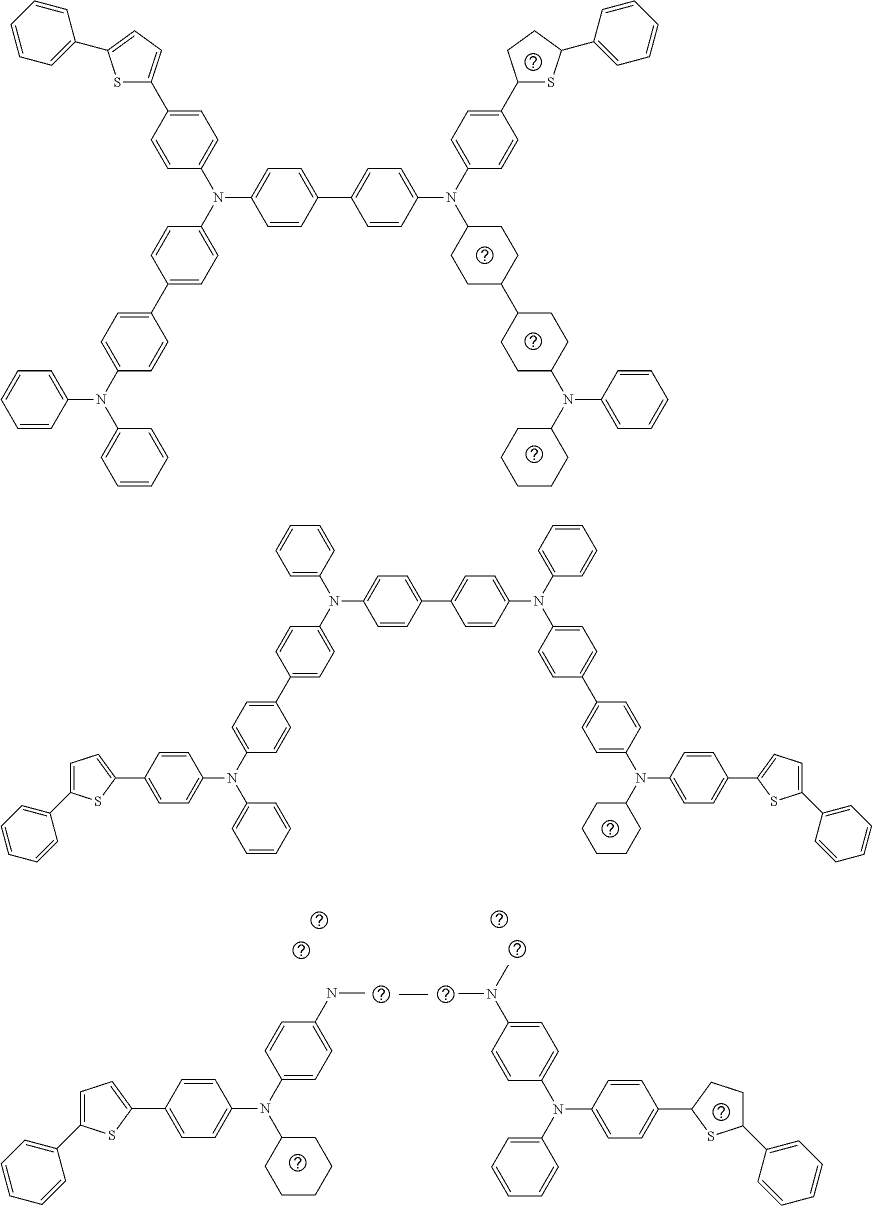

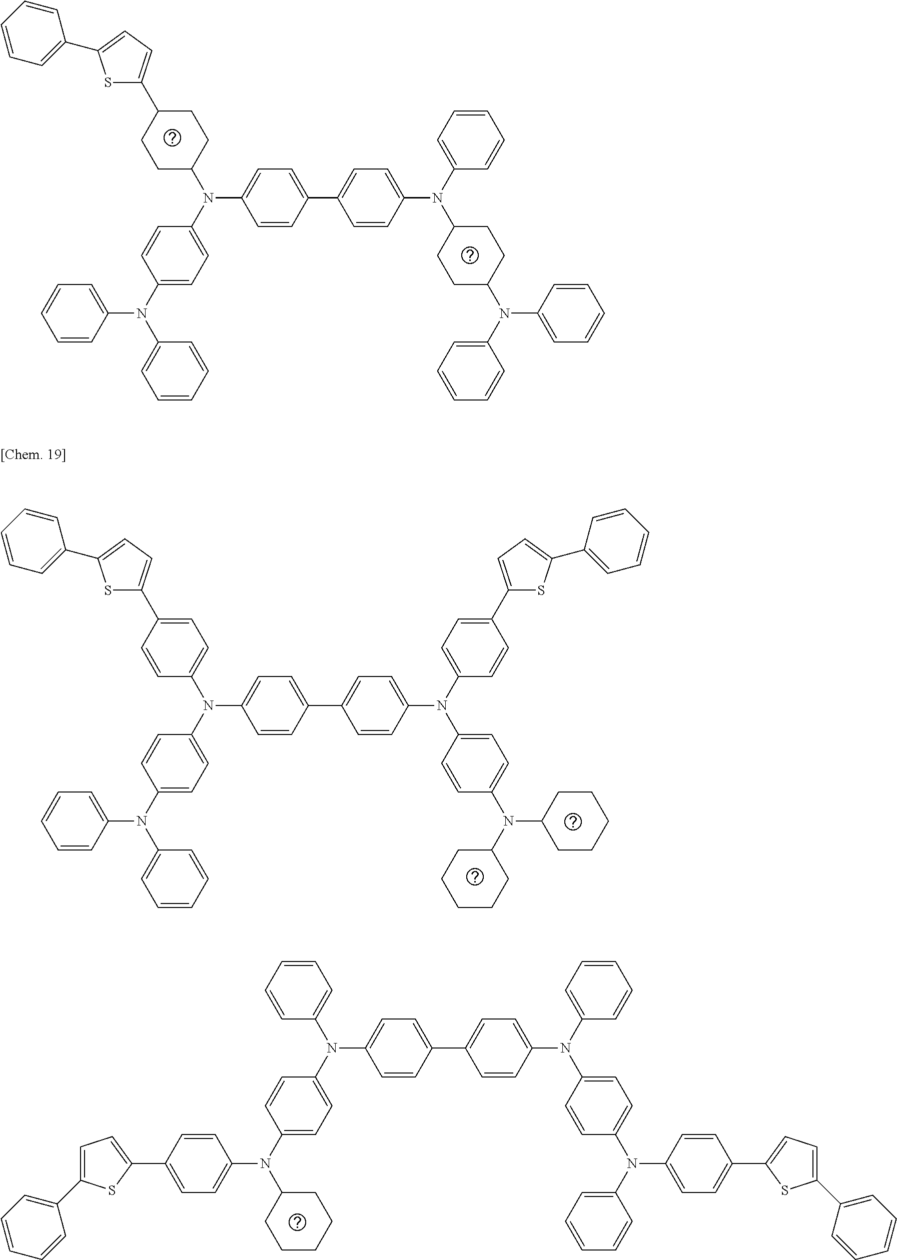

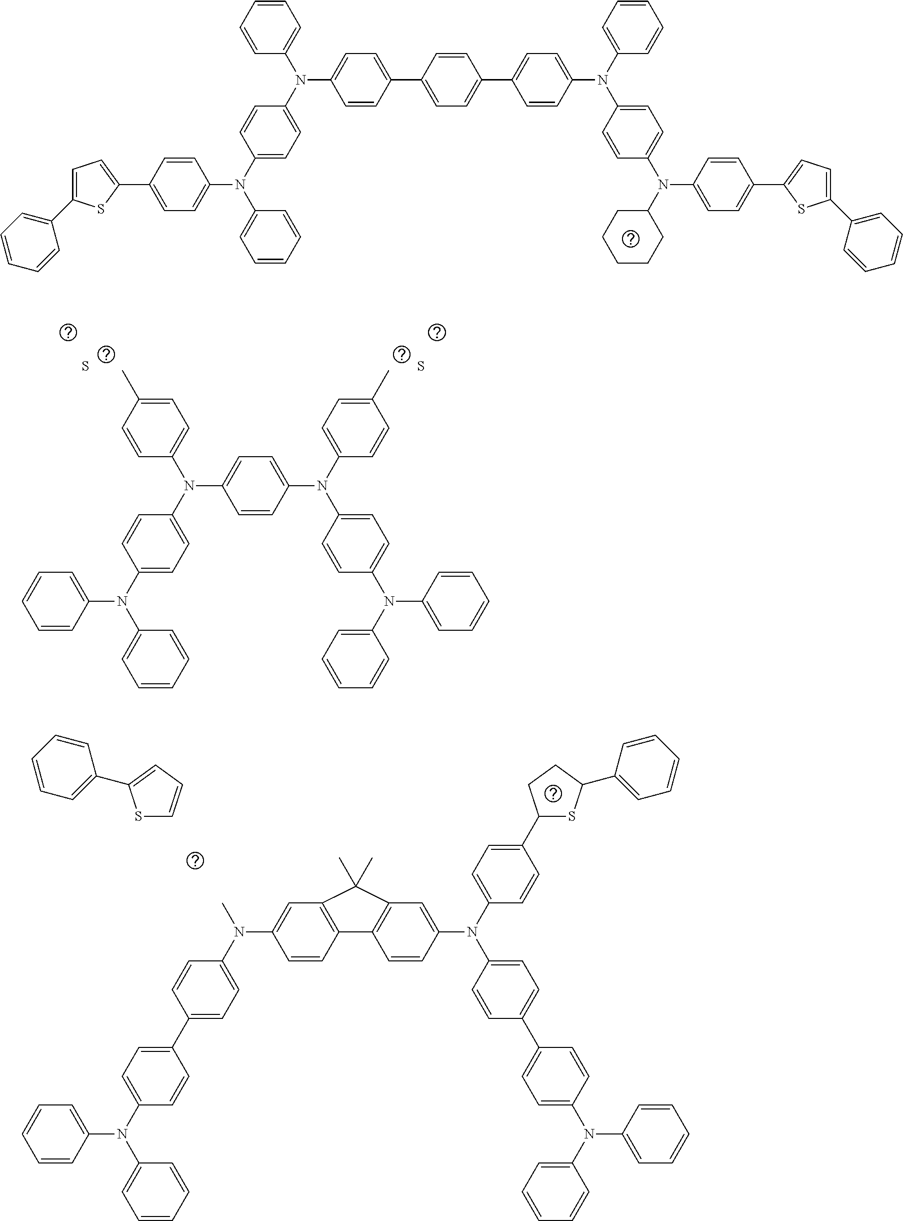

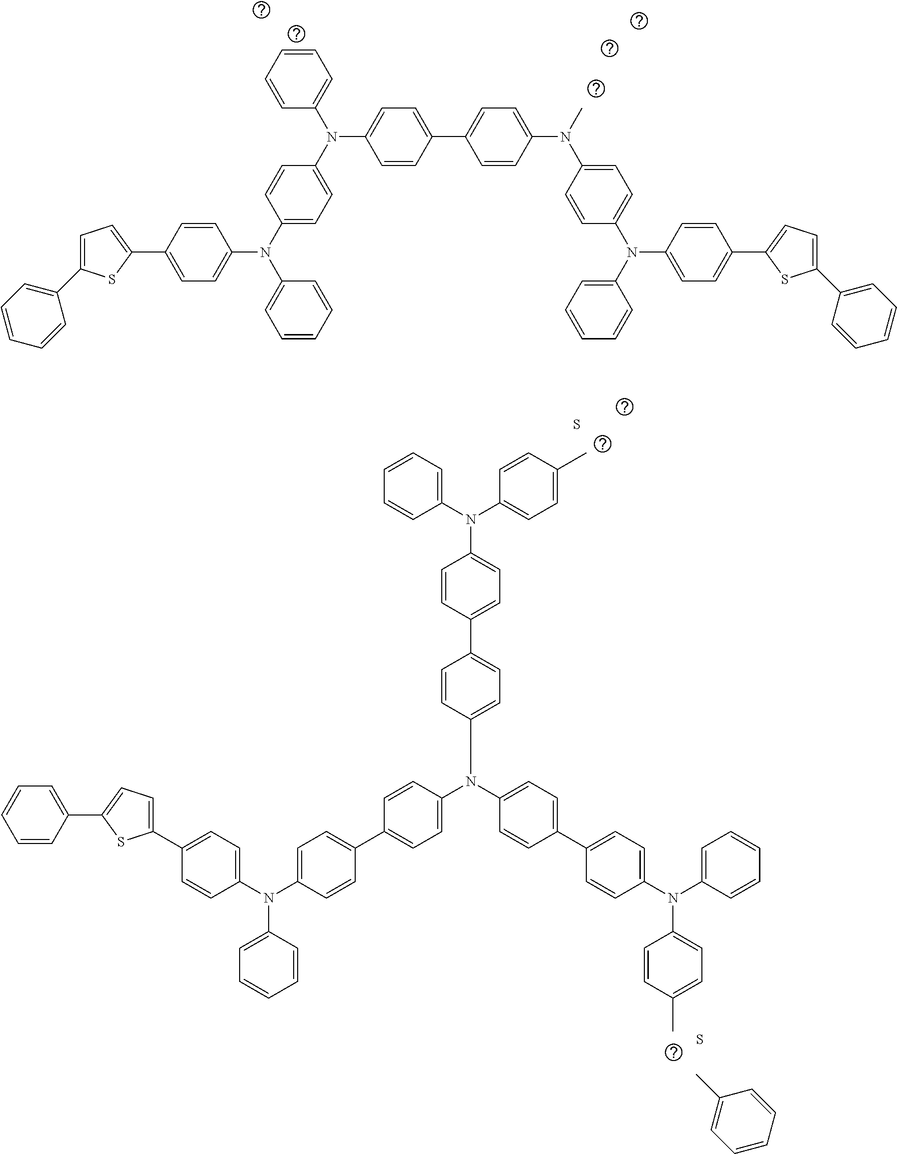

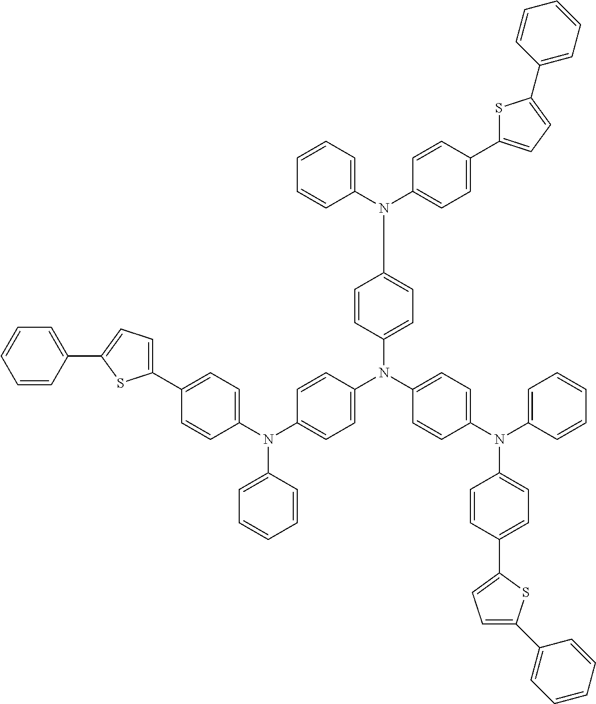

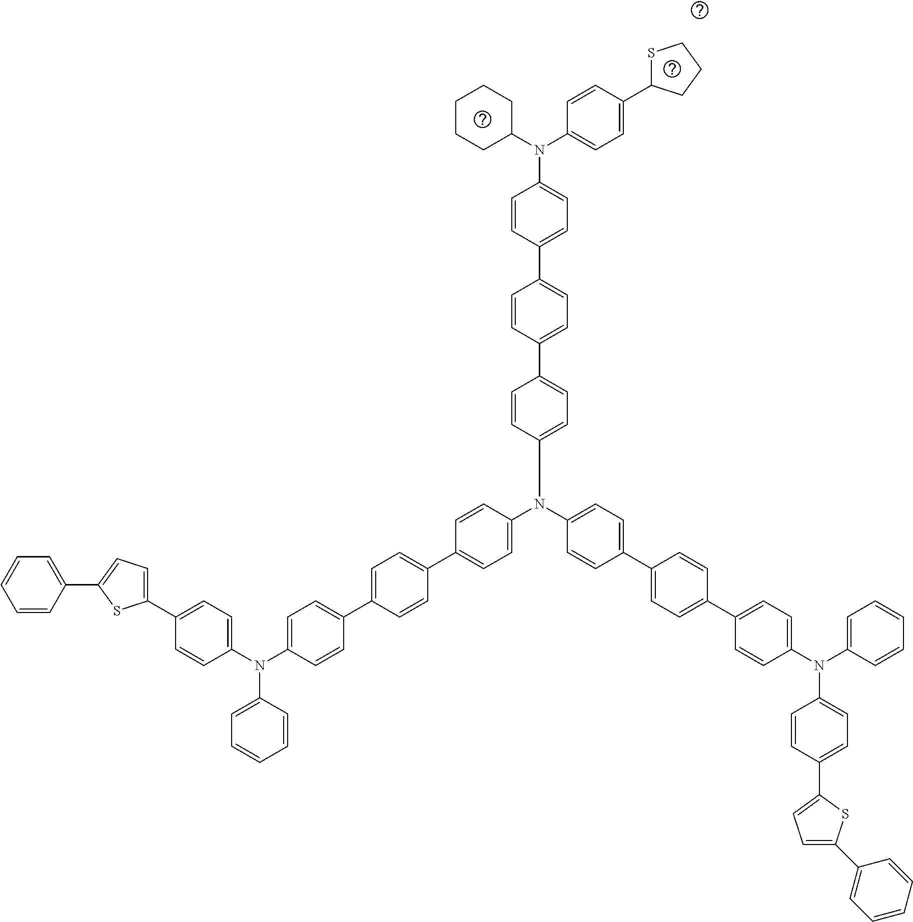

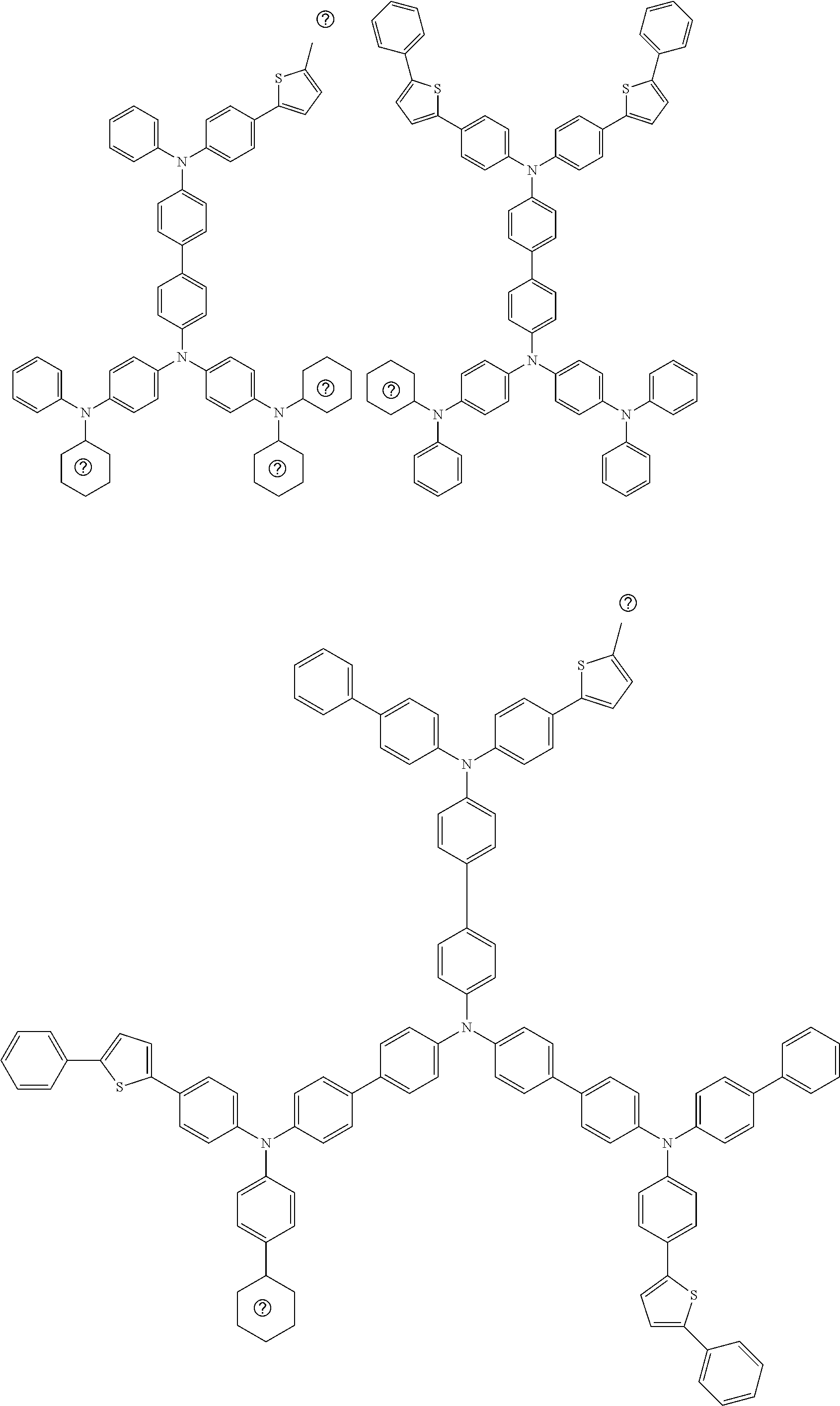

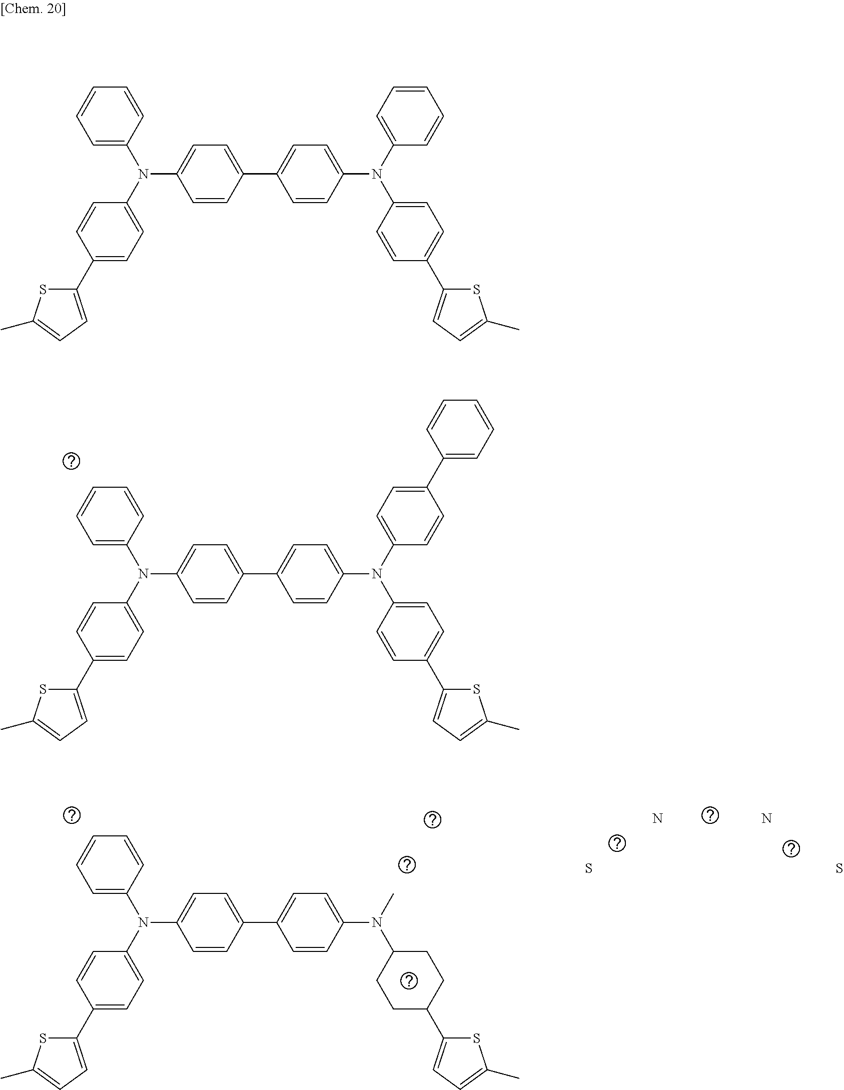

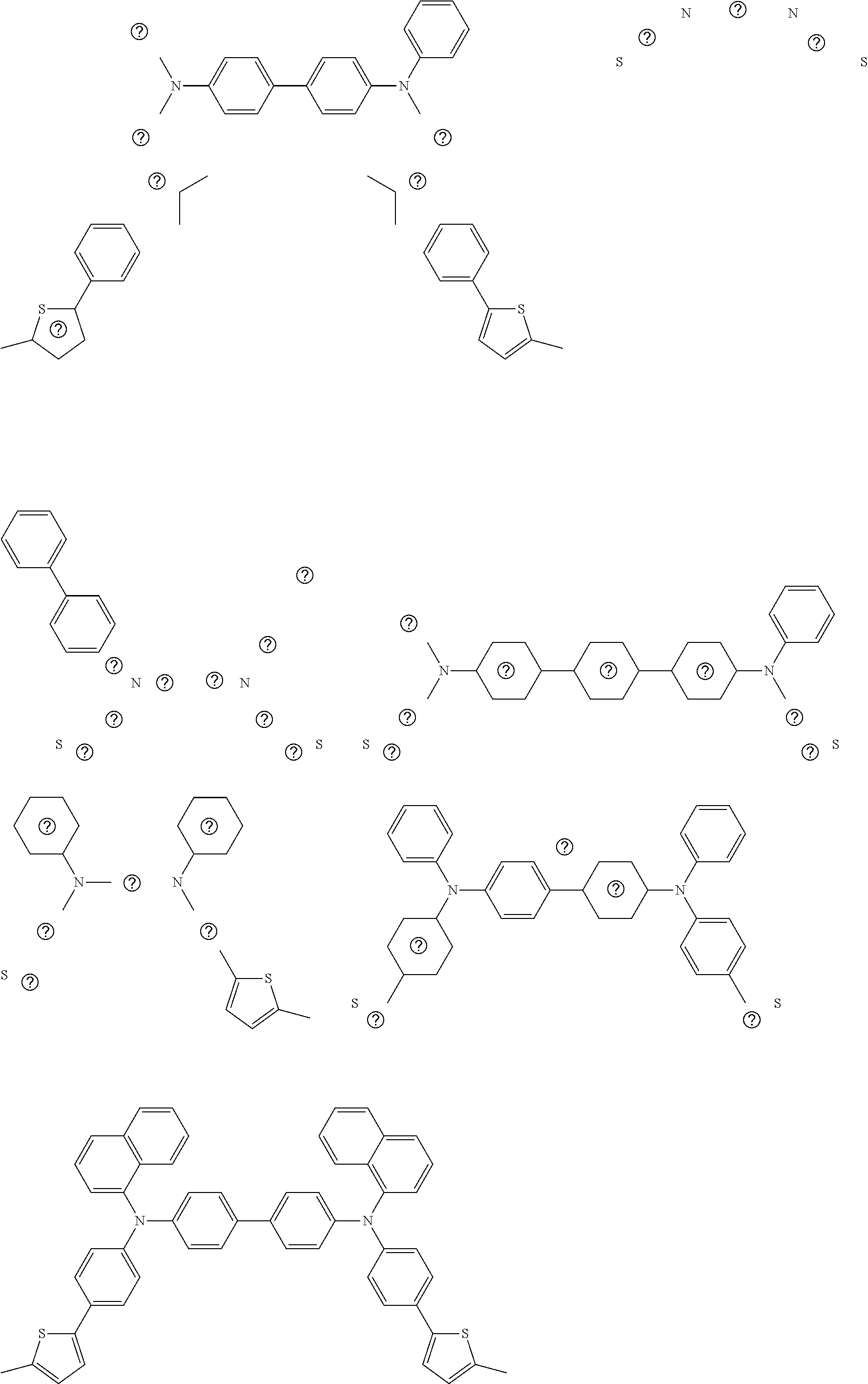

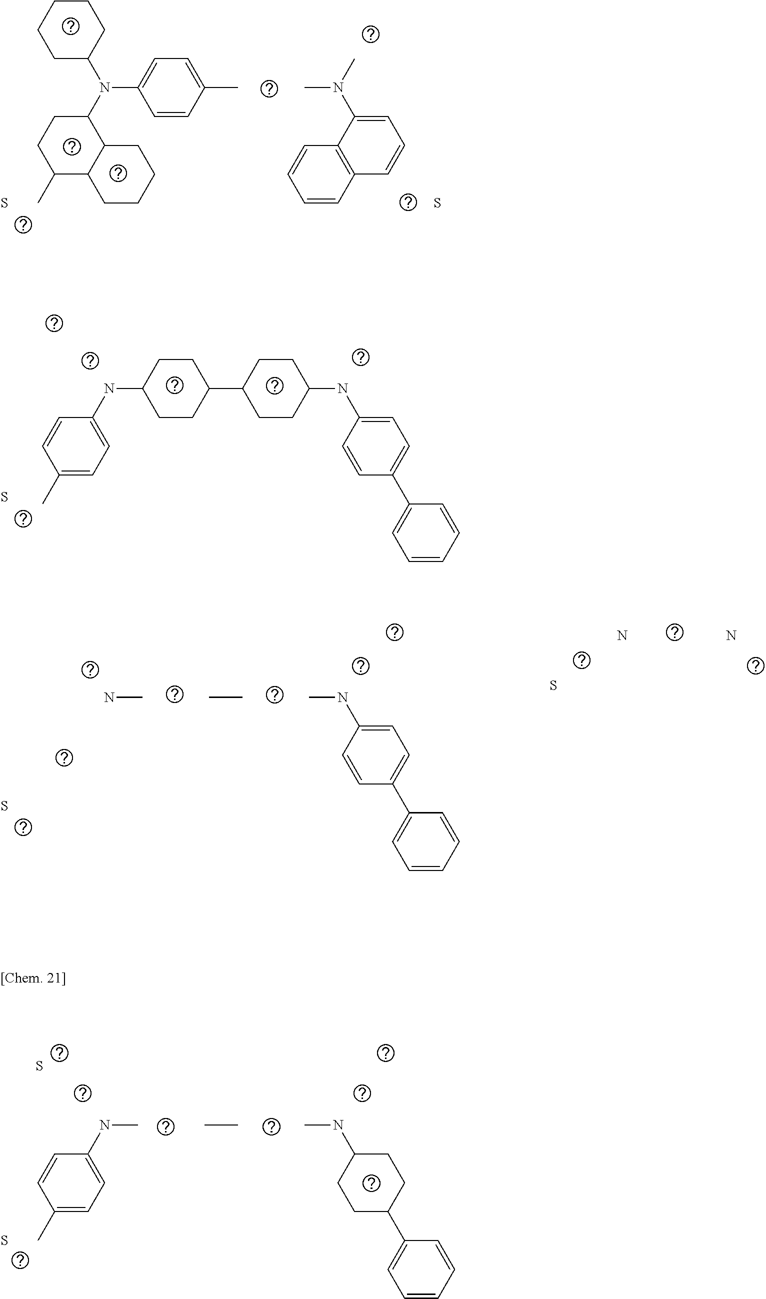

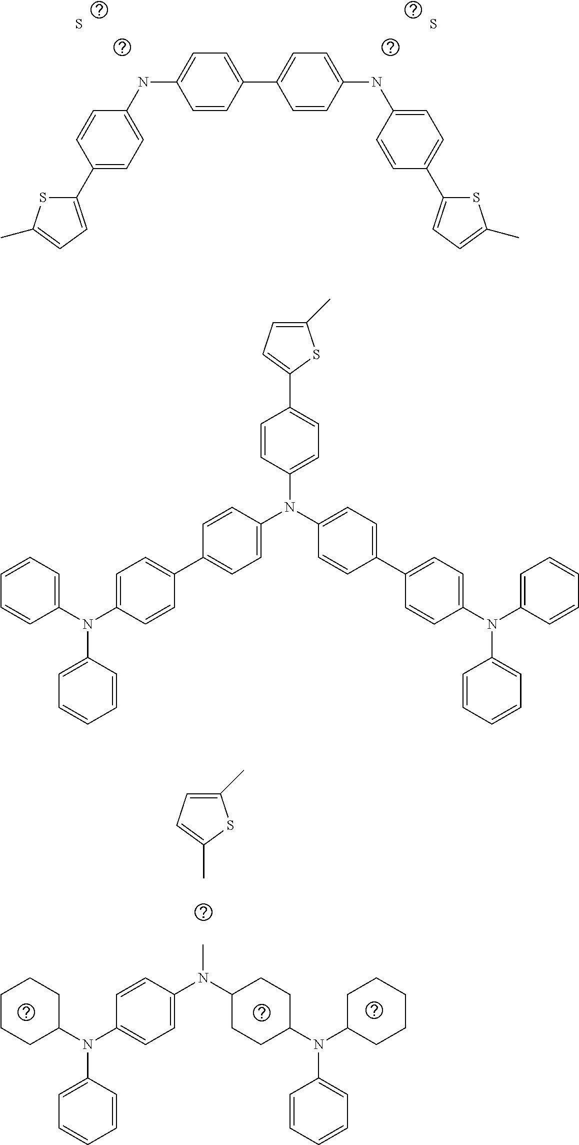

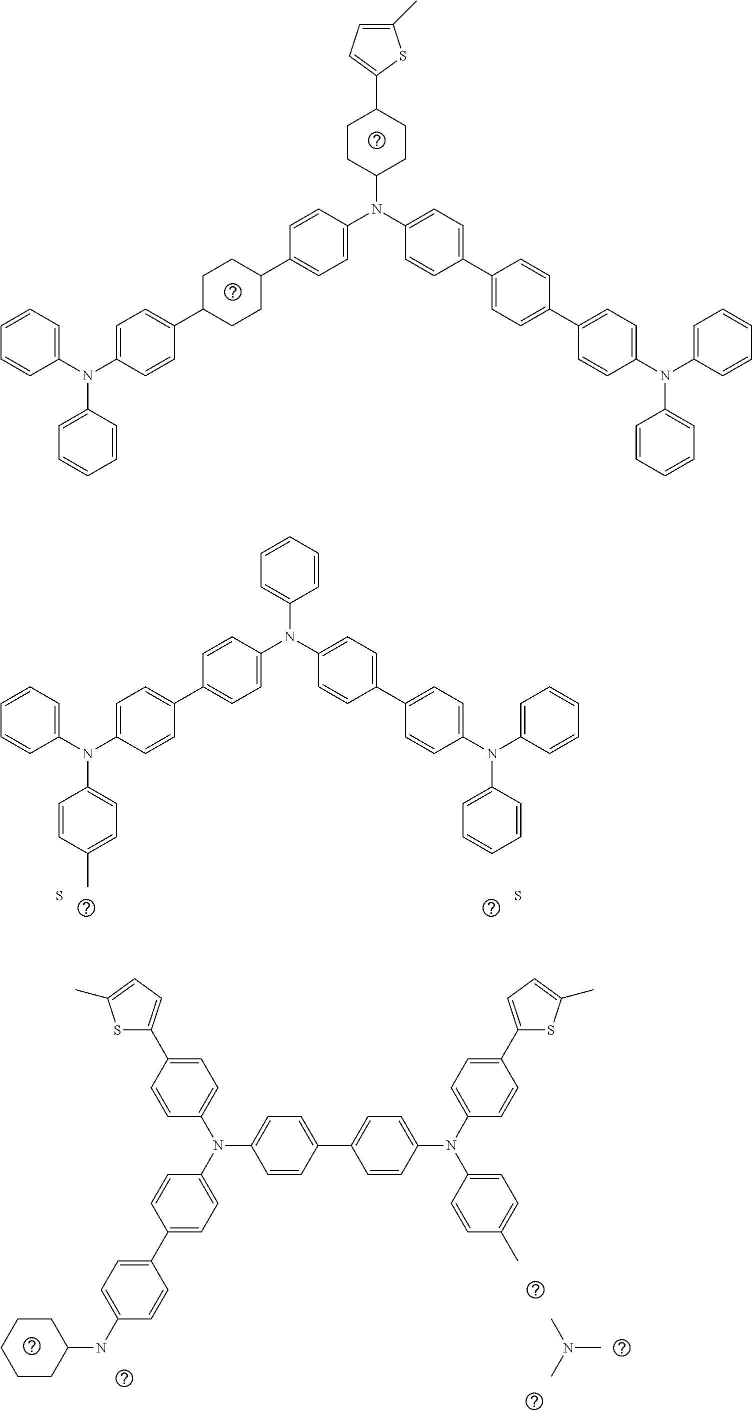

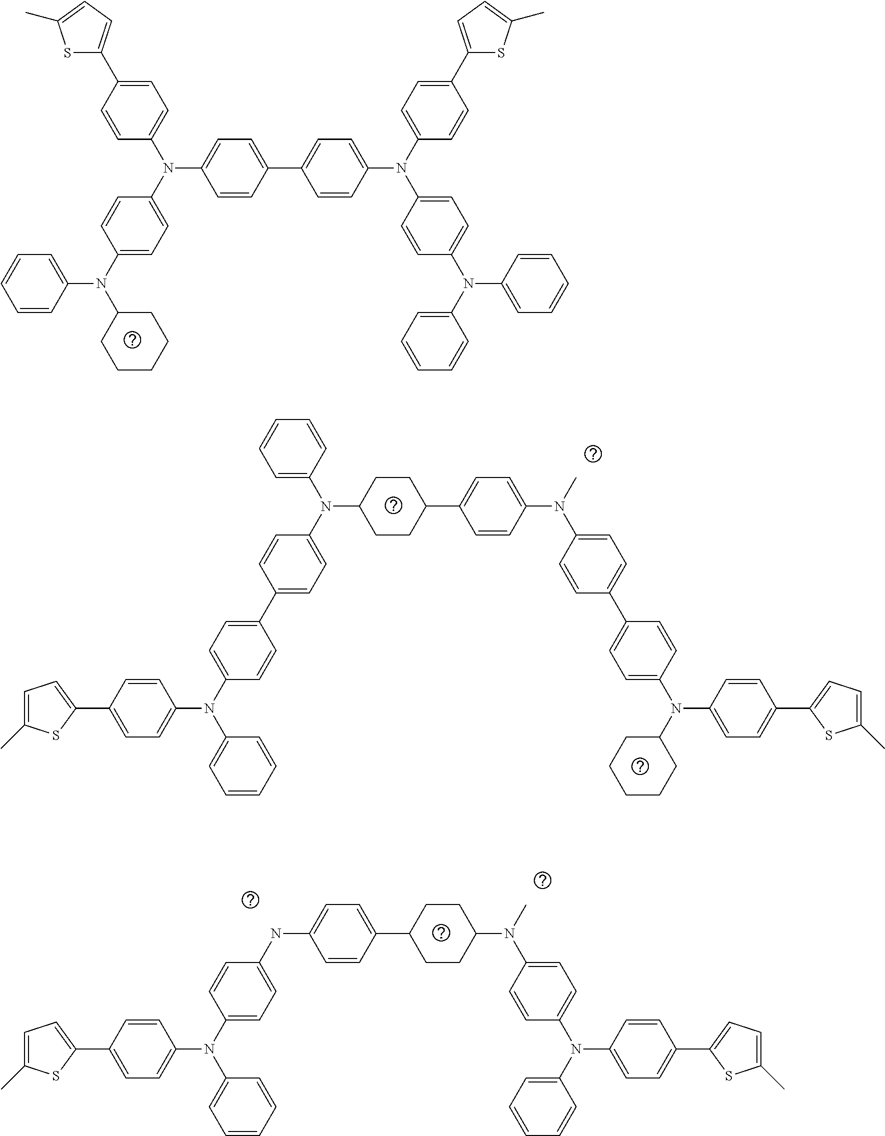

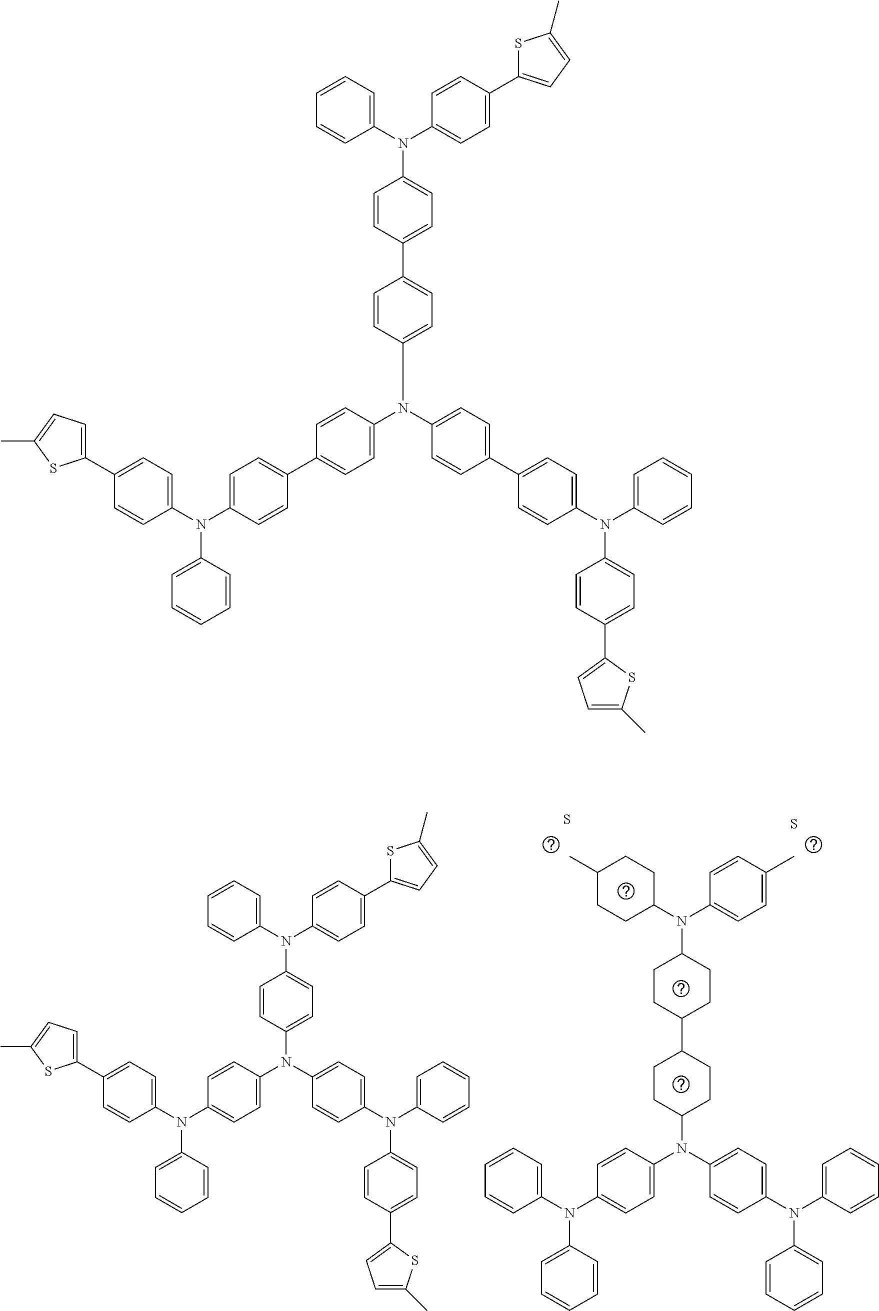

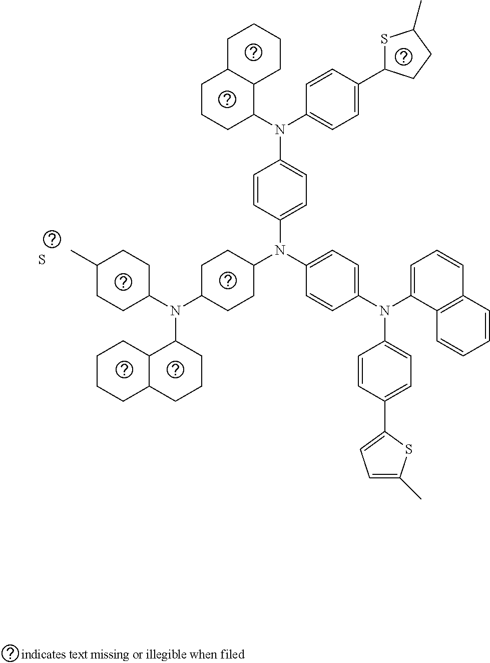

Shown below are specific examples of the compound represented by the general formula (2), but the compound is not limited thereto.

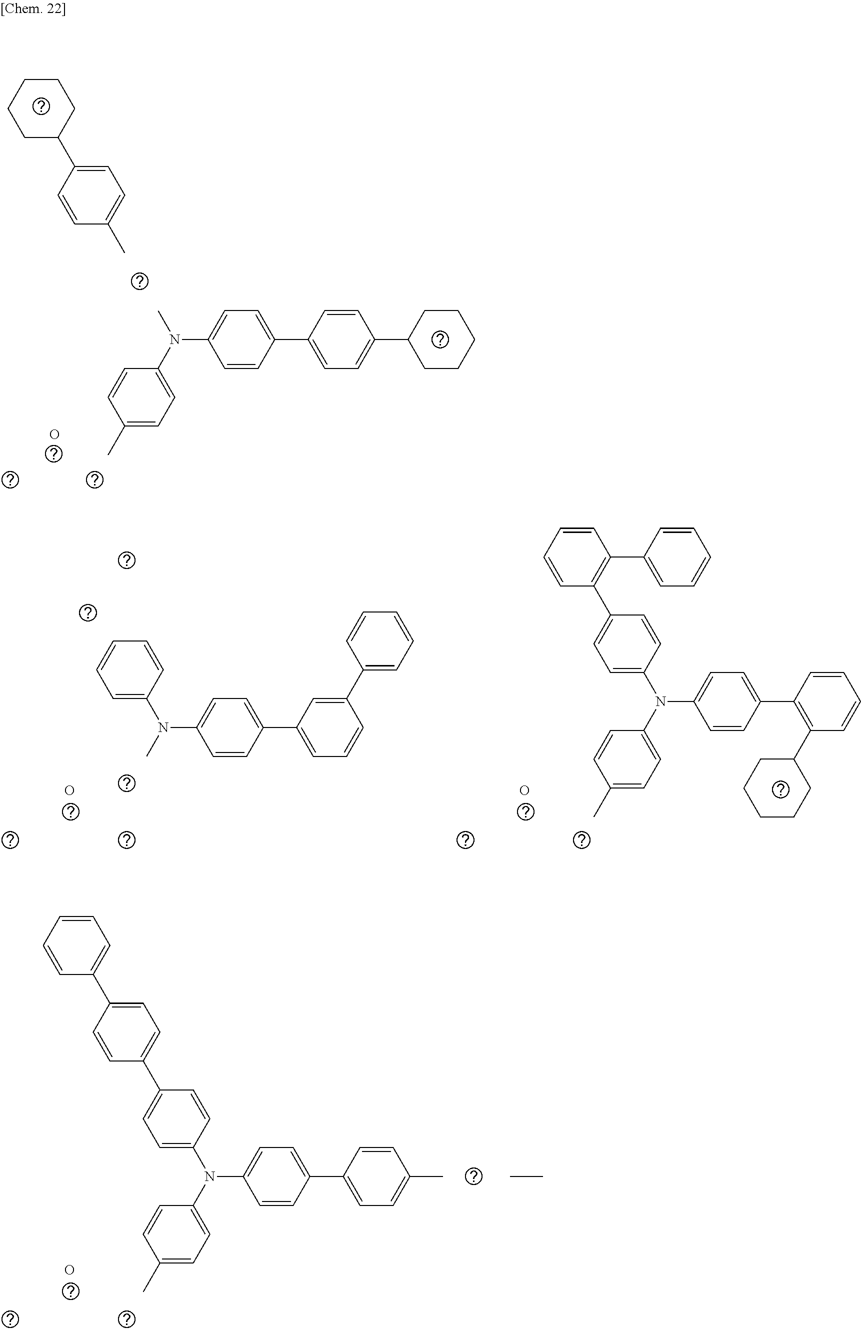

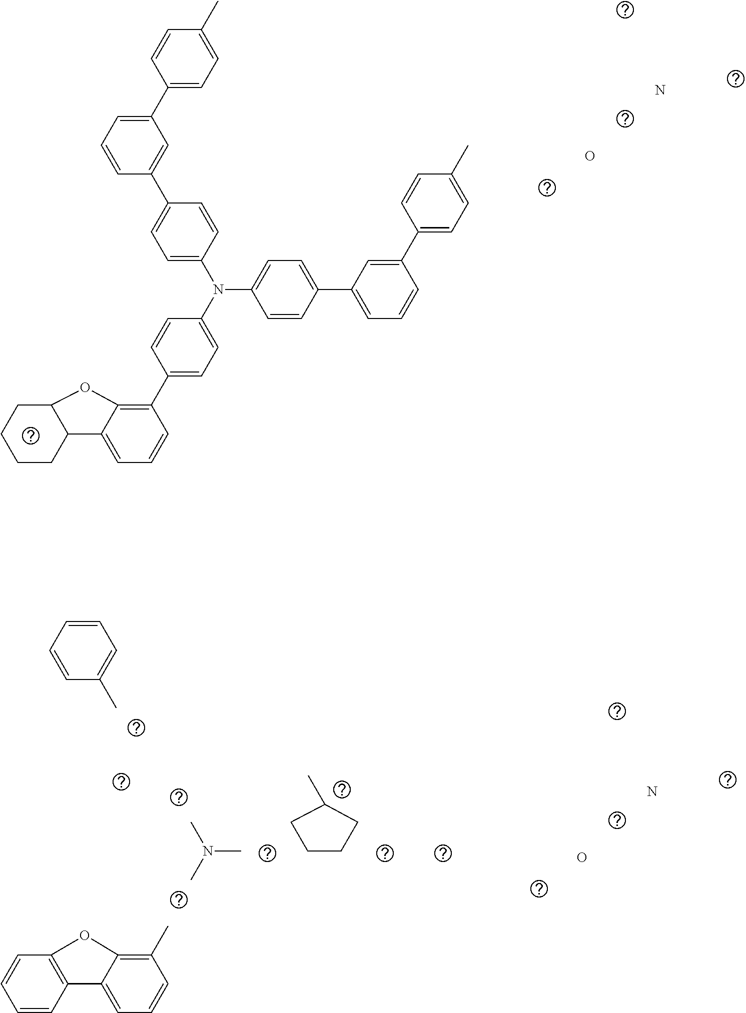

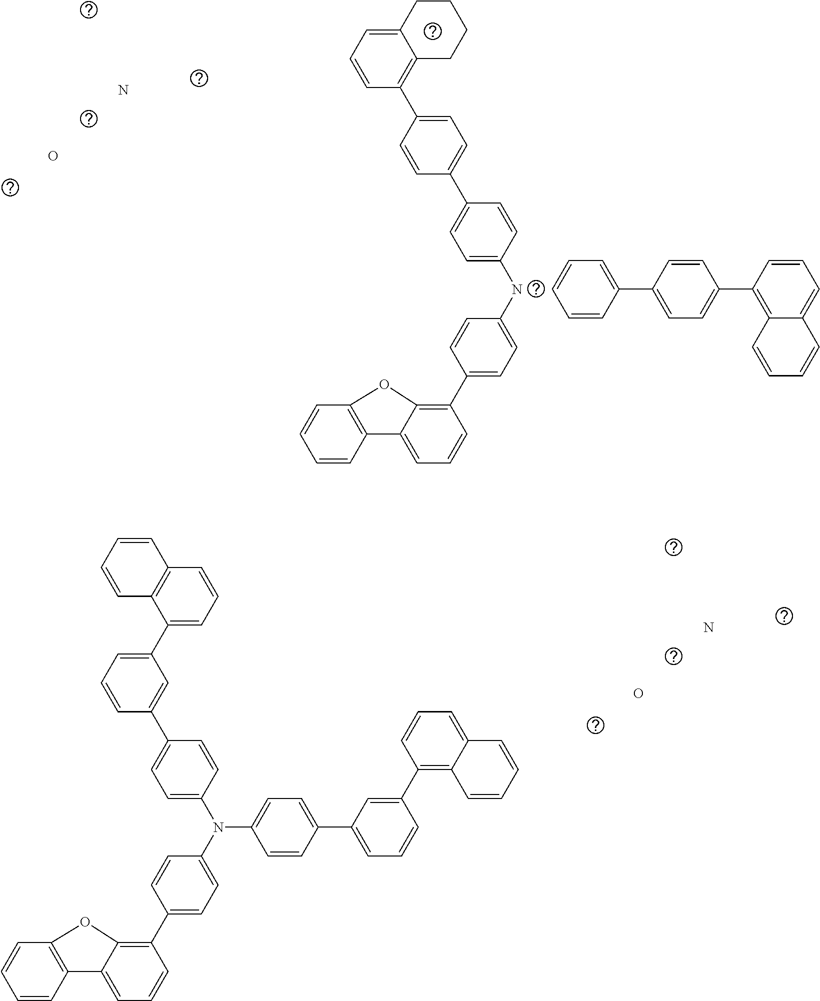

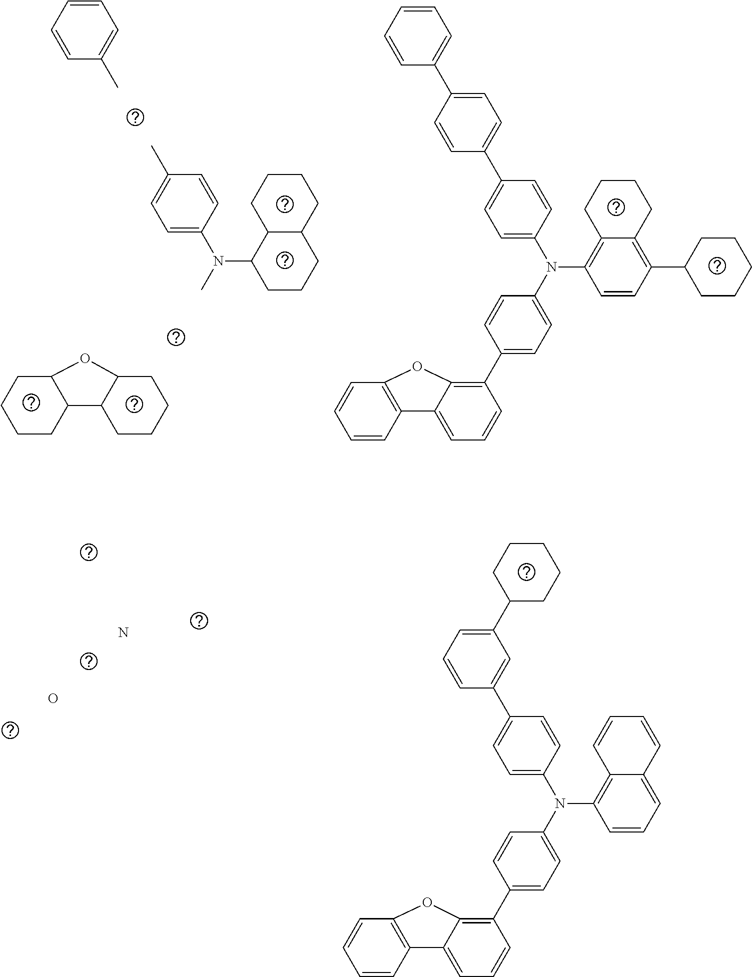

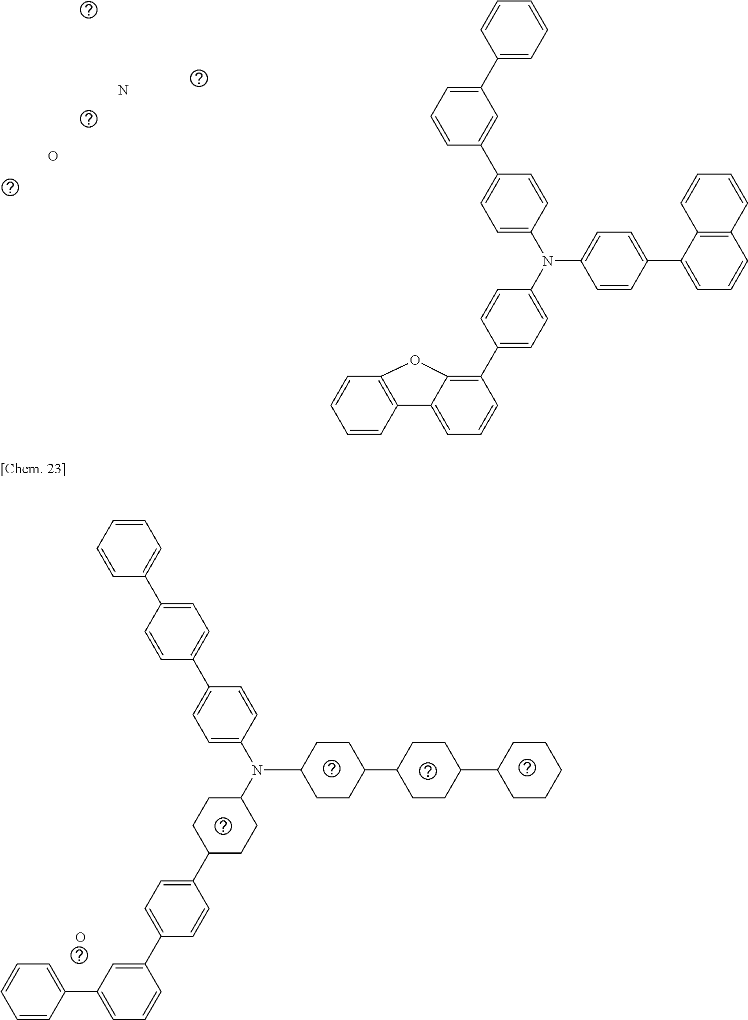

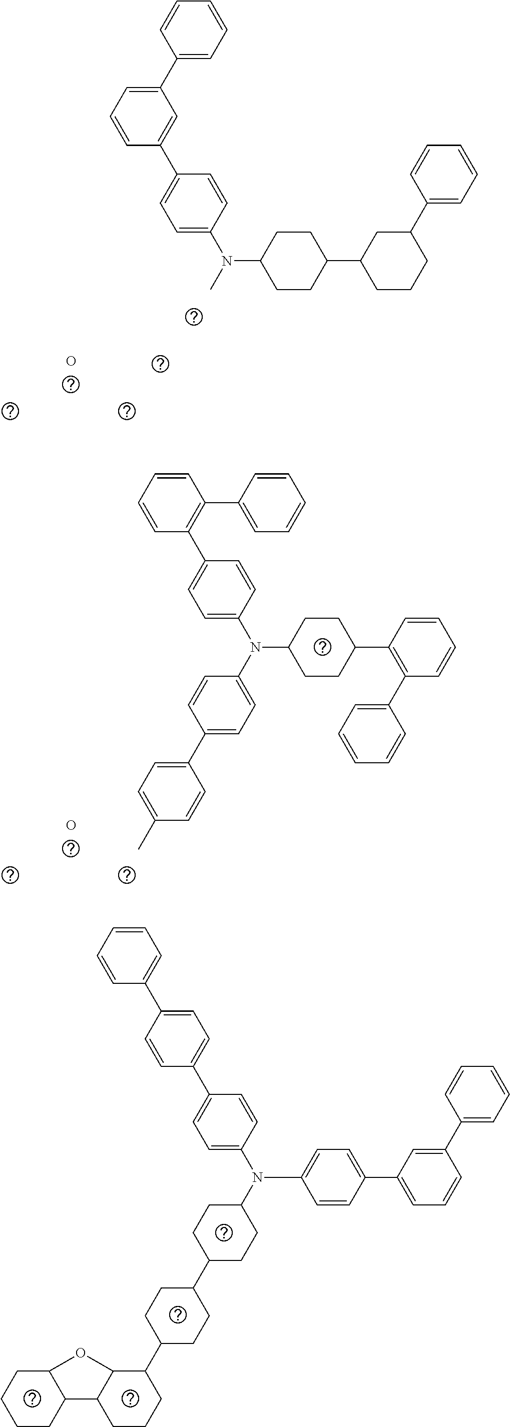

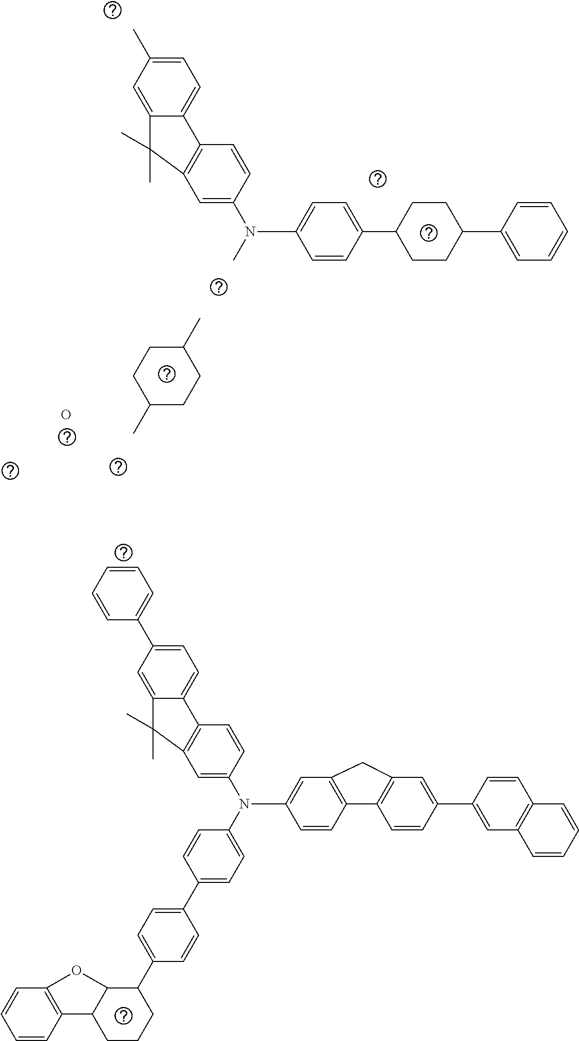

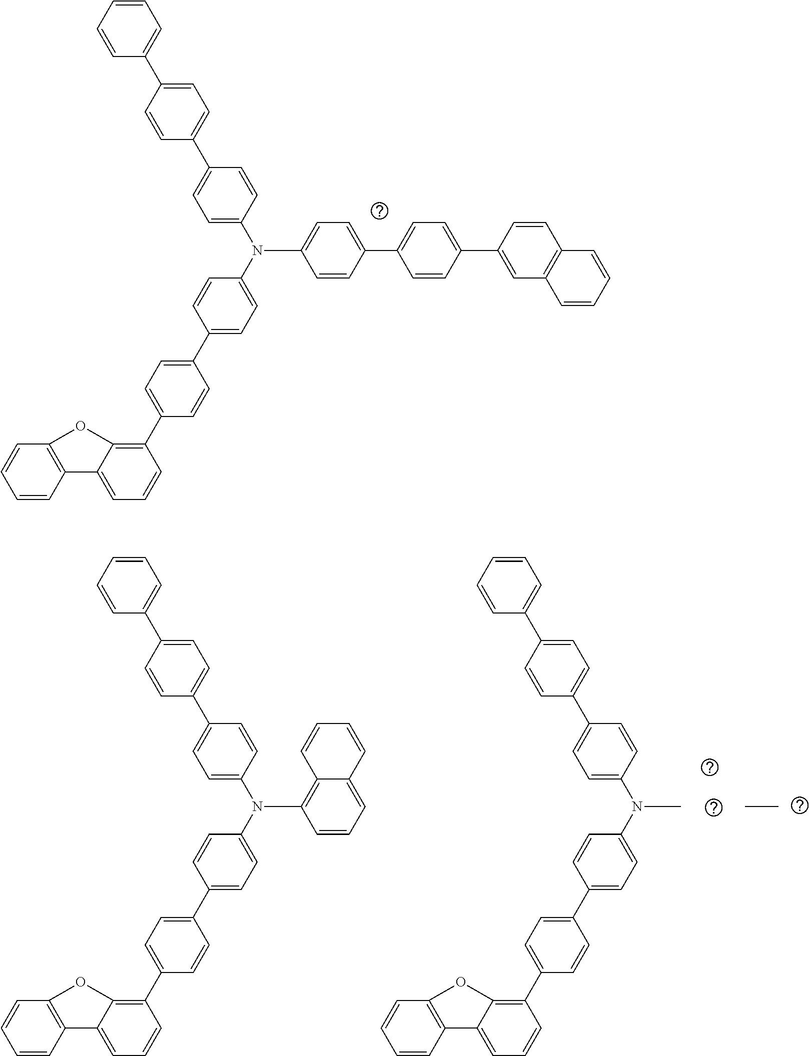

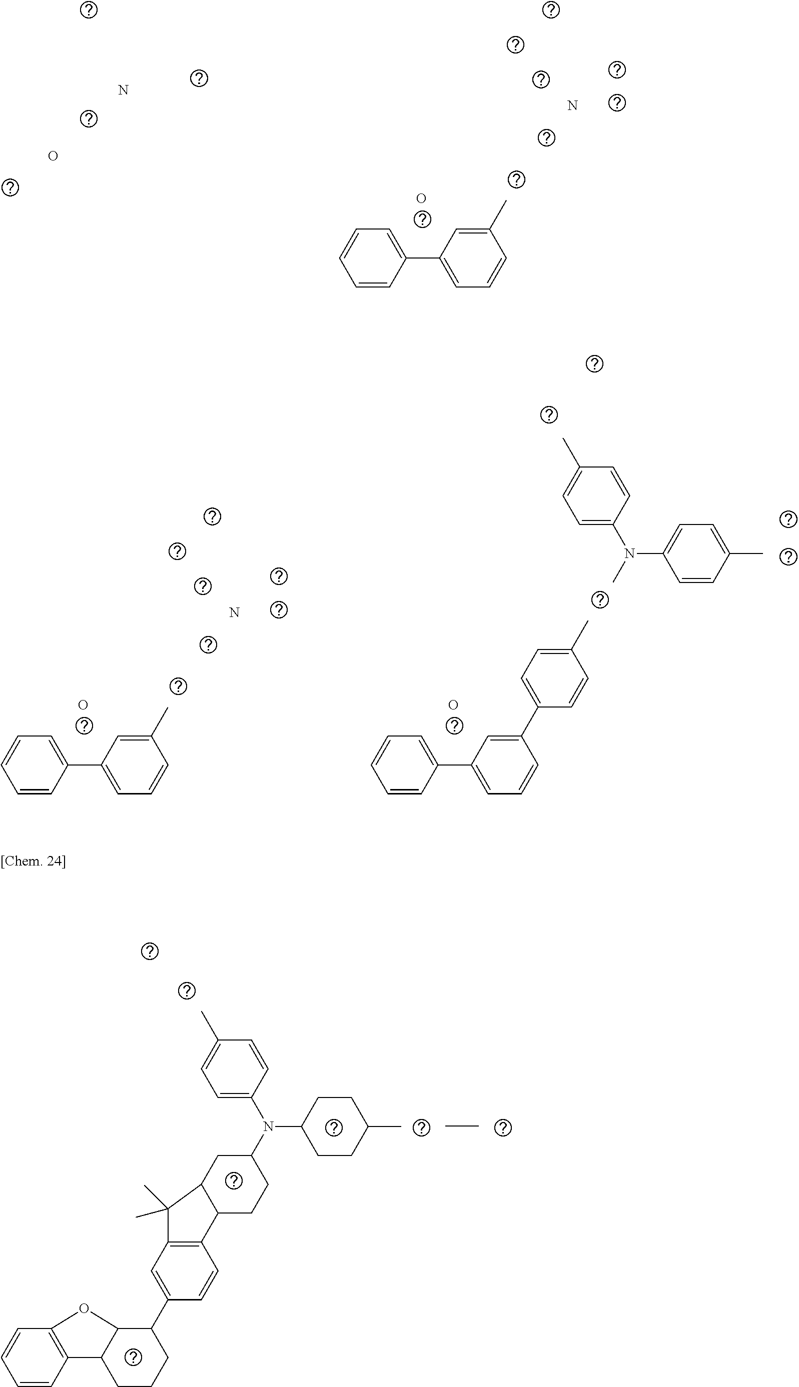

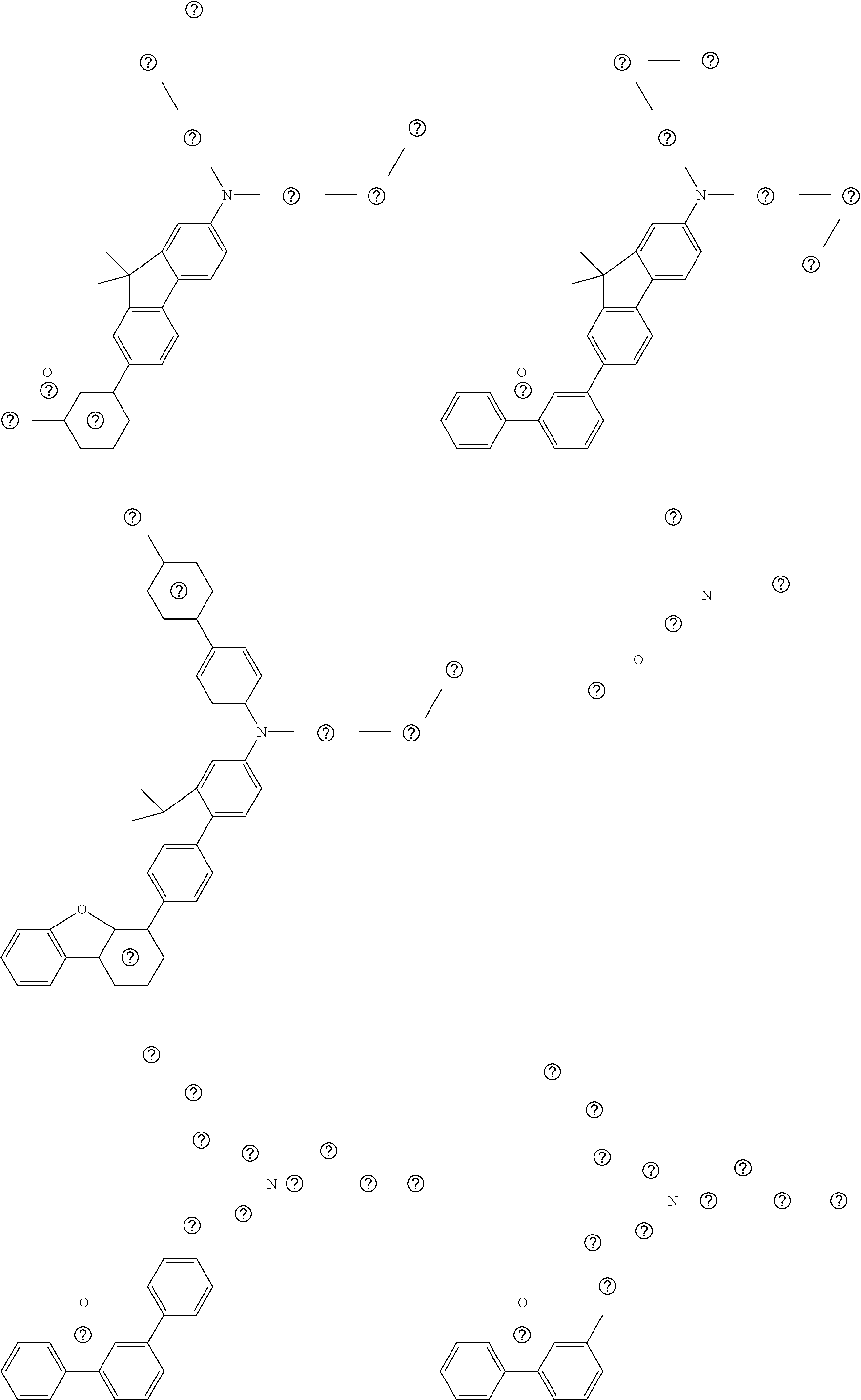

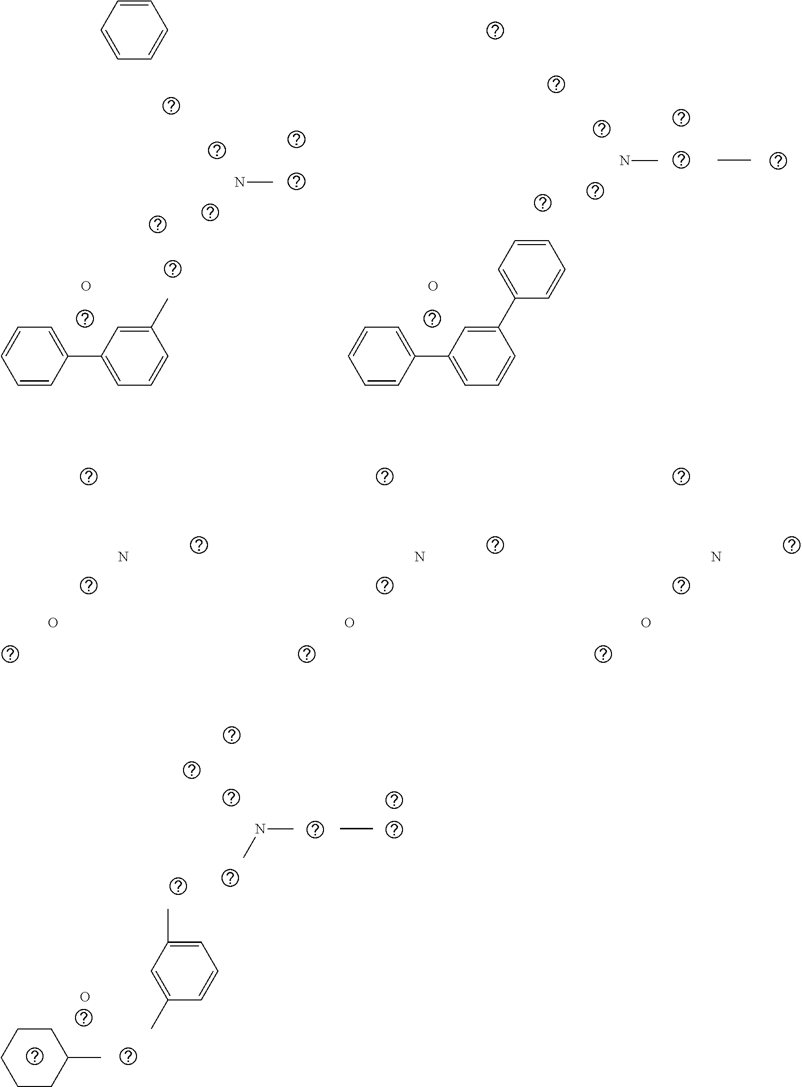

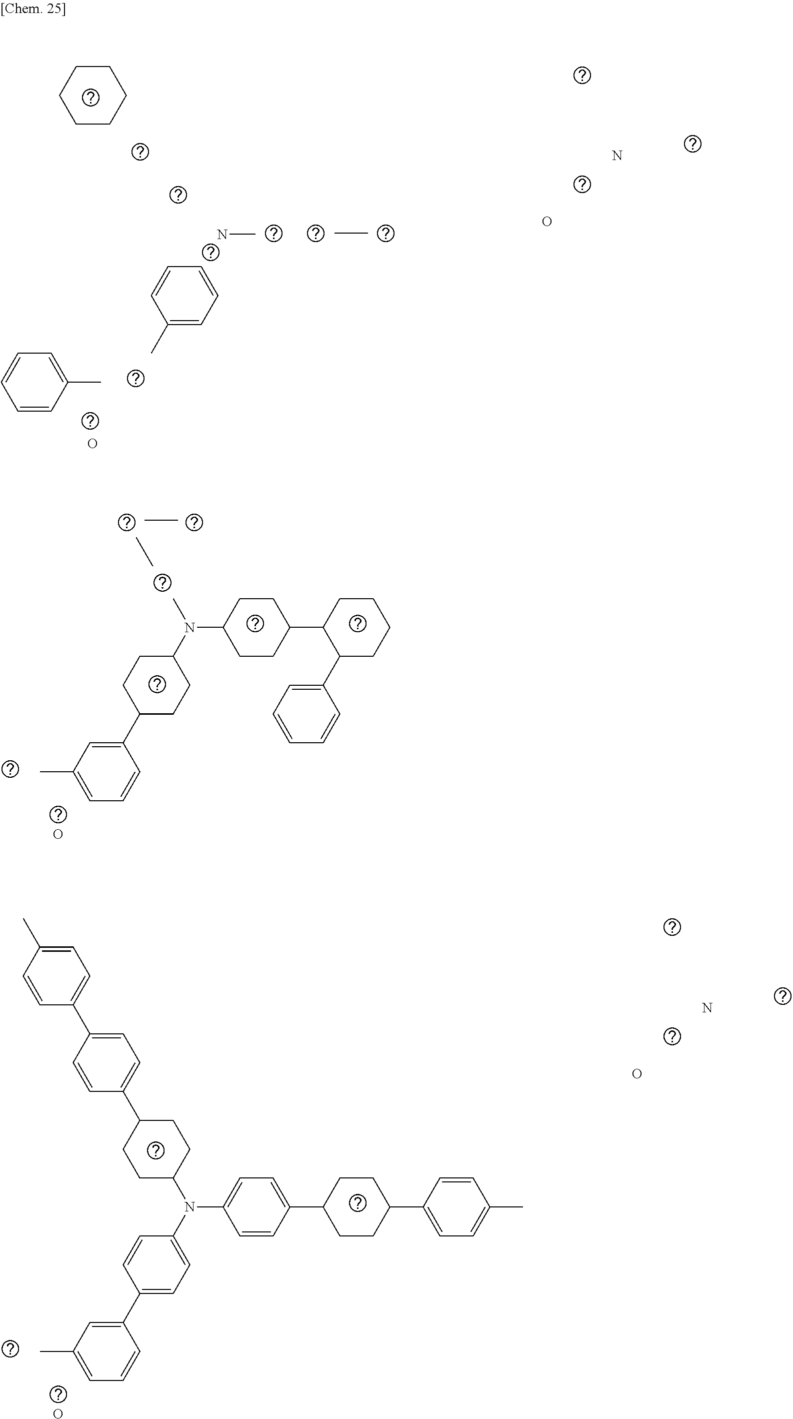

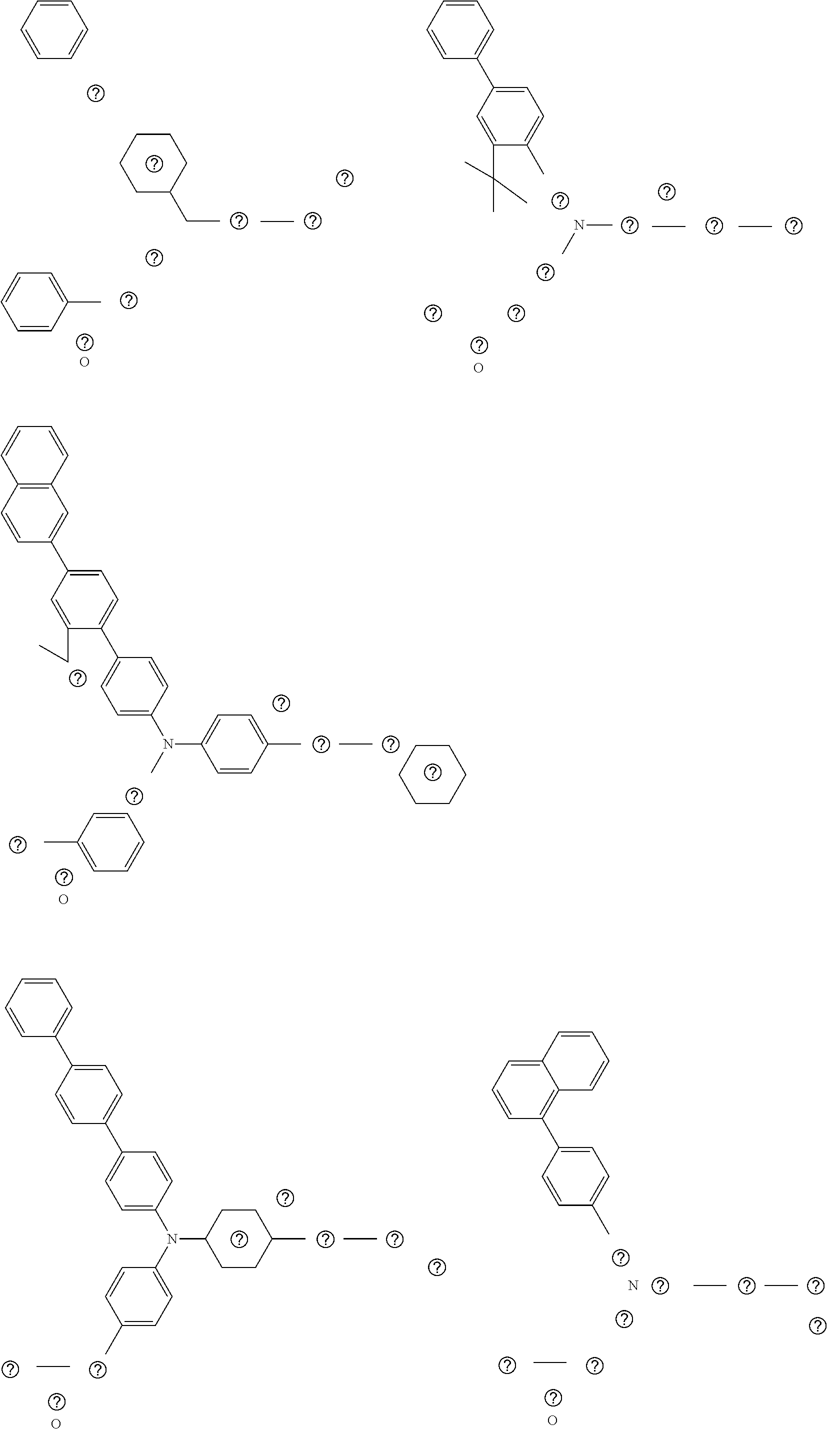

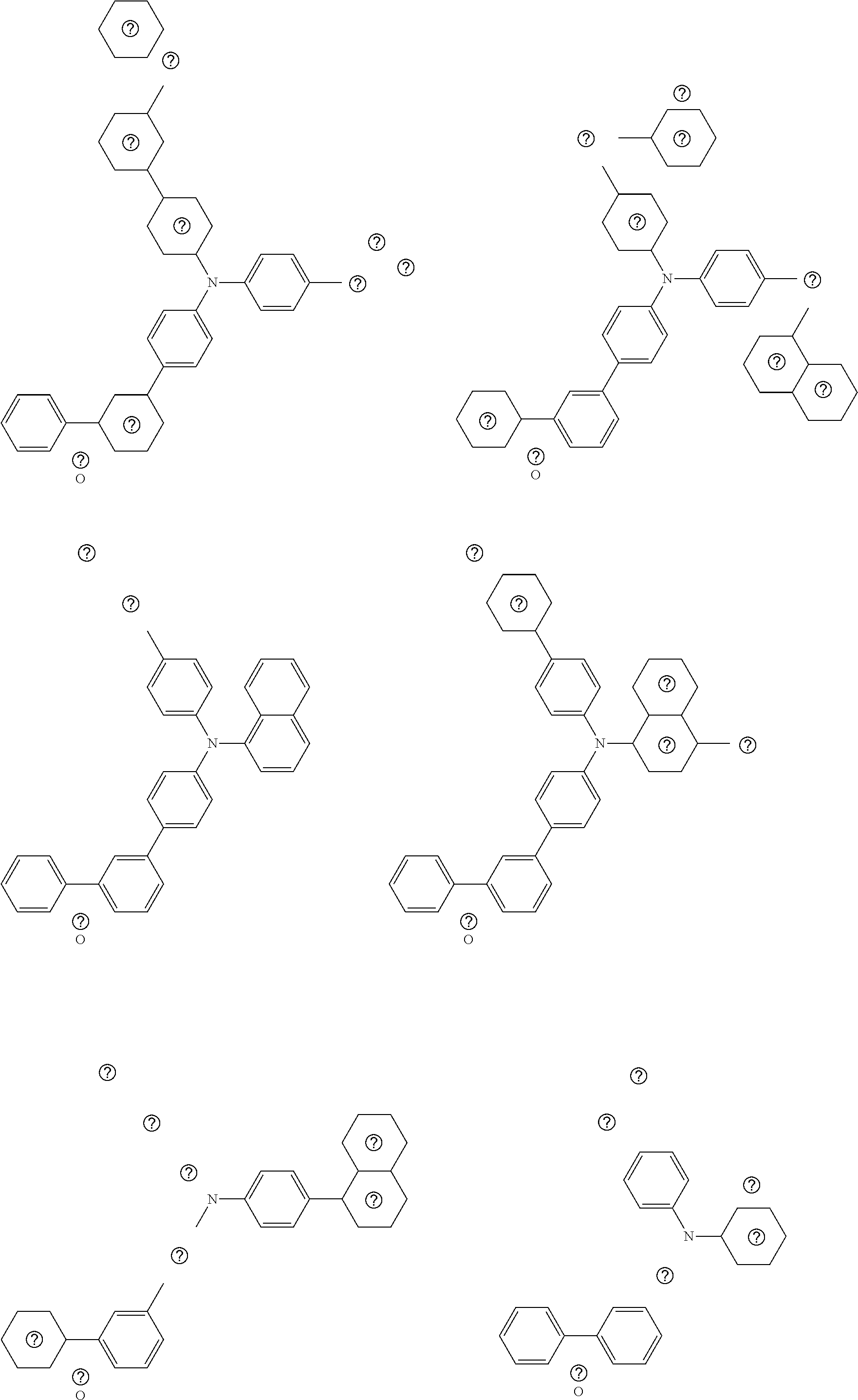

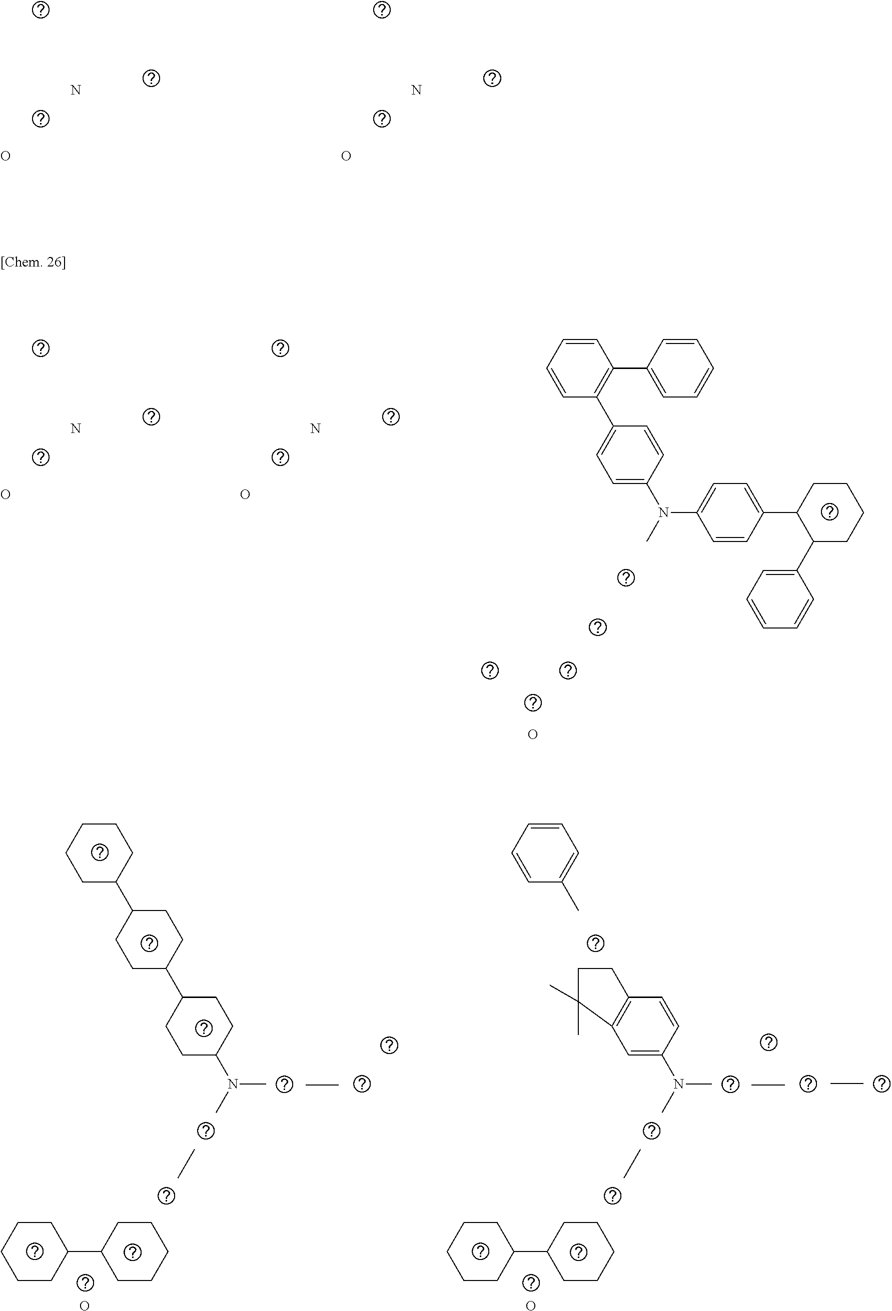

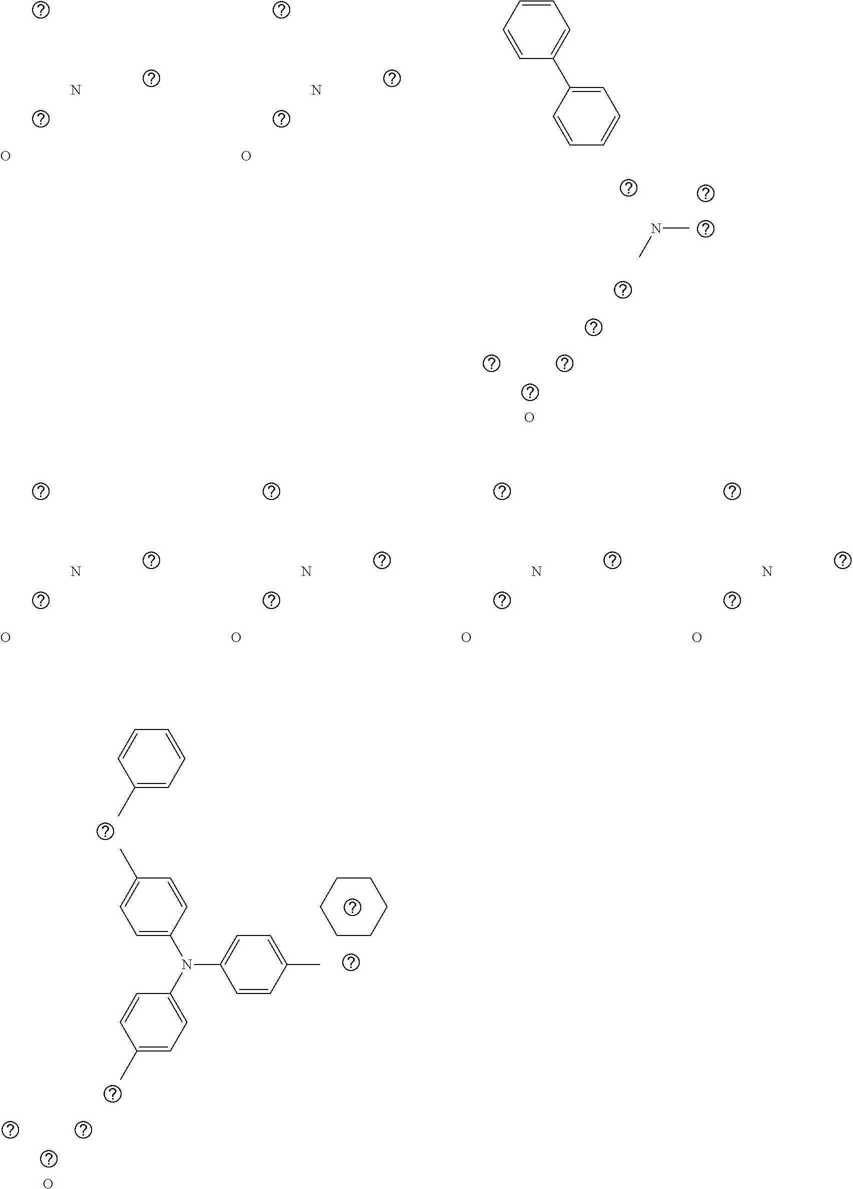

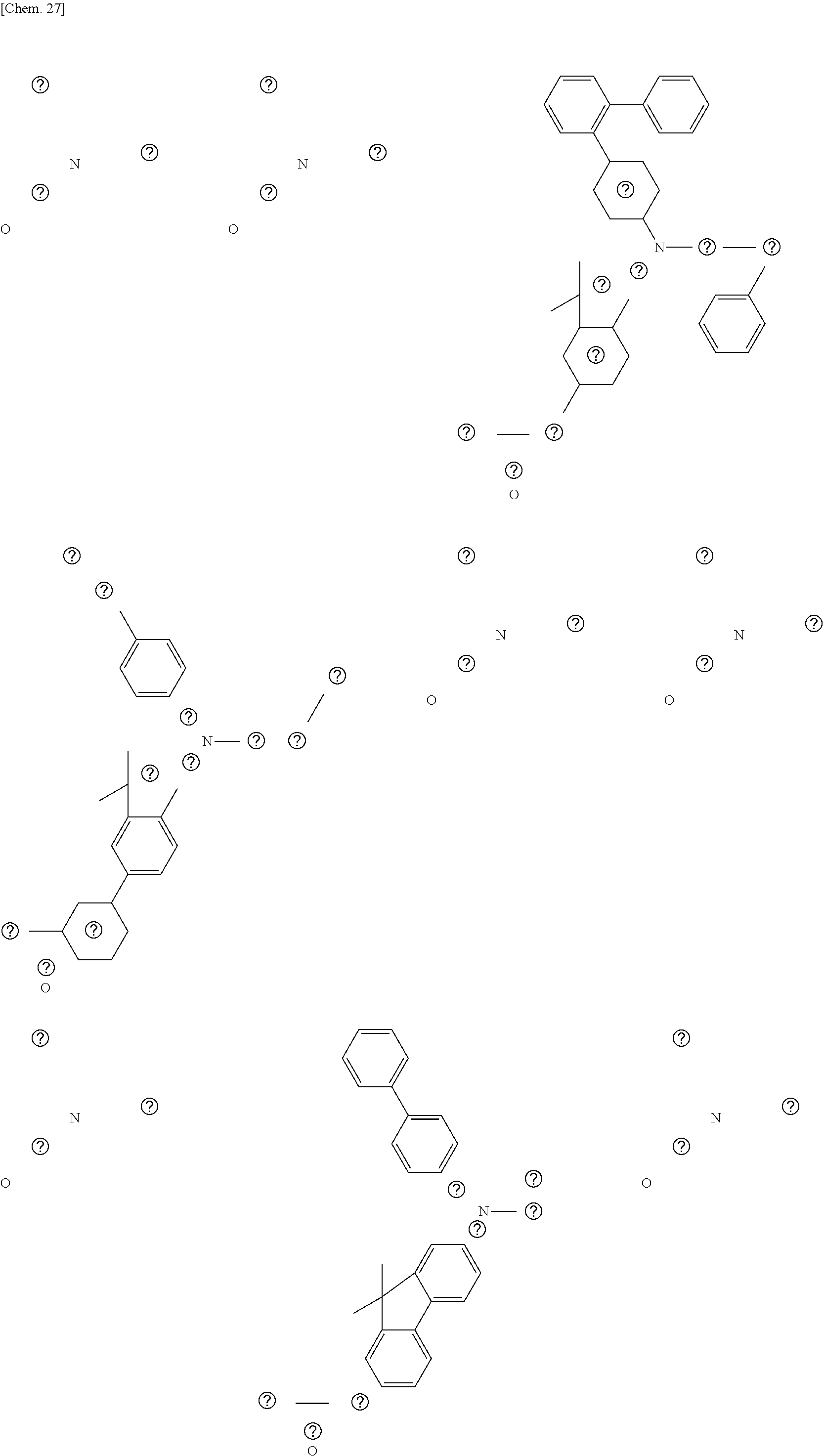

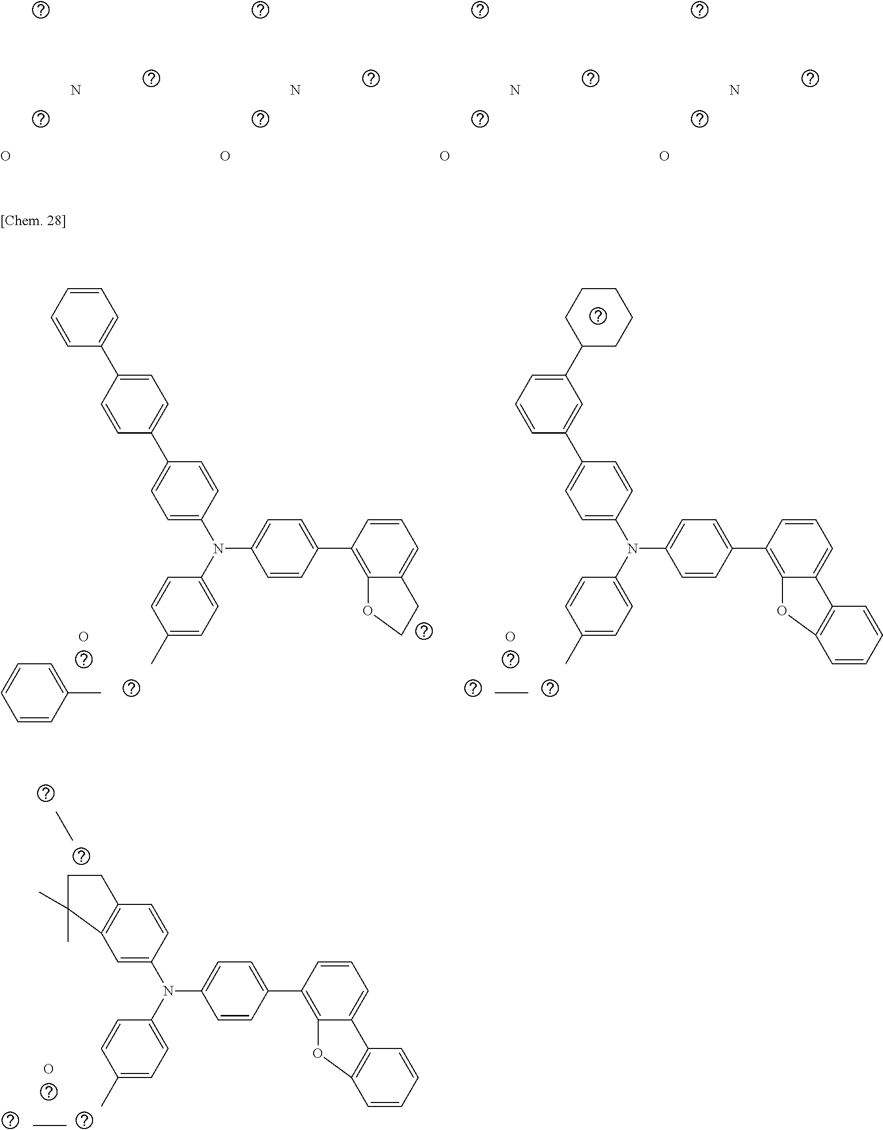

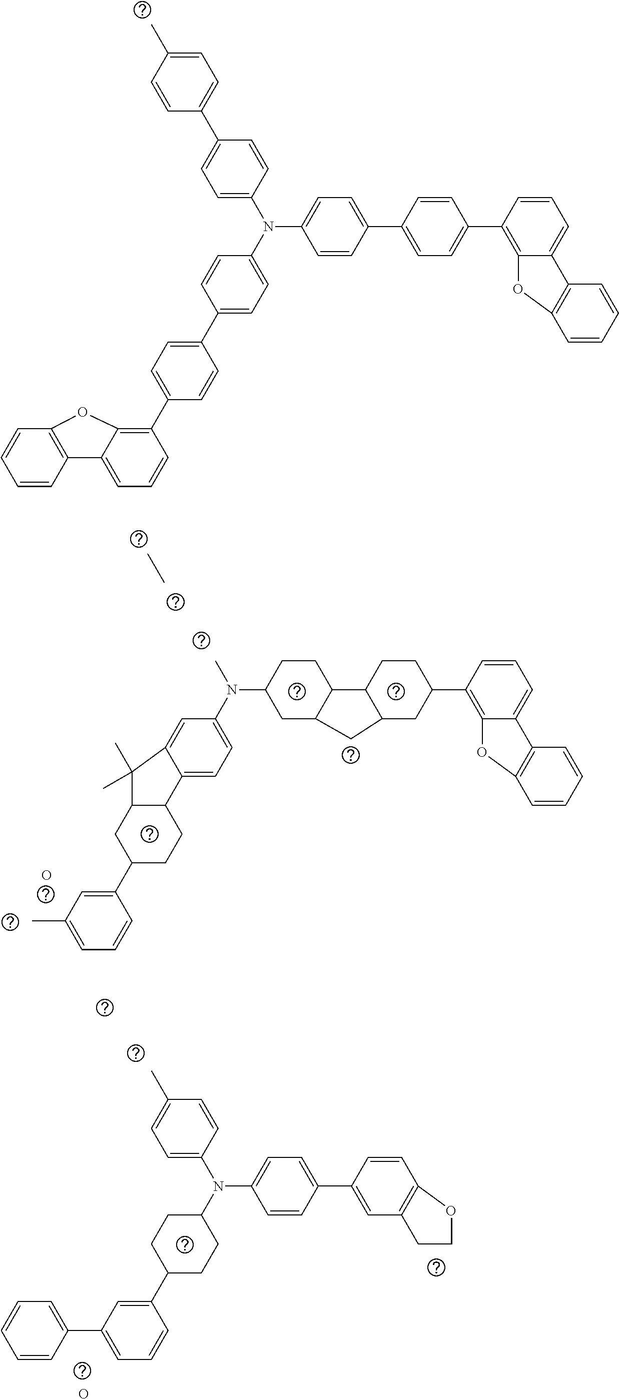

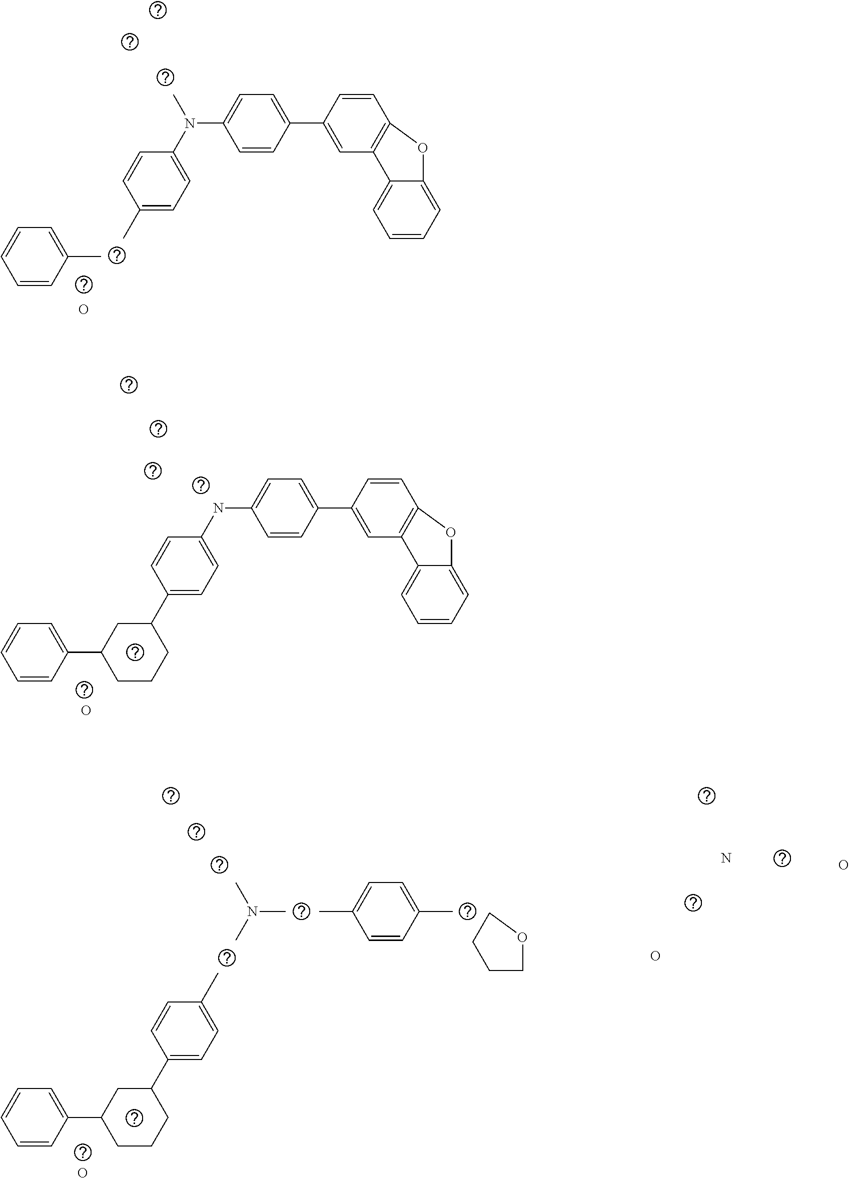

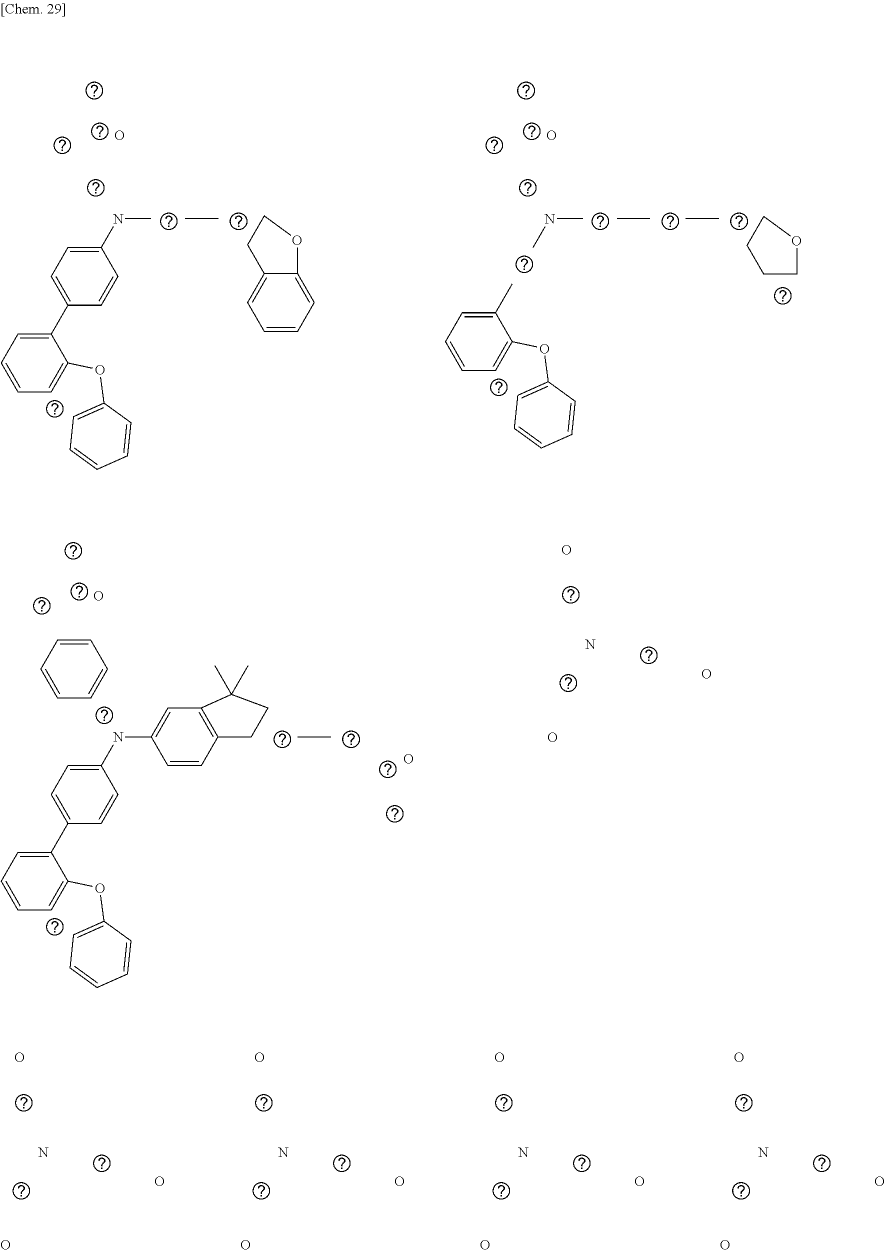

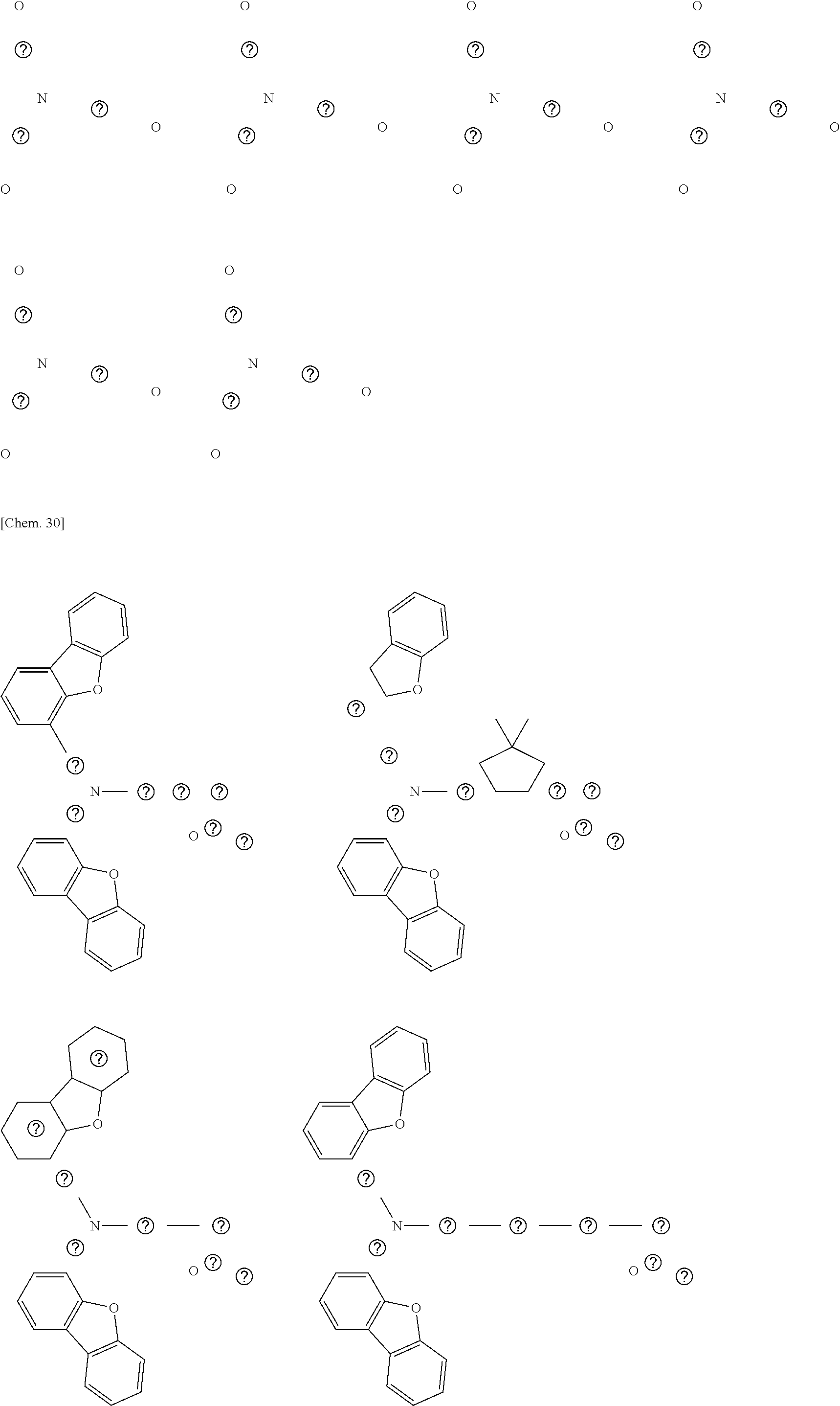

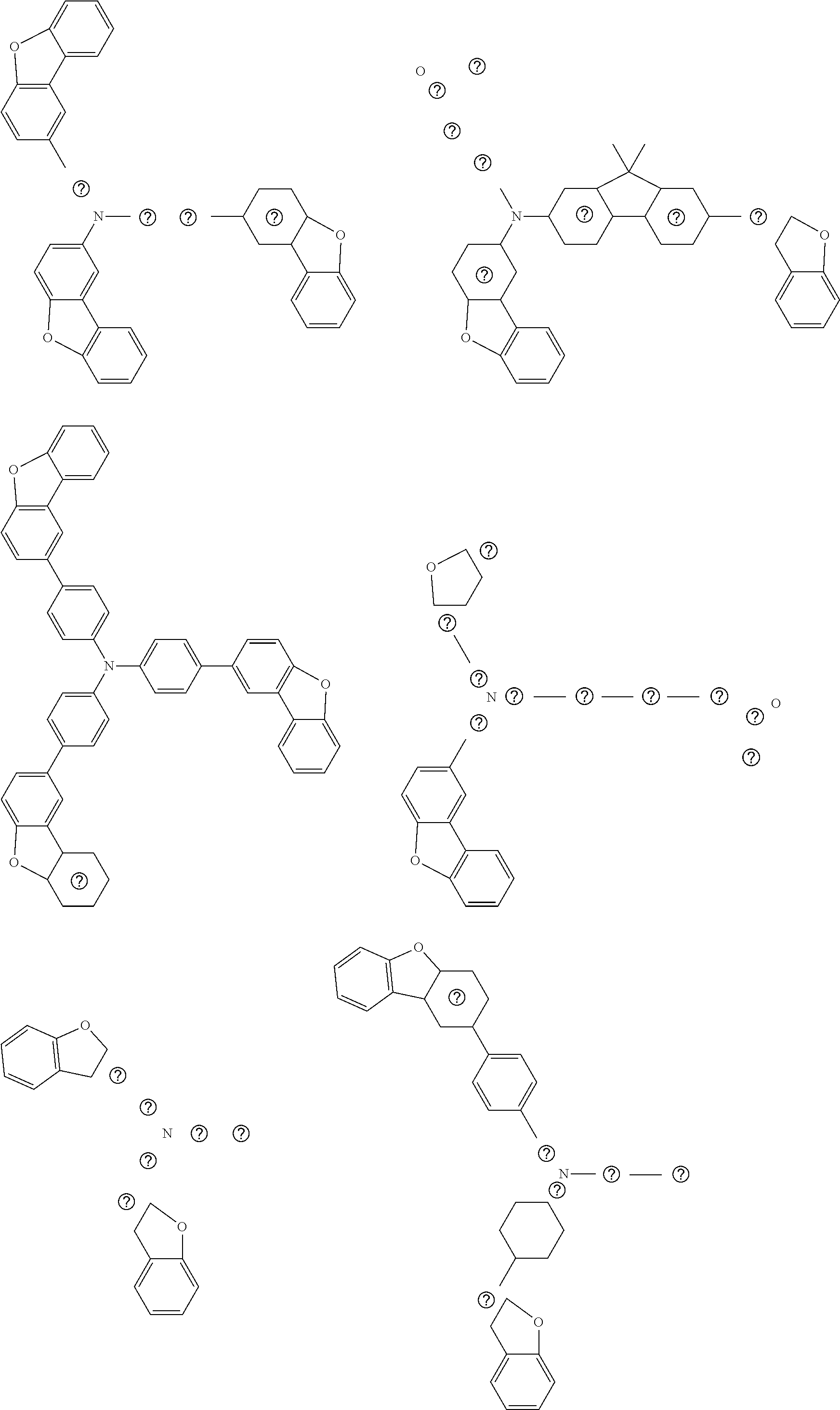

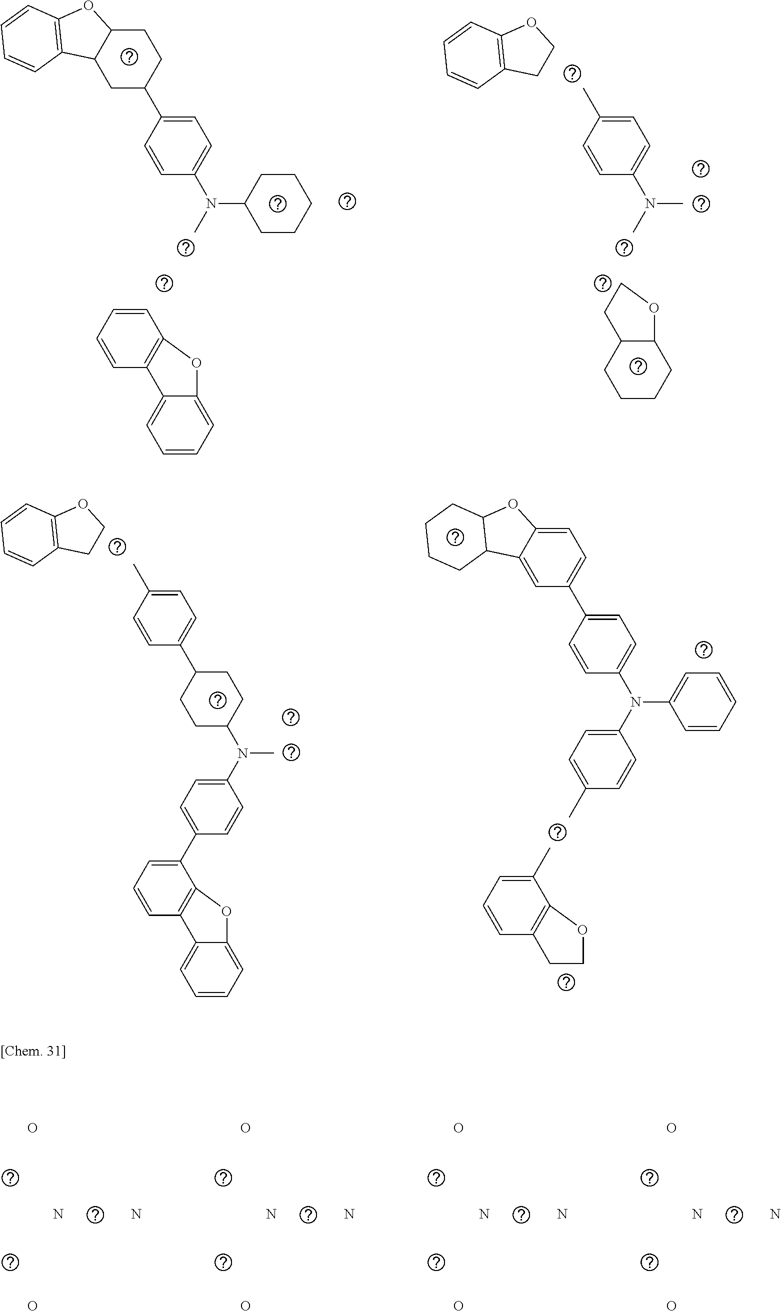

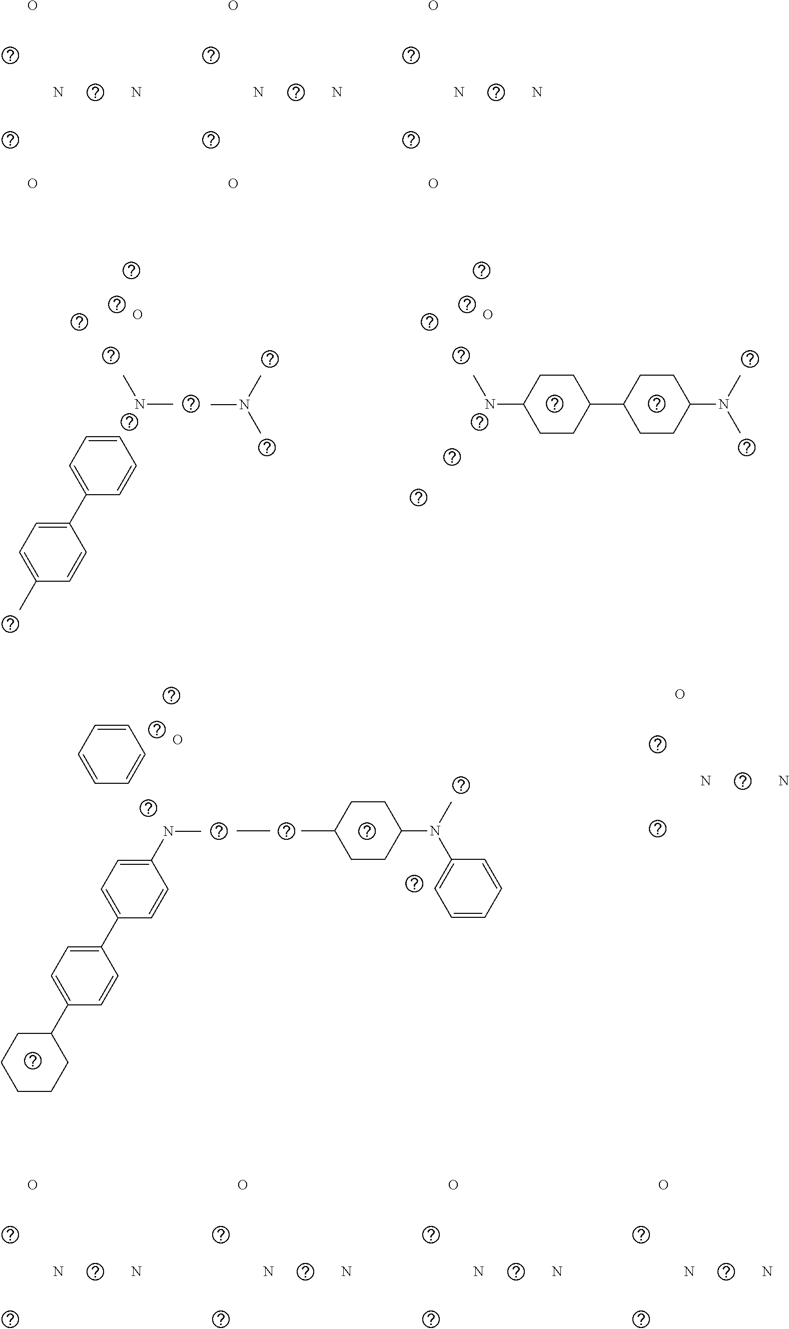

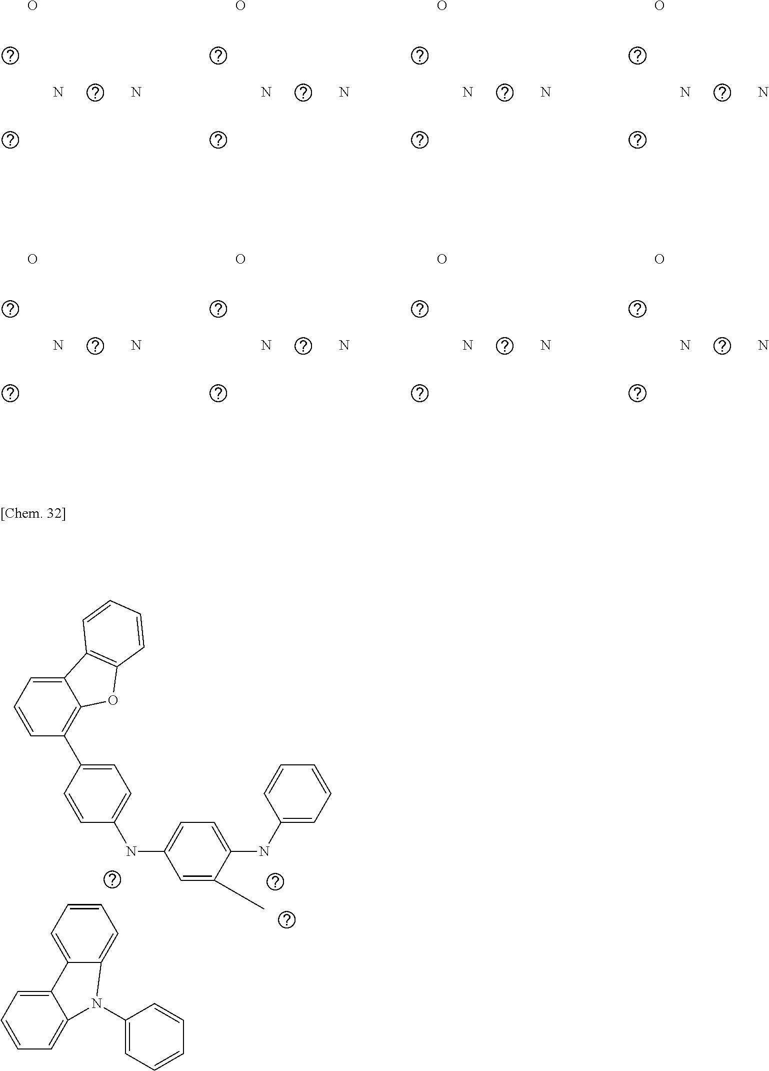

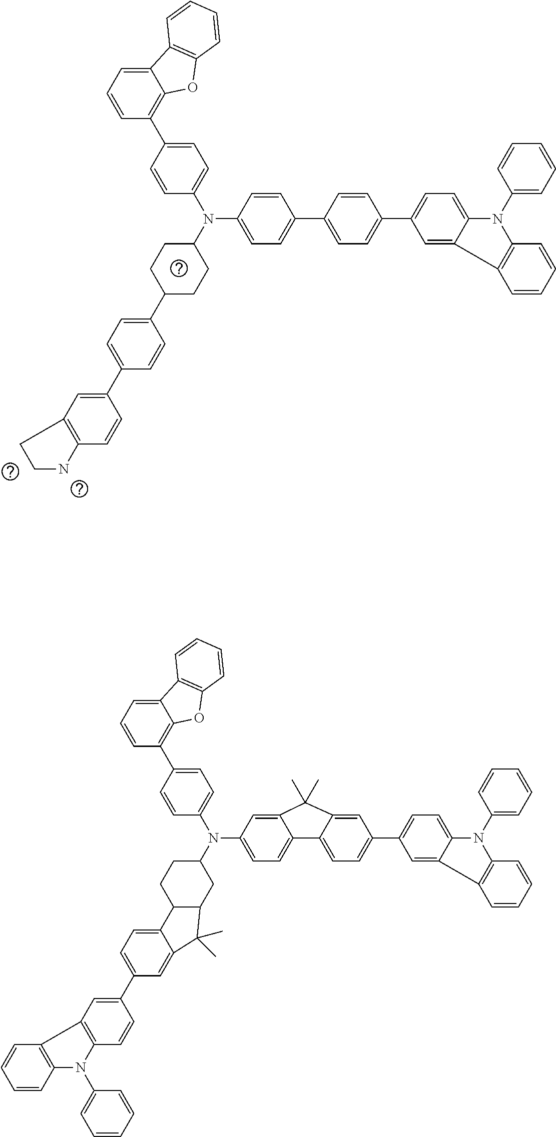

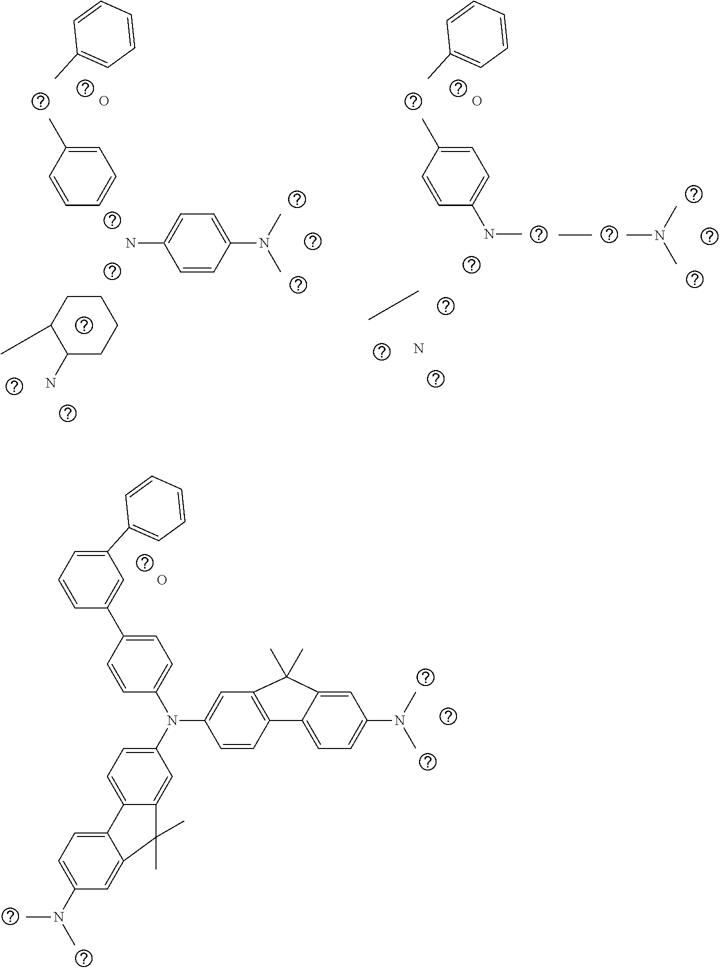

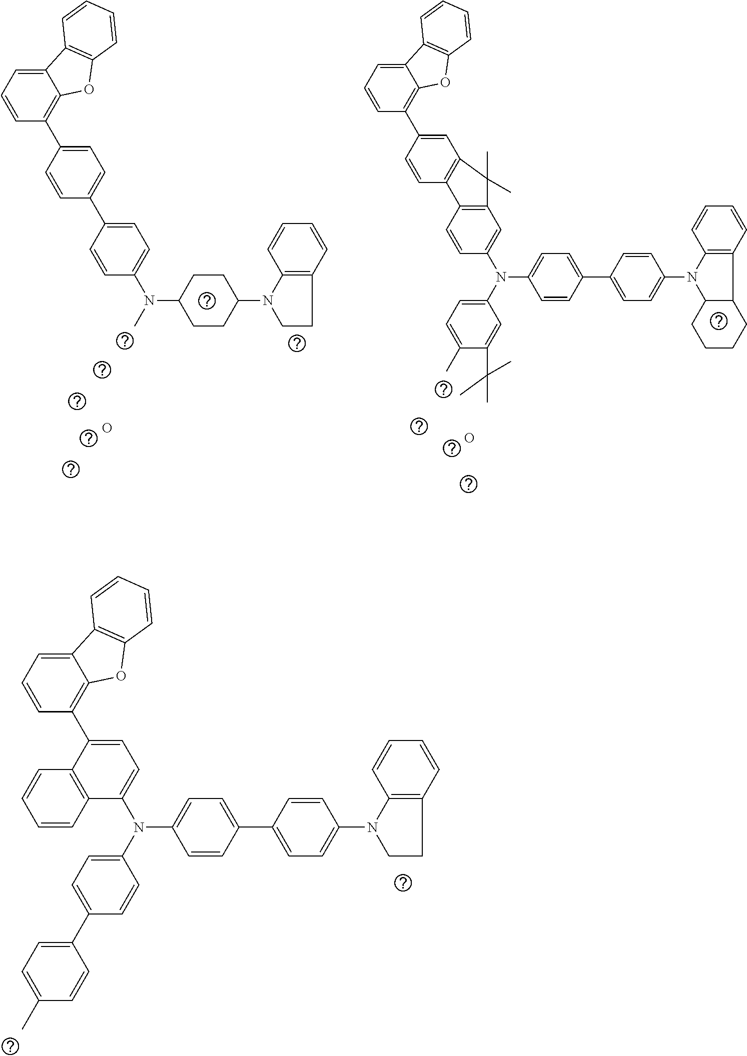

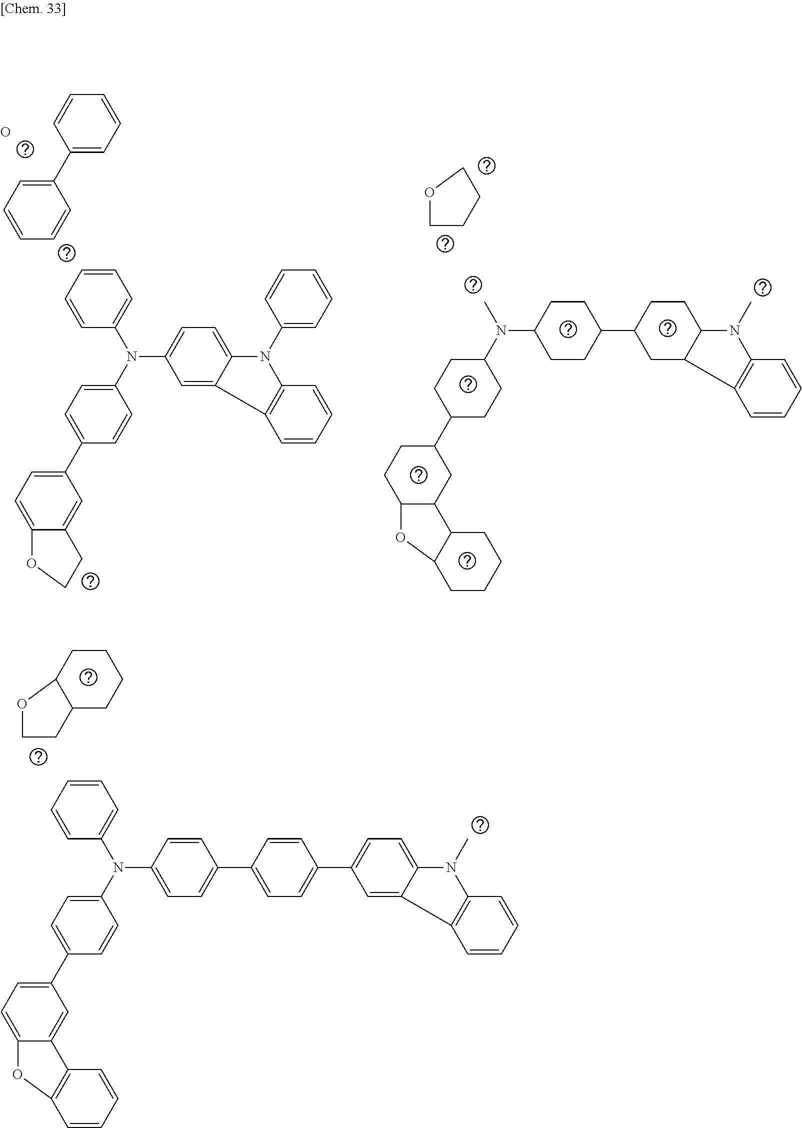

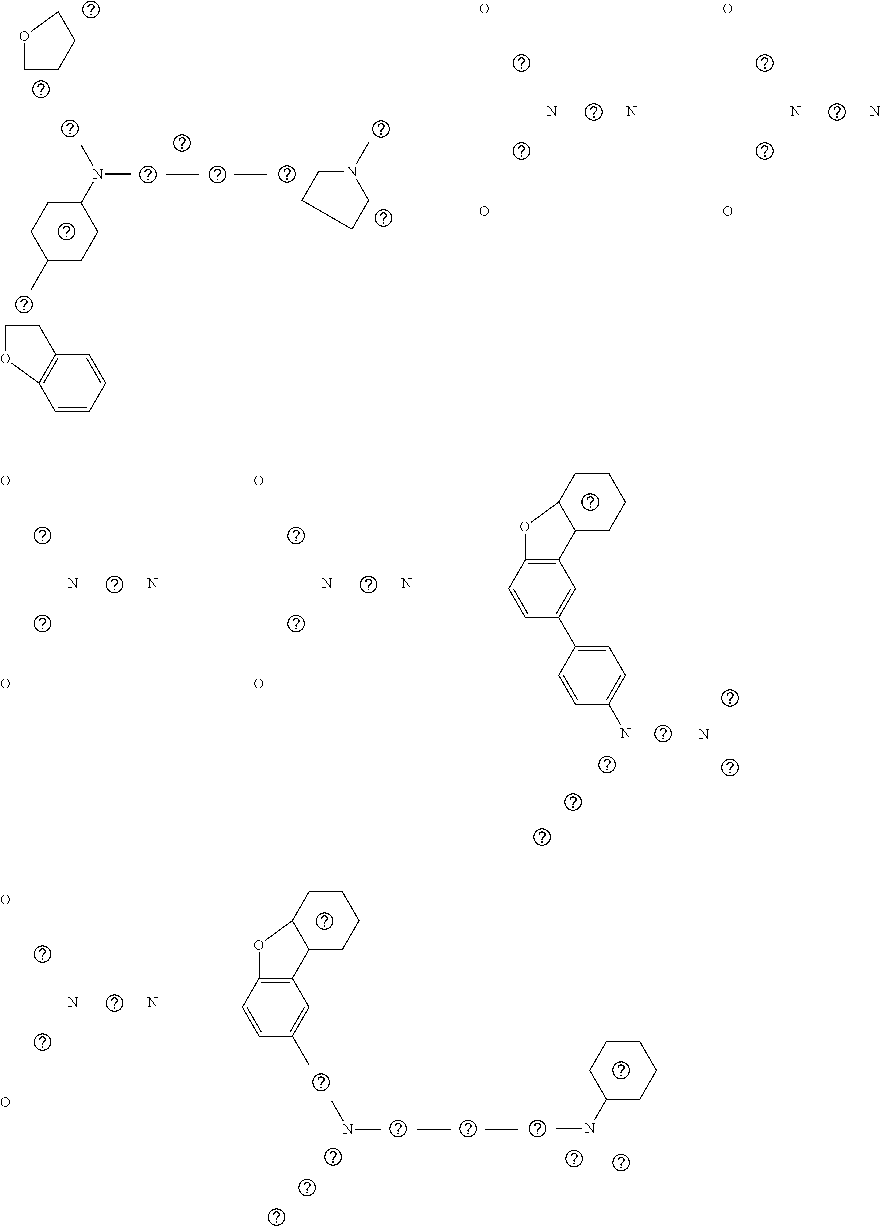

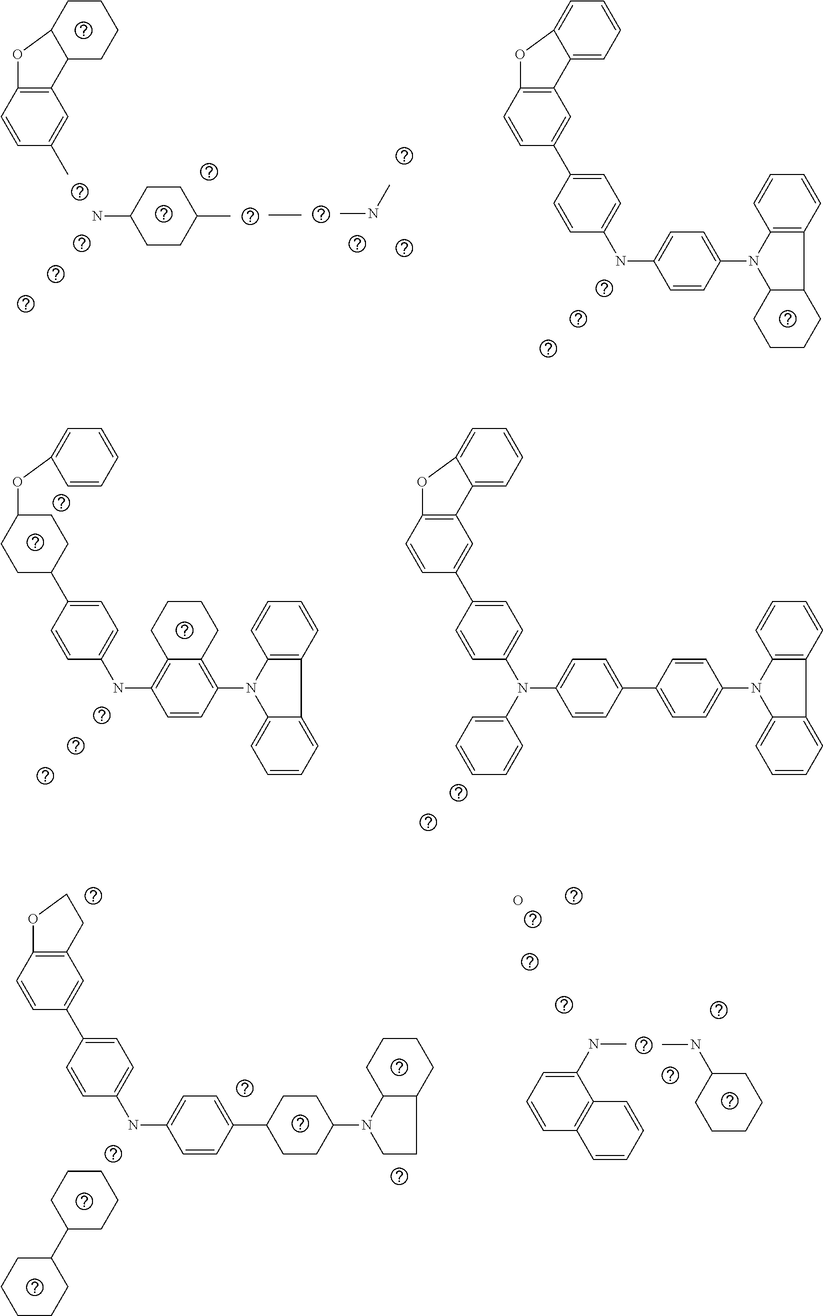

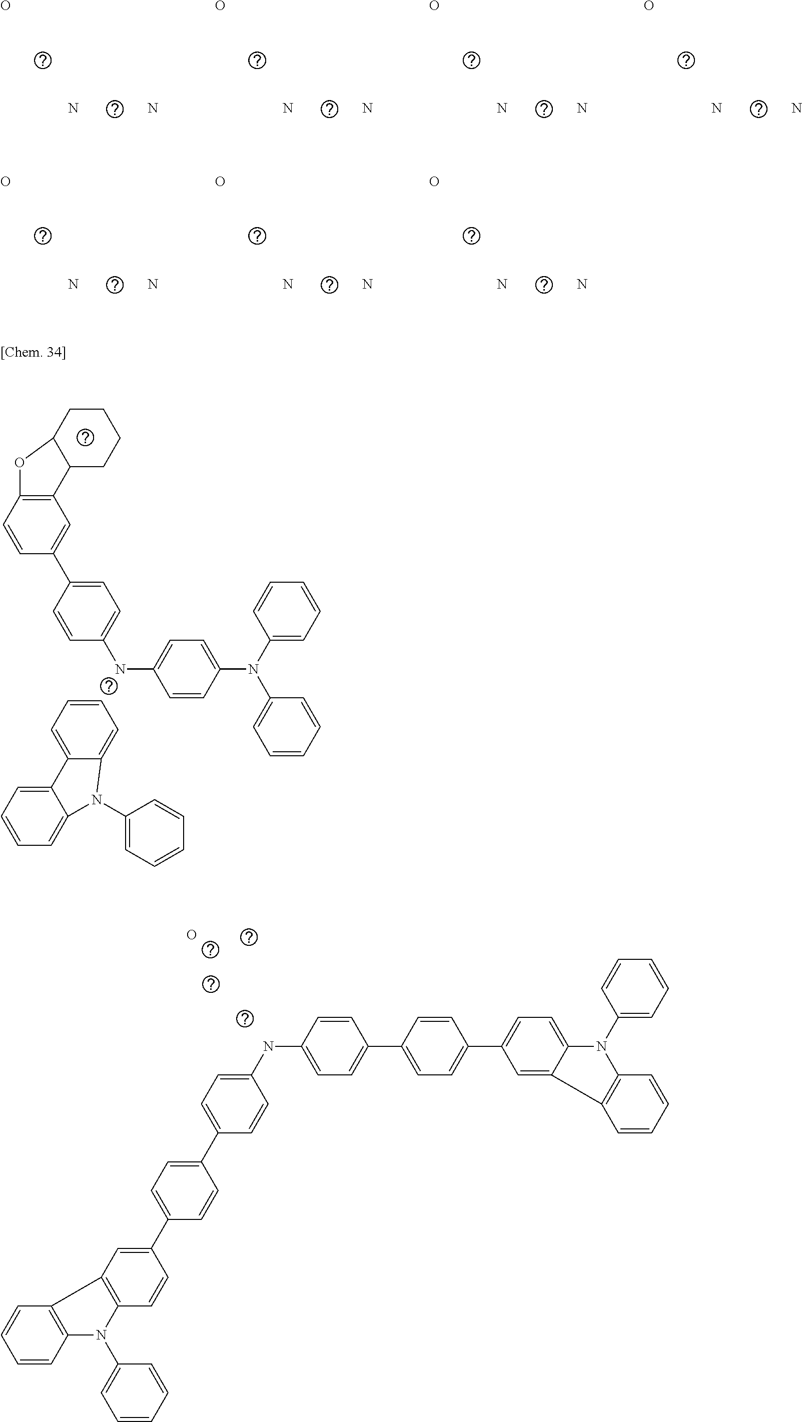

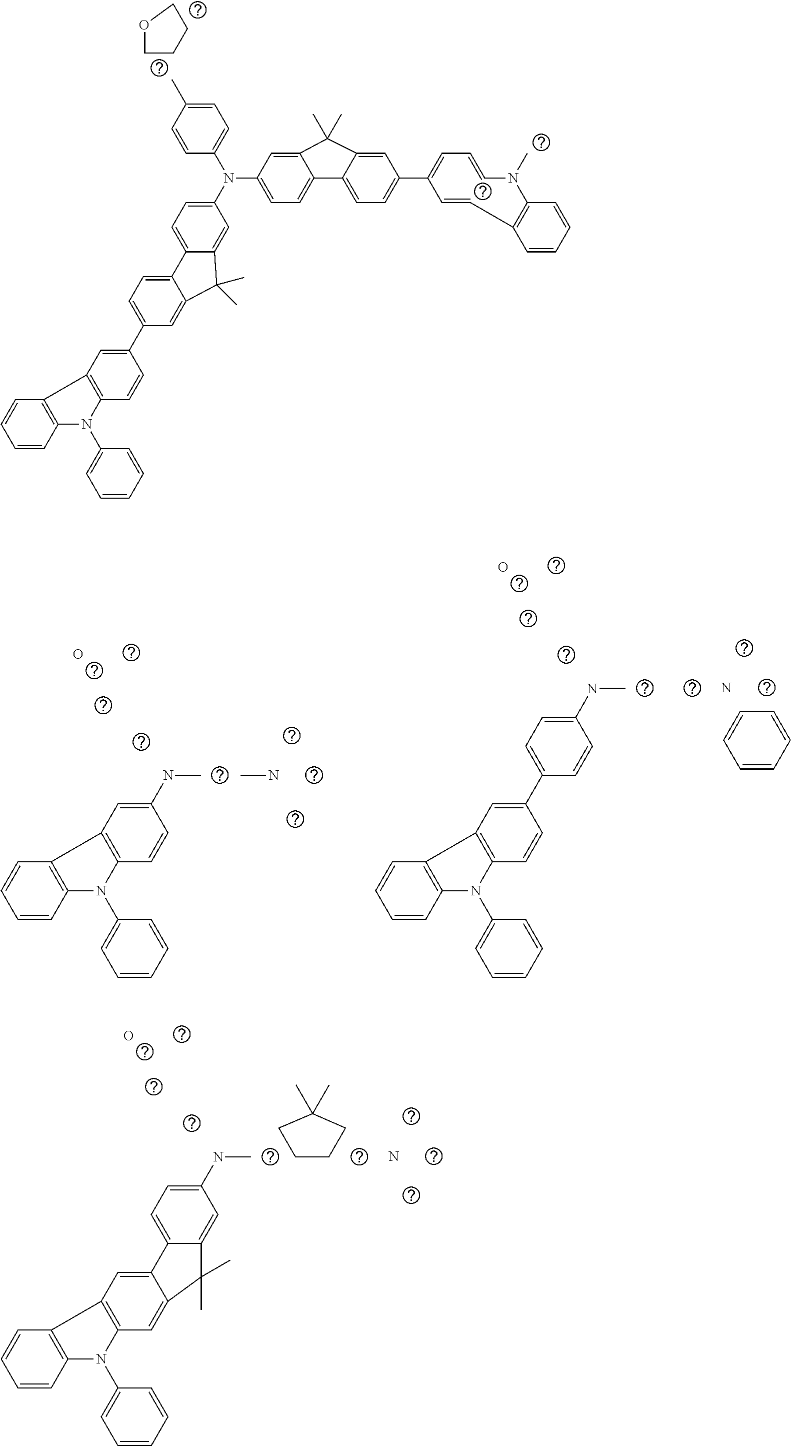

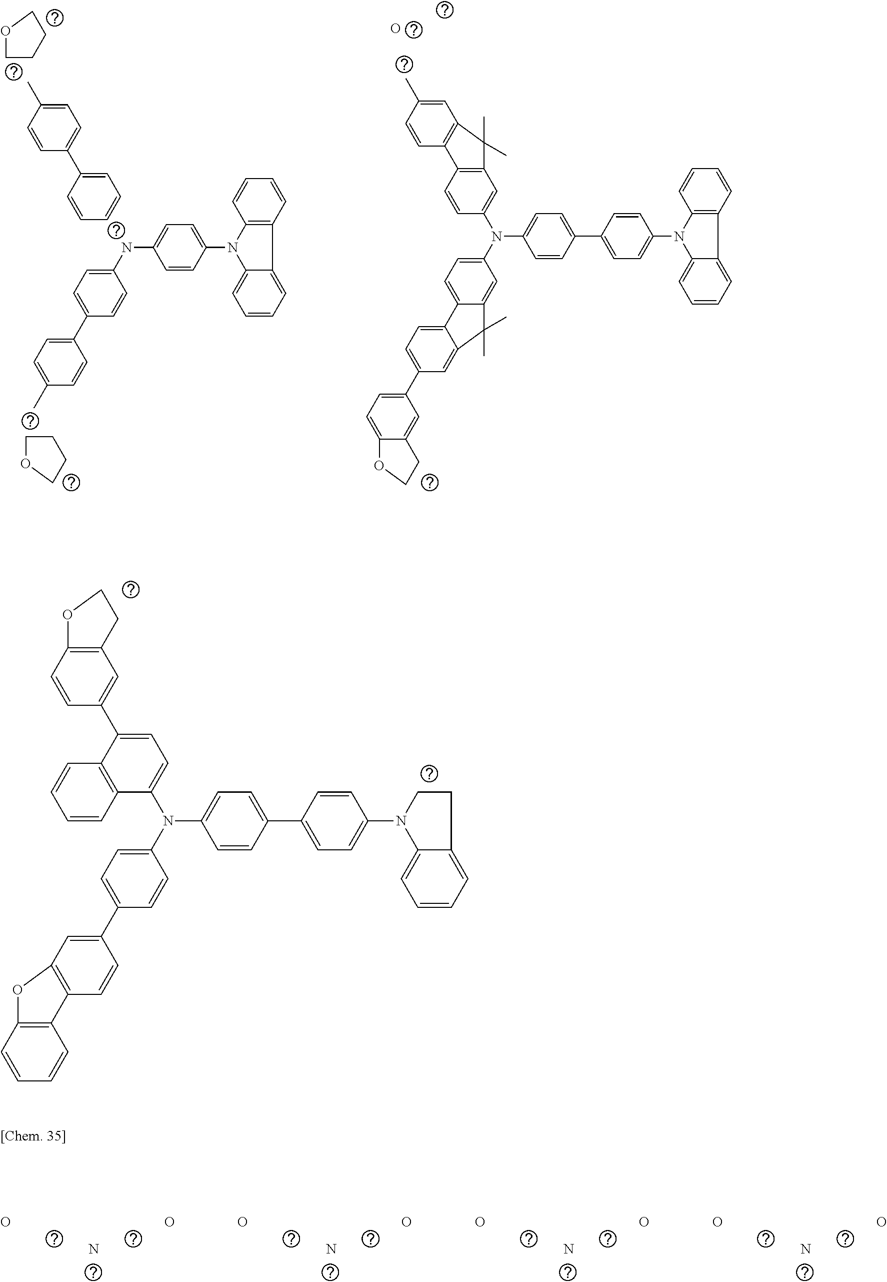

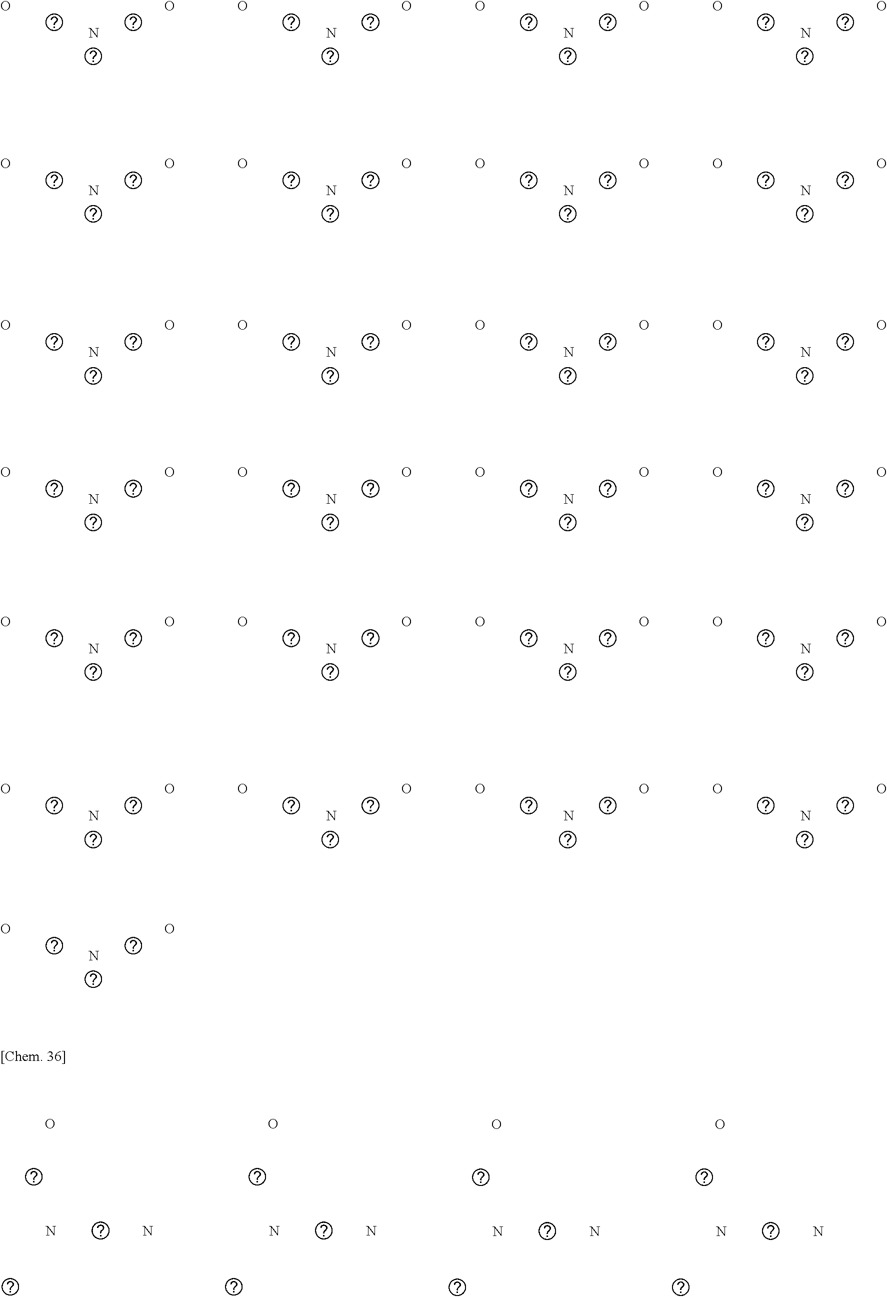

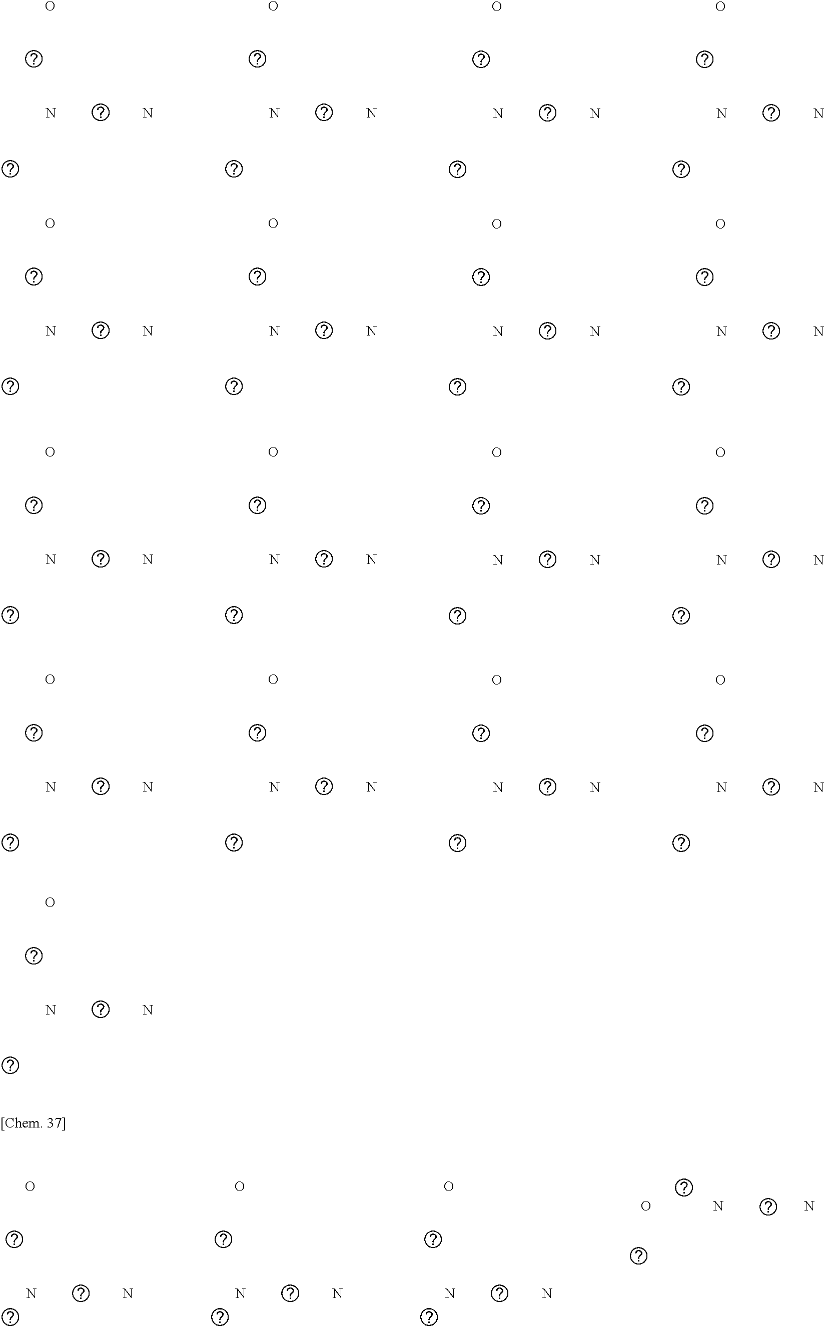

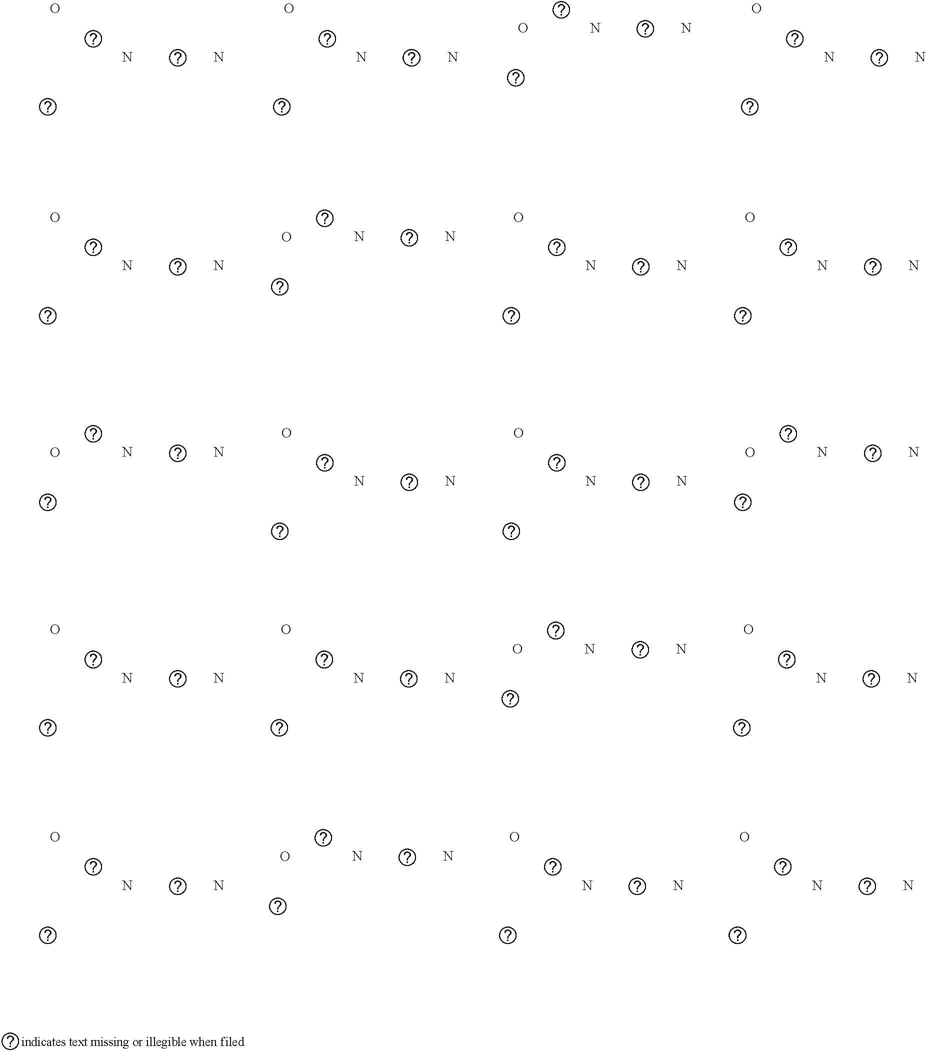

In addition, each of the compounds represented by the general formulae (1) and (2) in the hole transporting layer is not limited to one kind. In other words, the first hole transporting layer may contain a plurality of compounds each represented by the general formula (1), and the second hole transporting layer may contain a plurality of compounds each represented by the general formula (2).

In the present invention, the hole transporting layer has the first hole transporting layer and the second hole transporting layer in the stated order from the side of the anode, the first hole transporting layer has the amino compound represented by the general formula (1), and the second hole transporting layer contains the compound represented by the general formula (2).

In the present invention, the compound represented by the general formula (1) preferably has 4 or less nitrogen atoms and a molecular weight of 300 or more and 1,500 or less.

With such construction, the compound undergoes no thermal decomposition at the time of its vapor deposition, and hence a stable thin film having a high Tg is obtained. That is, the thin film can be formed by a vapor deposition method.

Here, a molecular weight of less than 300 is not preferred because the Tg reduces and hence the thin film lacks stability. On the other hand, a molecular weight in excess of 1,500 is not preferred because decomposition due to heat at the time of the vapor deposition is apt to occur.

It should be noted that a polymer material can also be suitably used as the compound represented by the general formula (1). In this case, an application method is preferably employed, and hence the compound can be used without any limitation on an upper limit for its molecular weight.

The organic electroluminescence device of the present invention preferably satisfies the following conditions (26) to (34).

(26) The hole transporting layer is joined to the light emitting layer.

Specifically, the second hole transporting layer is preferably joined to the light emitting layer.

(27) The light emitting material is a metal complex compound containing a metal selected from Ir, Pt, Os, Cu, Ru, Re, and Au.

When such metal complex compound is used as a light emitting material, the quantum yield of light emission is high, and the external quantum efficiency of the light emitting device can be additionally improved.

In particular, the material is preferably an iridium complex, an osmium complex, or a platinum complex, more preferably an iridium complex or a platinum complex, most preferably an ortho-metalated iridium complex.

(28) In the light emitting material, a central metal atom and a carbon atom contained in a ligand are bonded through an ortho-metal bond.

With such construction, the quantum yield of light emission can be additionally improved.

Preferred examples of the ortho-metalated metal complex include the following iridium complexes.

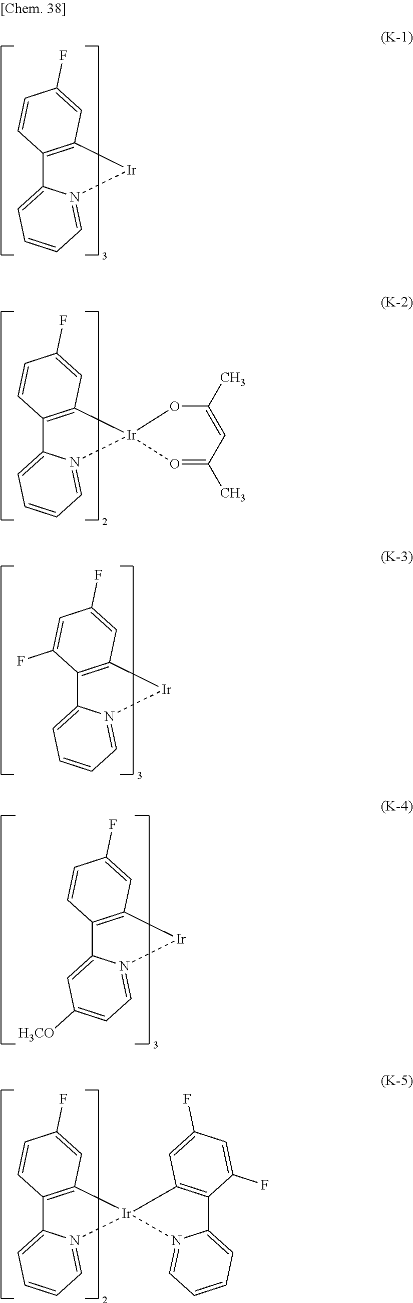

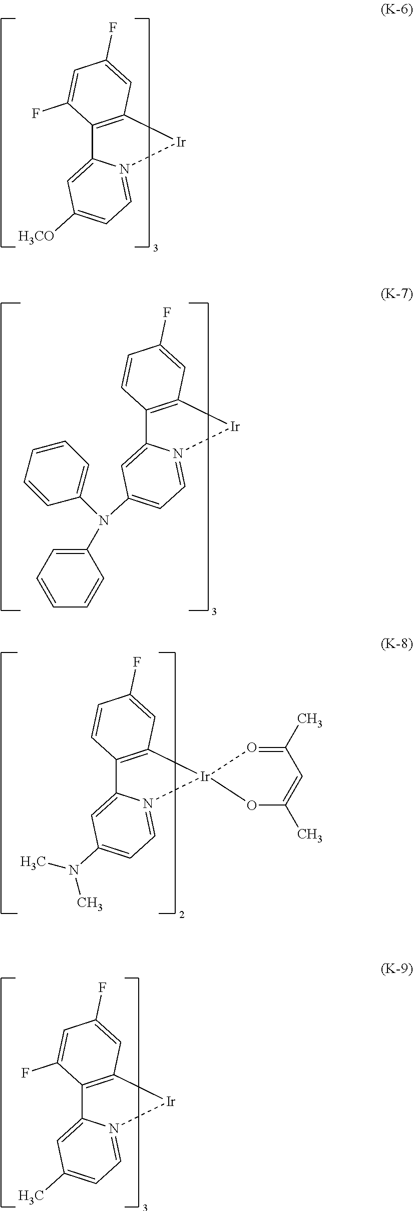

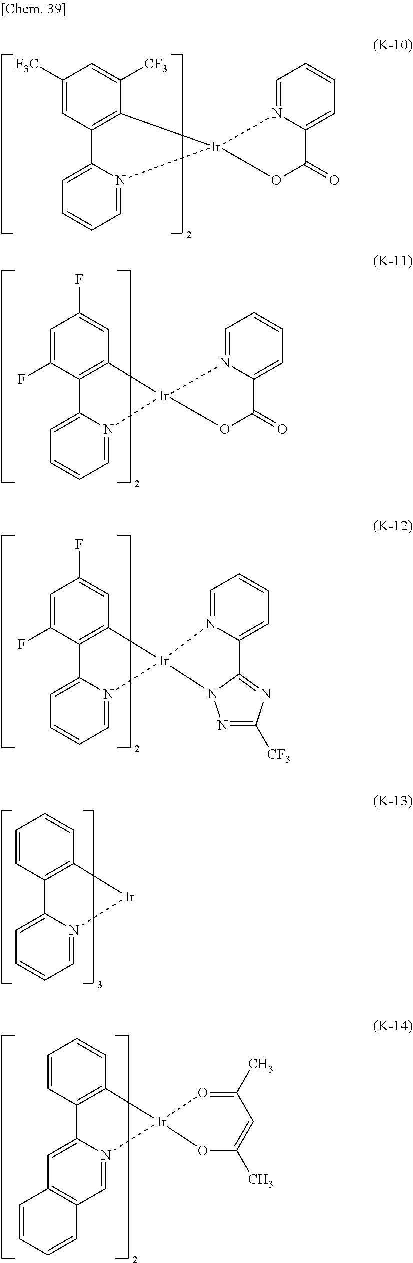

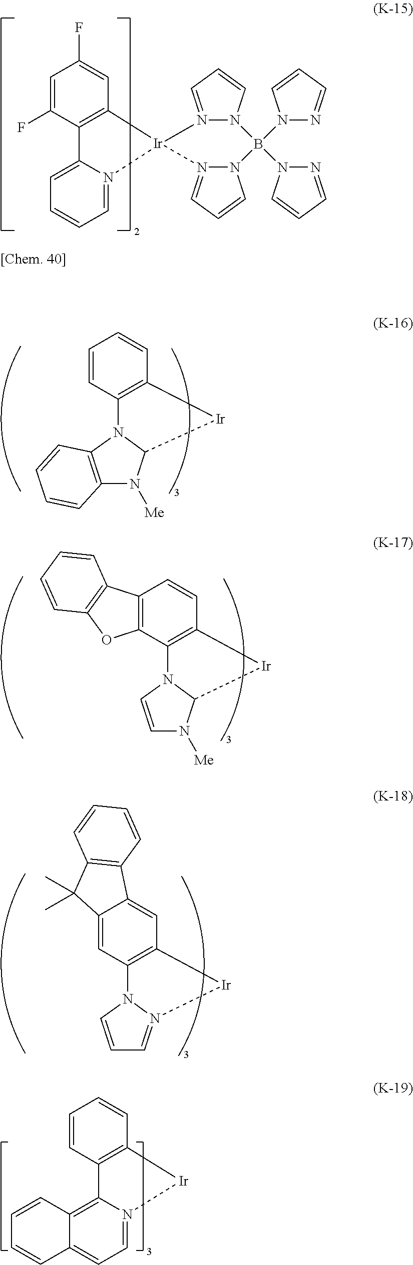

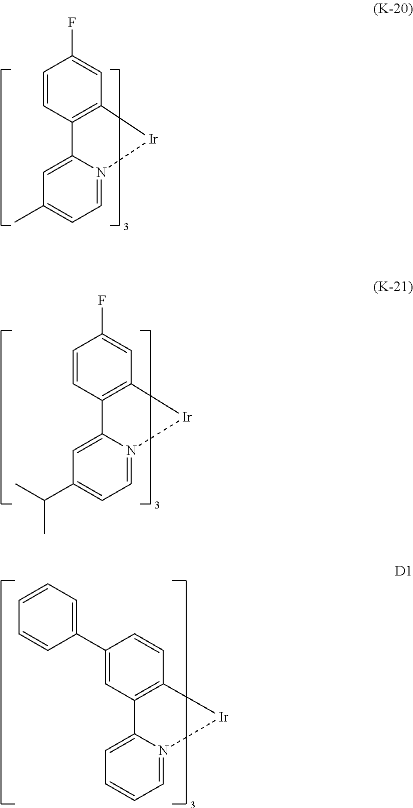

(29) The host material has an excited triplet energy gap of 2.0 eV or more and 3.2 eV or less.

With such construction, energy can be effectively transferred to the light emitting material.

Here, the excited triplet energy gap Eg(T) can be specified on the basis of, for example, a light emission spectrum as described below.

That is, a material to be measured is dissolved in an EPA solvent (containing diethyl ether, isopentane, and ethanol at a volume ratio of 5:5:2) at 10 μmol/L so that a sample for phosphorescence measurement may be prepared.

Then, the sample for phosphorescence measurement is charged into a quartz cell, cooled to 77 K, and irradiated with excitation light. Then, the wavelength of radiated phosphorescence is measured.

A tangent is drawn to the rise-up of the resultant phosphorescence spectrum at shorter wavelengths, and then a wavelength value for a point of intersection of the tangent and a baseline is converted into energy. The resultant value is defined as the excited triplet energy gap Eg(T).

It should be noted that a commercially available measuring apparatus F-4500 (manufactured by Hitachi, Ltd.) may be used in the measurement.

(30) A reduction-causing dopant is added at an interfacial region between the cathode and the organic thin-film layer.

Examples of the reduction-causing dopant include at least one kind selected from an alkali metal, an alkali metal complex, an alkali metal compound, an alkaline earth metal, an alkaline earth metal complex, an alkaline earth metal compound, a rare earth metal, a rare earth metal complex, and a rare earth metal compound.

Examples of the alkali metal include Na (work function: 2.36 eV), K (work function: 2.28 eV), Rb (work function: 2.16 eV), and Cs (work function: 1.95 eV). Of those, an alkali metal having a work function of 2.9 eV or less is particularly preferred. Of those, preferred are K, Rb, and Cs, more preferred are Rb and Cs, and most preferred is Cs.

Examples of the alkaline earth metal include Ca (work function: 2.9 eV), Sr (work function: 2.0 to 2.5 eV), and Ba (work function: 2.52 eV). Of those, an alkaline earth metal having a work function of 2.9 eV or less is particularly preferred.

Examples of the rare earth metal include Sc, Y, Ce, Tb, and Yb. Of those, a rare earth metal having a work function of 2.9 eV or less is particularly preferred.

Each of the above-mentioned metals has a particularly high reductive ability, and hence the addition thereof in a relatively small amount to an electron injecting region can improve the luminous brightness and lengthen the lifetime in the organic EL device.

Examples of the alkali metal compound include an alkali oxide such as Li₂O, Cs₂O, or K₂O, and an alkali halide such as LiF, NaF, CsF, or KF. Of those, an alkali oxide or alkali fluoride such as LiF, Li₂O, or NaF is preferred.

Examples of the alkaline earth metal compound include BaO, SrO, CaO, and mixtures thereof such as BaₓSr₁₋ₓO (0<x<1) and BaₓCa₁₋ₓO (0<x<1). Of those, BaO, SrO, and CaO are preferred.

Examples of the rare earth metal compound include YbF₃, ScF₃, ScO₃, Y₂O₃, Ce₂O₃, GdF₃, and TbF₃. Of those, YbF₃, ScF₃, and TbF₃ are preferred.

The alkali metal complex, alkaline earth metal complex, and rare earth metal complex are not particularly limited as long as the complexes each contain, as a metal ion, at least one of alkali metal ions, alkaline earth metal ions, and rare earth metal ions. Meanwhile, preferred examples of the ligand include, but are not limited to, quinolinol, benzoquinolinol, acridinol, phenanthridinol, hydroxyphenyloxazole, hydroxyphenylthiazole, hydroxydiaryloxadiazole, hydroxydiarylthiadiazole, hydroxyphenylpyridine, hydroxyphenylbenzimidazole, hydroxybenzotriazole, hydroxyfluborane, bipyridyl, phenanthroline, phthalocyanine, porphyrin, cyclopentadiene, β-diketones, azomethines, and derivatives thereof.

For the addition form of the reduction-causing dopant, it is preferred that the reduction-causing dopant be formed into a shape of a layer or an island in the interfacial region. A preferred forming method is a method in which an organic substance as a light emitting material or an electron injecting material for forming the interfacial region is deposited at the same time as the reduction-causing dopant is deposited by a resistance heating deposition method, thereby dispersing the reduction-causing dopant in the organic substance. The disperse concentration by molar ratio of the organic substance to the reduction-causing dopant is 100:1 to 1:100, preferably 5:1 to 1:5.

When the reduction-causing dopant is formed into the shape of a layer, the light emitting material or electron injecting material which serves as an organic layer in the interface is formed into the shape of a layer. After that, the reduction-causing dopant is solely deposited by the resistance heating deposition method to form a layer preferably having a thickness of 0.1 to 15 nm.

In the case where the reduction-causing dopant is formed into the shape of an island, the light emitting material or electron injecting material which serves as an organic layer in the interface is formed into the shape of an island. After that, the reduction-causing dopant is solely deposited by the resistance heating deposition method to form an island preferably having a thickness of 0.05 to 1 nm.

In addition, a ratio “main component:reduction-causing dopant” between the main component and the reduction-causing dopant in the organic EL device of the present invention is preferably 5:1 to 1:5, more preferably 2:1 to 1:2 in terms of a molar ratio.

(31) The light emitting layer and the cathode have an electron injecting layer therebetween, and the electron injecting layer contains a nitrogen-containing ring derivative as a main component.

Here, the phrase “as a main component” means that the electron injecting layer contains at least 50 mass % of the nitrogen-containing ring derivative.

An aromatic heterocyclic compound containing one or more heteroatoms in any one of its molecules is preferably used as an electron transporting material used in the electron injecting layer, and the nitrogen-containing ring derivative is particularly preferred.

The nitrogen-containing ring derivative is preferably, for example, a nitrogen-containing ring derivative represented by the following formula (A).

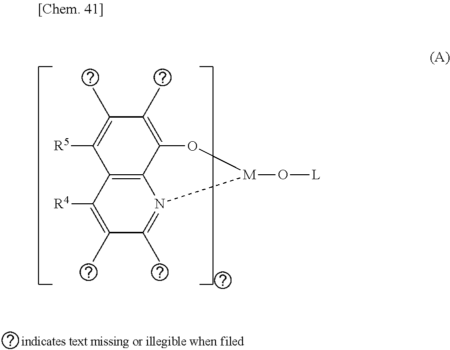

In the formula (A), R² to R⁷ each independently represent a hydrogen atom, a halogen atom, an oxy group, an amino group, or a hydrocarbon group having 1 to 40 carbon atoms, each of which may be substituted.

Examples of the halogen atom include fluorine and chlorine. Further, examples of the amino group that may be substituted include an alkylamino group, an arylamino group, an aralkylamino group, and the same examples as those of the above-mentioned amino group.

Examples of the hydrocarbon group having 1 to 40 carbon atoms include a substituted or unsubstituted alkyl group, alkenyl group, cycloalkyl group, alkoxy group, aryl group, heterocyclic group, aralkyl group, aryloxy group, and alkoxycarbonyl group. Examples of the alkyl group, the alkenyl group, the cycloalkyl group, the alkoxy group, the aryl group, the heterocyclic group, the aralkyl group, and the aryloxy group include the same groups as described in the foregoing. The alkoxycarbonyl group is represented by —COOY′, and examples of Y′ include the same groups as those of the alkyl group.

M represents aluminum (Al), gallium (Ga), or indium (In), preferably aluminum (Al).

L in the formula (A) represents a group represented by the following formula (A′) or (A″).

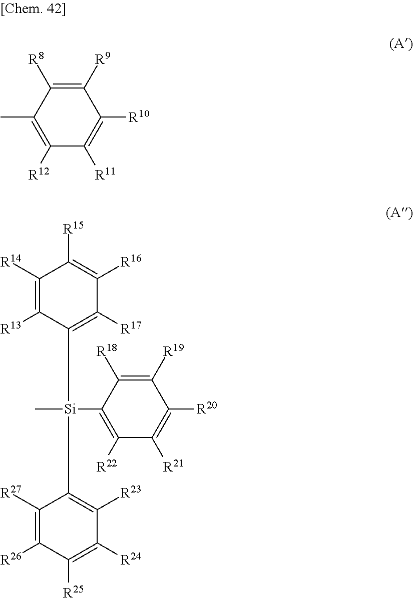

In the formula (A′), R⁸ to R¹² each independently represent a hydrogen atom or a substituted or unsubstituted hydrocarbon group having 1 to 40 carbon atoms, and adjacent groups may form a cyclic structure.

In addition, in the formula (A″), R¹³ to R²⁷ each independently represent a hydrogen atom or a substituted or unsubstituted hydrocarbon group having 1 to 40 carbon atoms, and adjacent groups may form a cyclic structure.

As the hydrocarbon group having 1 to 40 carbon atoms represented by each of R⁸ to R¹² in the general formula (A′) and R¹³ to R²⁷ in the general formula (A″), the same specific examples as those of R² to R⁷ are given.

In addition, examples of the divalent group in R⁸ to R¹² and R¹³ to R²⁷ in the case where adjacent groups form a cyclic structure include a tetramethylene group, a pentamethylene group, a hexamethylene group, a diphenylmethane-2,2′-diyl group, a diphenylethane-3,3′-diyl group, and a diphenylpropane-4,4′-diyl group.

Specific examples of the nitrogen-containing ring metal chelate complex represented by the general formula (A) are shown below. However, the complex is not limited to these exemplary compounds.

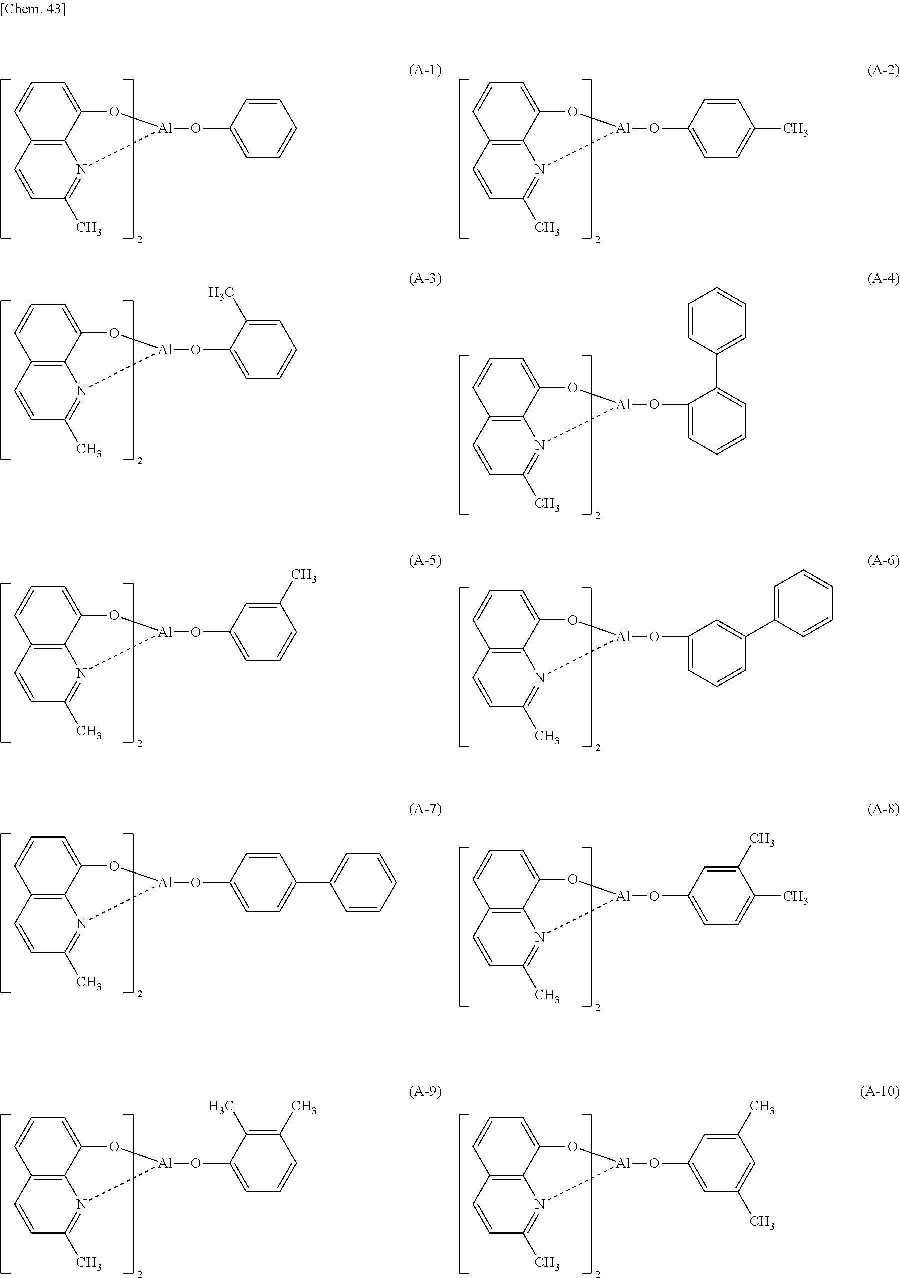

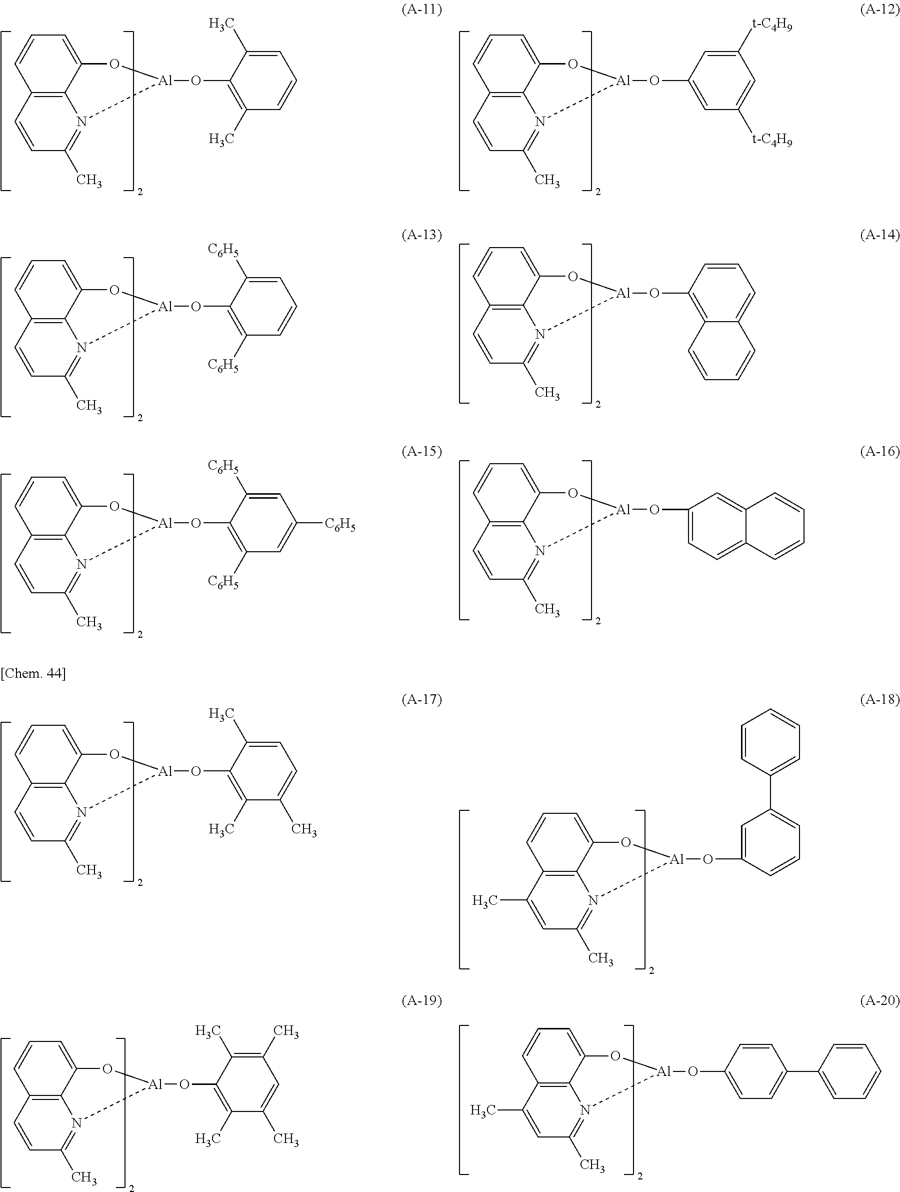

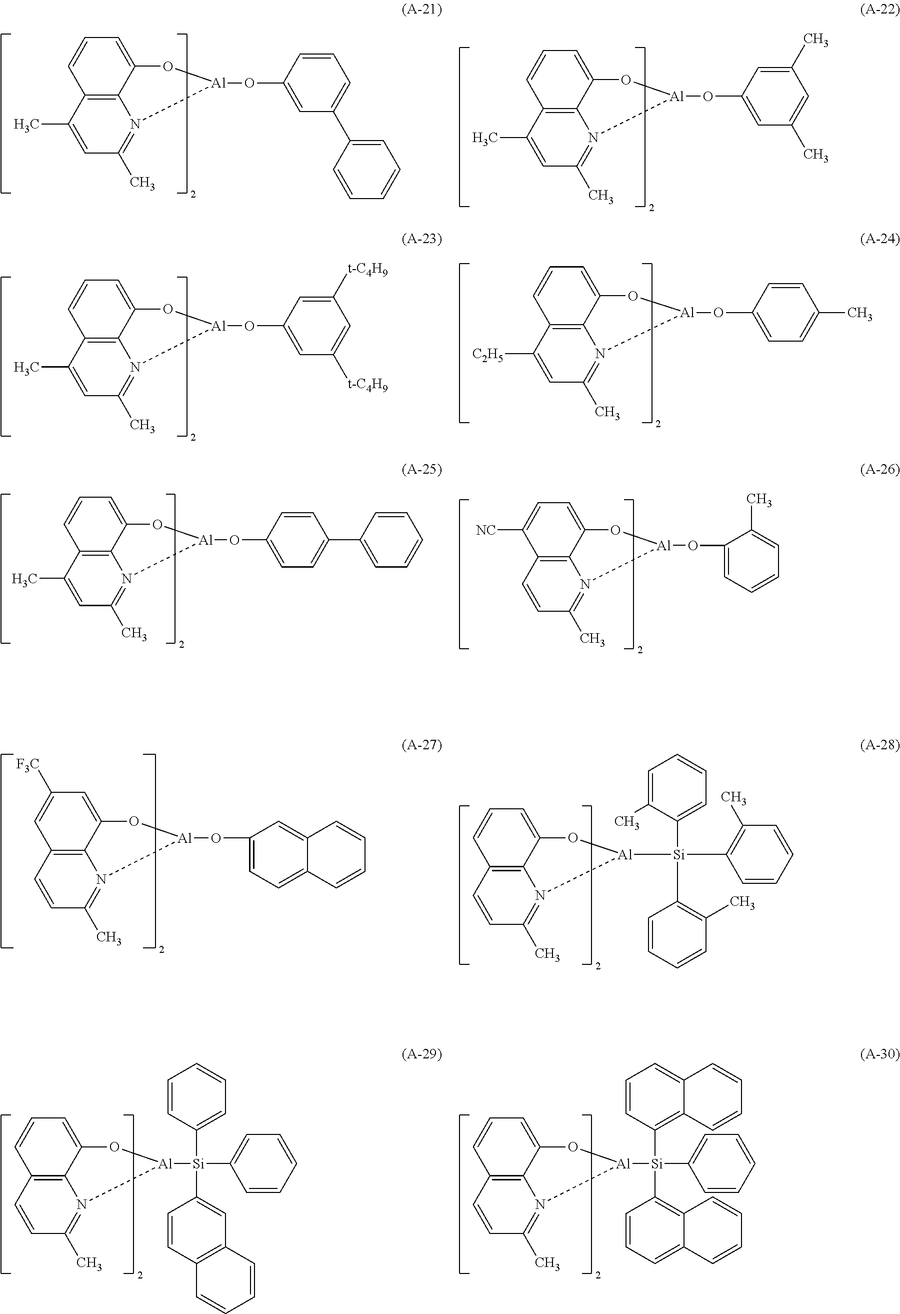

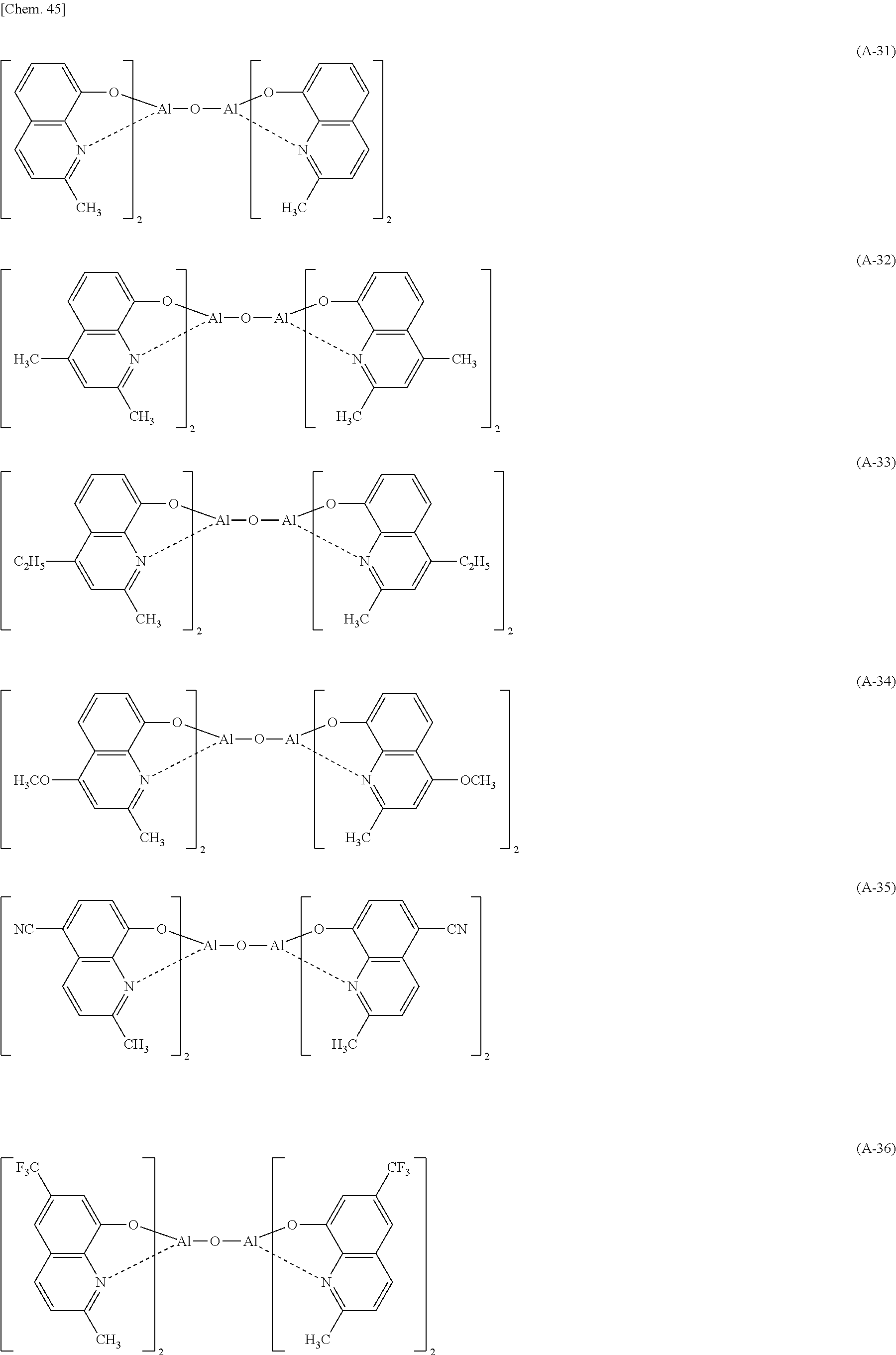

The nitrogen-containing ring derivative as a main component of the electron injecting layer is preferably a nitrogen-containing five-membered ring derivative. Examples of the nitrogen-containing five-membered ring include an imidazole ring, a triazole ring, a tetrazole ring, an oxadiazole ring, a thiadiazole ring, an oxatriazole ring, and a thiatriazole ring. Examples of the nitrogen-containing five-membered ring derivative include a benzoimidazole ring, a benzotriazole ring, a pyridinoimidazole ring, a pyrimidinoimidazole ring, and a pyridazinoimidazole ring. Particularly preferred is the compound represented by the following formula (B).

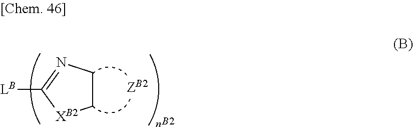

In the formula (B), L^{B} represents a divalent or more linking group. Examples thereof include carbon, silicon, nitrogen, boron, oxygen, sulfur, metals (for example, barium and beryllium), an aryl group, and an aromatic heterocycle. Of those, a carbon atom, a nitrogen atom, a silicon atom, a boron atom, an oxygen atom, a sulfur atom, an aryl group, and an aromatic heterocyclic group are preferred, and a carbon atom, a silicon atom, an aryl group, and an aromatic heterocyclic group are more preferred.

The aryl group and the aromatic heterocyclic group each represented by L^{B} may each have a substituent. The substituent is preferably an alkyl group, an alkenyl group, an alkynyl group, an aryl group, an amino group, an alkoxy group, an aryloxy group, an acyl group, an alkoxycarbonyl group, an aryloxycarbonyl group, an acyloxy group, an acylamino group, an alkoxycarbonylamino group, an aryloxycarbonylamino group, a sulfonylamino group, a sulfamoyl group, a carbamoyl group, an alkylthio group, an arylthio group, a sulfonyl group, a halogen atom, a cyano group, or an aromatic heterocyclic group, more preferably an alkyl group, an aryl group, an alkoxy group, an aryloxy group, a halogen atom, a cyano group, or an aromatic heterocyclic group, still more preferably an alkyl group, an aryl group, an alkoxy group, an aryloxy group, or an aromatic heterocyclic group, particularly preferably an alkyl group, an aryl group, an alkoxy group, or an aromatic heterocyclic group.

Specific examples of L^{B} include the following.

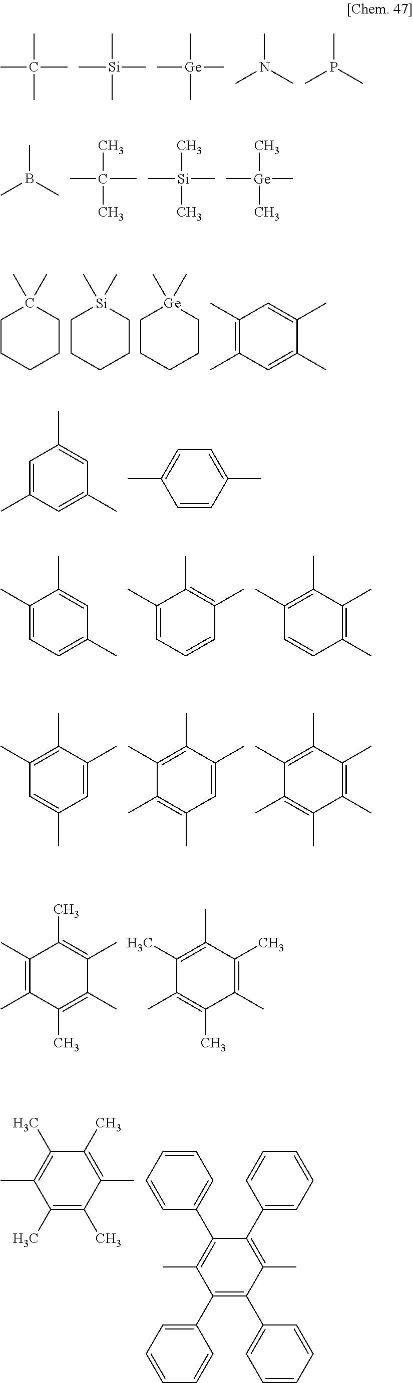

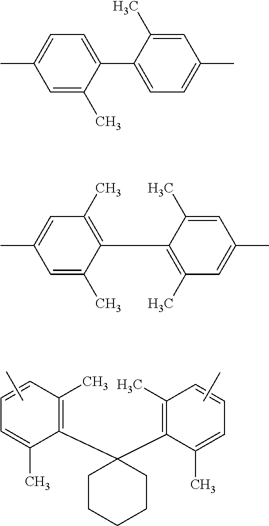

X^{B2} in the formula (B) represents —O—, —S—, or ═N—R^{B2}. R^{B2} represents a hydrogen atom, an aliphatic hydrocarbon group, an aryl group, or a heterocyclic group.

The aliphatic hydrocarbon group represented by R^{B2} is a linear, branched, or cyclic alkyl group (an alkyl group having preferably to 20 carbon atoms, more preferably 1 to 12 carbon atoms, particularly preferably 1 to 8 carbon atoms, such as a methyl group, an ethyl group, an isopropyl group, a t-butyl group, an n-octyl group, an n-decyl group, an n-hexadecyl group, a cyclopropyl group, a cyclopentyl group, or a cyclohexyl group), an alkenyl group (an alkenyl group having preferably 2 to 20 carbon atoms, more preferably 2 to 12 carbon atoms, particularly preferably 2 to 8 carbon atoms, such as a vinyl group, an allyl group, a 2-butenyl group, or a 3-pentenyl group), or an alkynyl group (an alkynyl group having preferably 2 to 20 carbon atoms, more preferably 2 to 12 carbon atoms, particularly preferably 2 to 8 carbon atoms, such as a propargyl group or a 3-pentynyl group). Of those, an alkyl group is preferred.

The aryl group represented by R^{B2} is a group having a single ring or a fused ring. The aryl group has preferably 6 to 30 carbon atoms, more preferably 6 to 20 carbon atoms, still more preferably 6 to 12 carbon atoms, and examples thereof include a phenyl group, a 2-methylphenyl group, a 3-methylphenyl group, a 4-methylphenyl group, a 2-methoxyphenyl group, a 3-trifluoromethylphenyl group, a pentafluorophenyl group, a 1-naphthyl group, and a 2-naphthyl group.

The heterocyclic group represented by R^{B2} is a single ring or a fused ring. The heterocyclic group has preferably 1 to 20 carbon atoms, more preferably 1 to 12 carbon atoms, still more preferably 2 to 10 carbon atoms, and is preferably an aromatic heterocyclic group containing at least one of a nitrogen atom, an oxygen atom, a sulfur atom, and a selenium atom. Examples of the heterocyclic group include pyrrolidine, piperidine, piperazine, morpholine, thiophene, selenophene, furan, pyrrol, imidazole, pyrazole, pyridine, pyrazine, pyridazine, pyrimidine, triazole, triazine, indole, indazole, purine, thiazoline, thiazole, thiadiazole, oxazoline, oxazole, oxadiazole, quinoline, isoquinoline, phthalazine, naphthyridine, quinoxaline, quinazoline, cinnoline, pteridine, acridine, phenanthroline, phenazine, tetrazole, benzoimidazole, benzoxazole, benzothiazole, benzotriazole, tetrazaindene, carbazole, and azepine. Of those, furan, thiophene, pyridine, pyrazine, pyrimidine, pyridazine, triazine, quinoline, phthalazine, naphthyridine, quinoxaline, and quinazoline are preferred, furan, thiophene, pyridine, and quinoline are more preferred, and quinoline is still more preferred.

The aliphatic hydrocarbon group, the aryl group, and the heterocyclic group each of which is represented by R^{B2} may have a substituent, and examples of the substituent include the same substituents as those given for the group represented by L^{B}. In addition, preferred substituents are also the same.

R^{B2} represents preferably an aliphatic hydrocarbon group, an aryl group, or a heterocyclic group, more preferably an aliphatic hydrocarbon group (having preferably 6 to 30 carbon atoms, more preferably 6 to 20 carbon atoms, still more preferably 6 to 12 carbon atoms) or an aryl group, still more preferably an aliphatic hydrocarbon group (having preferably 1 to 20 carbon atoms, more preferably 1 to 12 carbon atoms, still more preferably 2 to 10 carbon atoms).

X^{B2} represents preferably —O— or ═N—R^{B2}, more preferably ═N—R^{B2}, particularly preferably ═N—R^{B2}.

Z^{B2} represents a group of atoms necessary for forming an aromatic ring. The aromatic ring formed with the group of atoms represented by Z^{B2} may be any one of an aromatic hydrocarbon ring and an aromatic heterocycle. Specific examples of the aromatic ring include a benzene ring, a pyridine ring, a pyrazine ring, a pyrimidine ring, a pyridazine ring, a triazine ring, a pyrrole ring, a furan ring, a thiophene ring, a selenophene ring, a tellurophene ring, an imidazole ring, a thiazole ring, a selenazole ring, a tellurazole ring, a thiadiazole ring, an oxadiazole ring, and a pyrazole ring. Of those, a benzene ring, a pyridine ring, a pyrazine ring, a pyrimidine ring, and a pyridazine ring are preferred, and a benzene ring, a pyridine ring, and a pyrazine ring are more preferred. In addition, a benzene ring and a pyridine ring are still more preferred, and a pyridine ring is particularly preferred.

The aromatic ring formed with the group of atoms represented by Z^{B2} may form a fused ring with another ring, and may have a substituent. Examples of the substituent include the same substituents as those given for the group represented by L^{B}. Of those, preferred are an alkyl group, an alkenyl group, an alkynyl group, an aryl group, an amino group, an alkoxy group, an aryloxy group, an acyl group, an alkoxycarbonyl group, an aryloxycarbonyl group, an acyloxyl group, an acylamino group, an alkoxycarbonylamino group, an aryloxycarbonylamino group, a sulfonylamino group, a sulfamoyl group, a carbamoyl group, an alkylthio group, an arylthio group, a sulfonyl group, a halogen atom, a cyano group, and a heterocyclic group. More preferred are an alkyl group, an aryl group, an alkoxy group, an aryloxy group, a halogen atom, a cyano group, and a heterocyclic group. Still more preferred are an alkyl group, an aryl group, an alkoxyl group, an aryloxy group, and an aromatic heterocyclic group, and particularly preferred are an alkyl group, an aryl group, an alkoxyl group, and an aromatic heterocyclic group.

n^{B2} represents an integer of 1 to 4, preferably 2 to 3.

Of the nitrogen-containing five-membered ring derivatives each represented by the formula (B), a compound represented by the following formula (B′) is more preferred.

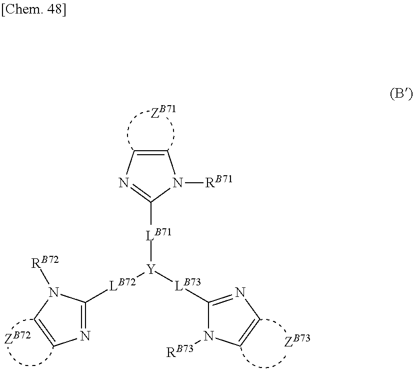

In the formula (B′), R^{B71}, R^{B72}, and R^{B73} each represent the same atom or group as those represented by R^{B2} in the formula (B). The range of preferred examples are also the same.

Z^{B71}, Z^{B72}, and Z^{B73} each represent the same groups as those in the case of Z^{B2} in the formula (B). The range of preferred examples also the same.

L^{B71}, L^{B72}, and L^{B73} each represent a linking group, and examples thereof include the linking groups described as the examples of the divalent linking group represented by L^{B} in the formula (B). The linking group is preferably a single bond, a divalent aromatic hydrocarbon ring group, a divalent aromatic heterocyclic group, or a combination of those groups, more preferably a single bond. The linking group represented by L^{B71}, L^{B72}, and L^{B73} may have a substituent. Examples of the substituent include the same substituents as those given for the group represented by L^{B} in the formula (B), and preferred substituents are also the same.

Y represents a nitrogen atom, a 1,3,5-benzenetriyl group, or a 2,4,6-triazinetriyl group. The 1,3,5-benzenetriyl group may have a substituent at 2,4,6-positions. Examples of the substituent include an alkyl group, an aromatic hydrocarbon ring group, and a halogen atom.

Specific examples of the nitrogen-containing five-membered ring derivative represented by the formula (B) or the formula (B′) are shown in the following, but the derivative is not limited to the exemplary compounds.

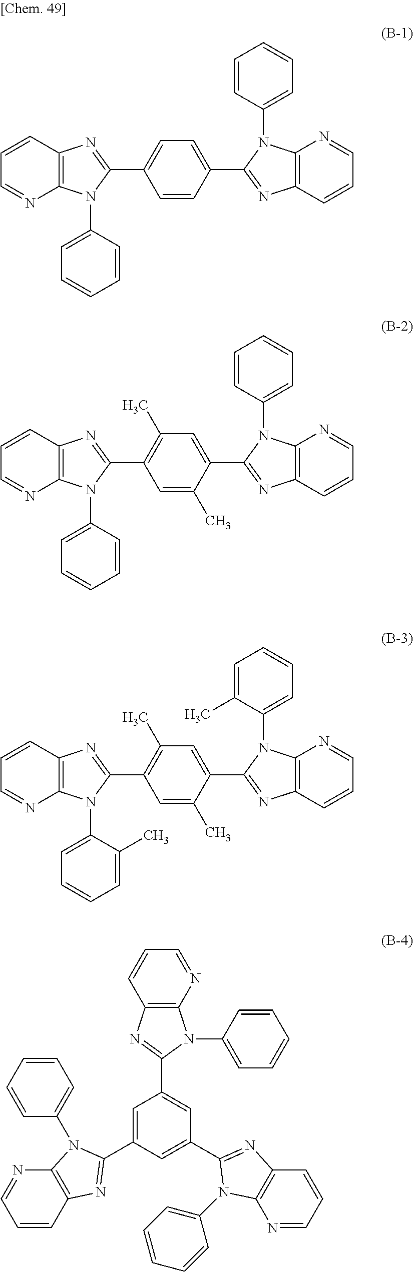

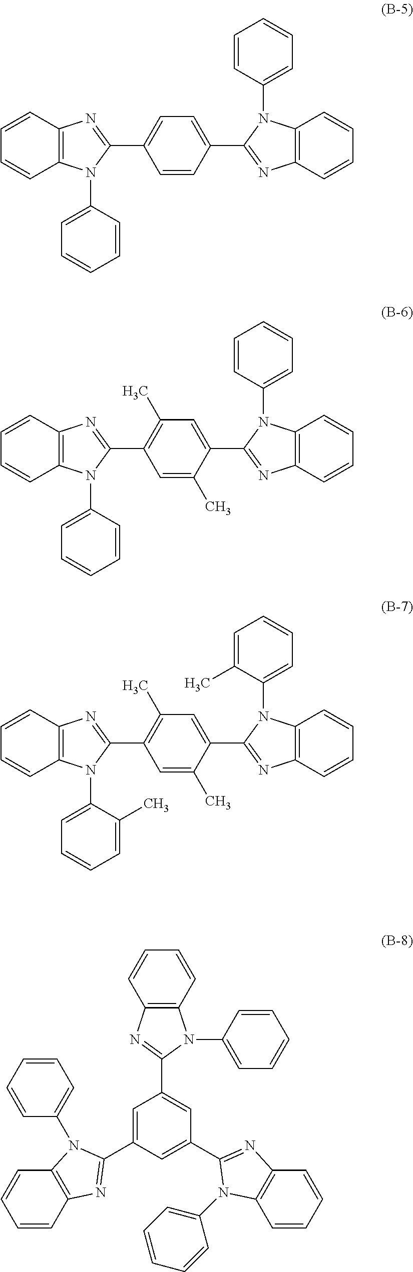

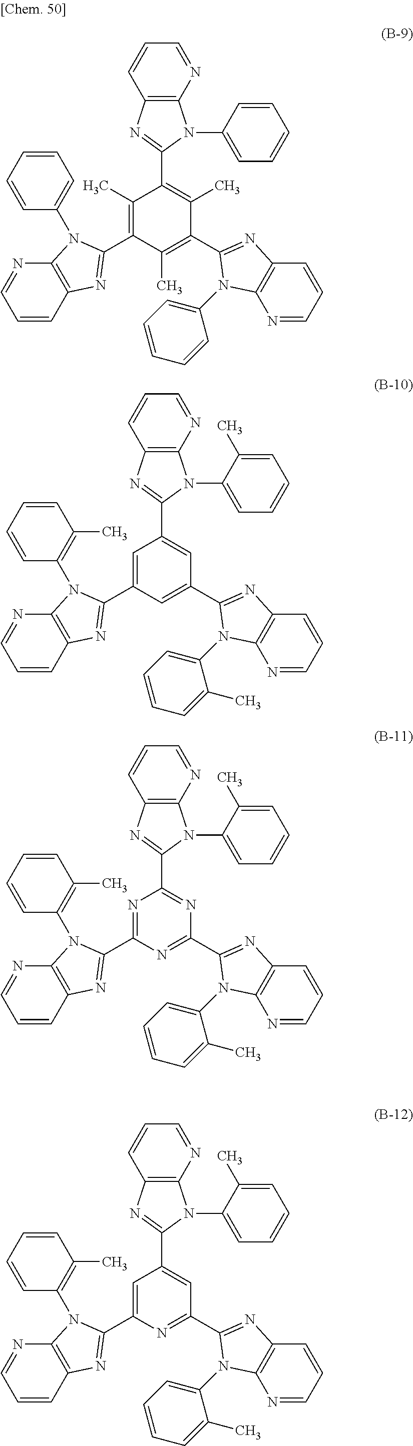

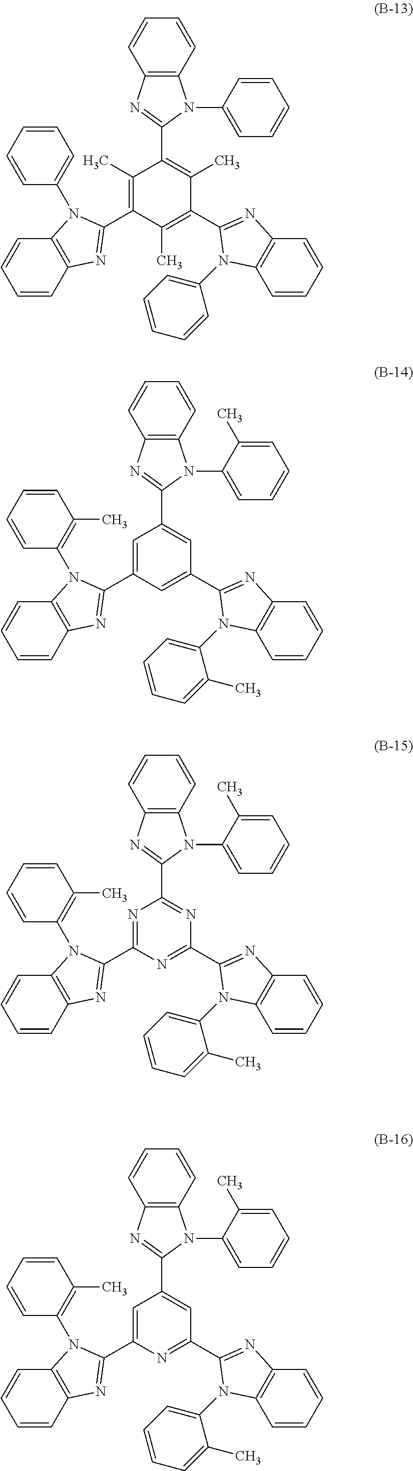

As a compound for constructing each of the electron injecting layer and the electron transporting layer, there is also given, for example, a compound having a structure obtained by combining an electron-deficient, nitrogen-containing five-membered ring skeleton or electron-deficient, nitrogen-containing six-membered ring skeleton and a substituted or unsubstituted indole skeleton, substituted or unsubstituted carbazole skeleton, or substituted or unsubstituted azacarbazole skeleton. In addition, a suitable electron-deficient, nitrogen-containing five-membered ring skeleton or electron-deficient, nitrogen-containing six-membered ring skeleton is, for example, a molecular skeleton such as a pyridine, pyrimidine, pyrazine, triazine, triazole, oxadiazole, pyrazole, imidazole, quinoxaline, or pyrrole skeleton, or benzimidazole or imidazopyridine obtained when two or more of them fuse with each other. Of those combinations, a preferred combination is, for example, a combination of a pyridine, pyrimidine, pyrazine, or triazine skeleton and a carbazole, indole; azacarbazole, or quinoxaline skeleton. The skeleton may be substituted or unsubstituted.

Shown below are specific examples of electron transporting compounds.

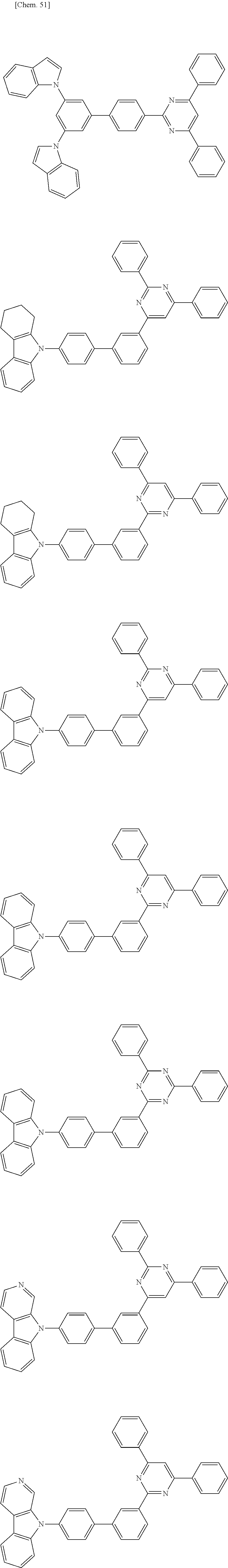

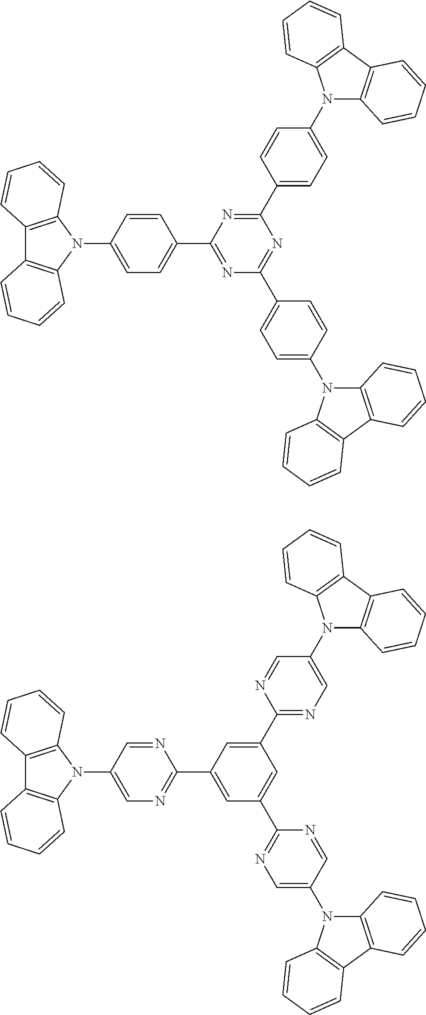

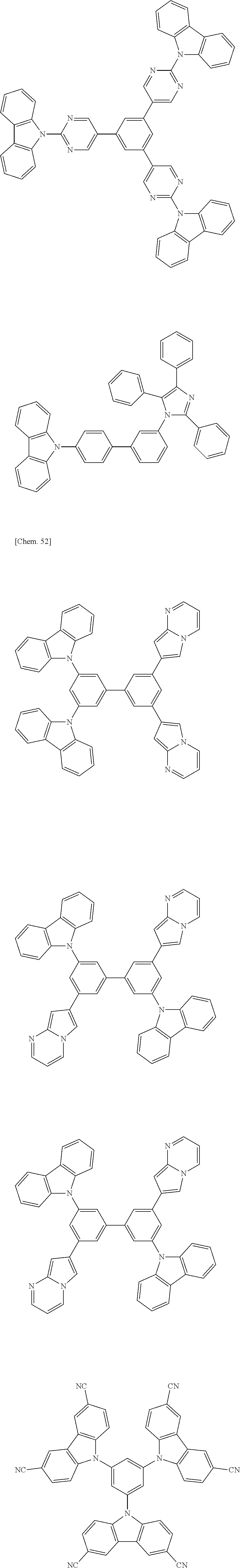

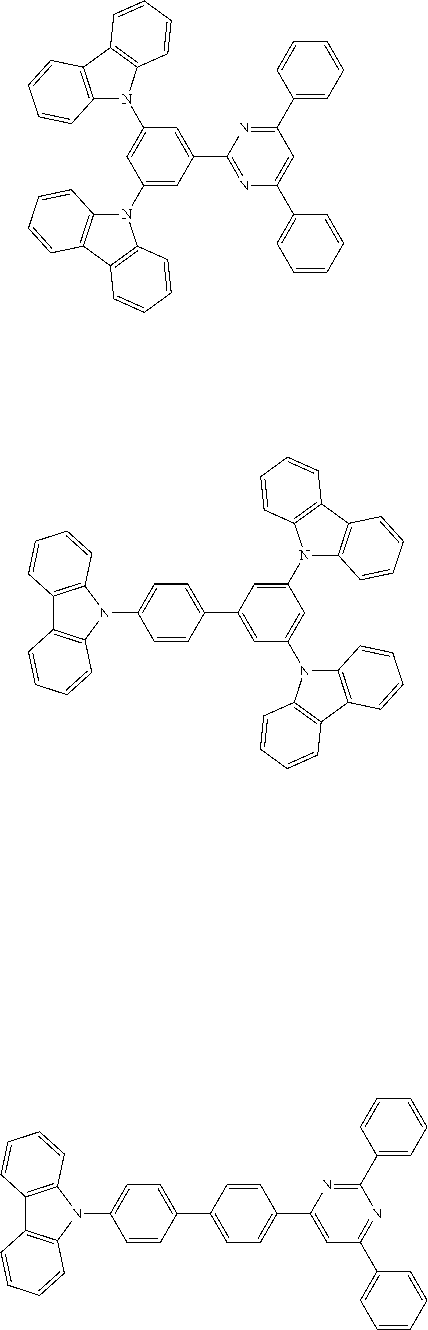

In particular, in the organic EL device of the present invention, a benzimidazole derivative represented by any one of the following formulae (21) to (23) is preferred as the nitrogen-containing five-membered derivative.

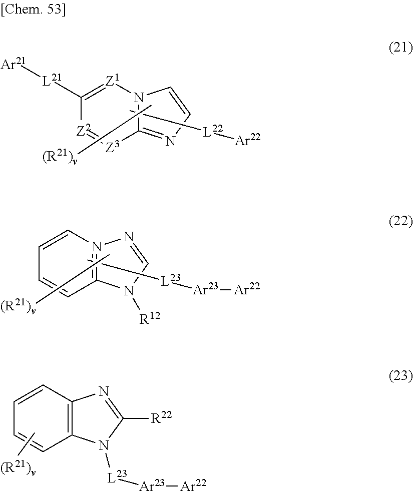

(In the formulae (21) to (23):

Z¹, Z², and Z³ each independently represent a nitrogen atom or a carbon atom;

R²¹ and R²² each independently represent a substituted or unsubstituted aryl group having 6 to 50 carbon atoms, a substituted or unsubstituted heteroaryl group having 3 to 50 carbon atoms, an alkyl group having 1 to 20 carbon atoms, a halogen atom-substituted alkyl group having 1 to 20 carbon atoms, or an alkoxy group having 1 to 20 carbon atoms;

v represents an integer of 0 to 5, and when v represents an integer of 2 or more, a plurality of R²¹'s may be identical to or different from each other, and a plurality of adjacent R²¹'s may be bonded to each other to form a substituted or unsubstituted aromatic hydrocarbon ring;

Ar²¹ represents a substituted or unsubstituted aryl group having 6 to 50 carbon atoms, or a substituted or unsubstituted heteroaryl group having 3 to 50 carbon atoms;

Ar²² represents a hydrogen atom, an alkyl group having 1 to 20 carbon atoms, a halogen atom-substituted alkyl group having 1 to 20 carbon atoms, an alkoxy group having 1 to 20 carbon atoms, a substituted or unsubstituted aryl group having 6 to 50 carbon atoms, or a substituted or unsubstituted heteroaryl group having 3 to 50 carbon atoms,

provided that one of Ar²¹ and Ar²² represents a substituted or unsubstituted fused ring group having 10 to 50 carbon atoms, or a substituted or unsubstituted heterocyclic fused ring group having 9 to 50 ring atoms;

Ar²³ represents a substituted or unsubstituted arylene group having 6 to 50 carbon atoms, or a substituted or unsubstituted heteroarylene group having 3 to 50 carbon atoms; and

L²¹, L²², and L²³ each independently represent a single bond, a substituted or unsubstituted arylene group having 6 to 50 carbon atoms, a substituted or unsubstituted heterocyclic fused ring group having 9 to 50 ring atoms, or a substituted or unsubstituted fluorenylene group.)

Specific examples of the nitrogen-containing five-membered ring derivatives represented by the formulae (21) to (23) are given below. However, the present invention is not limited to these exemplified compounds.

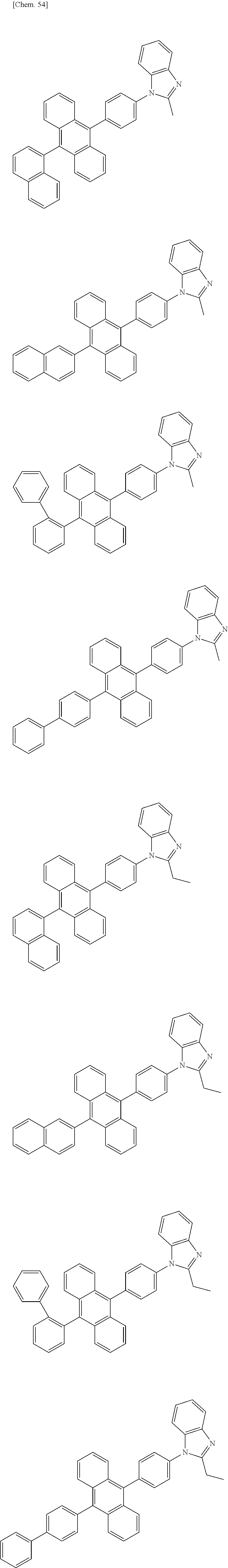

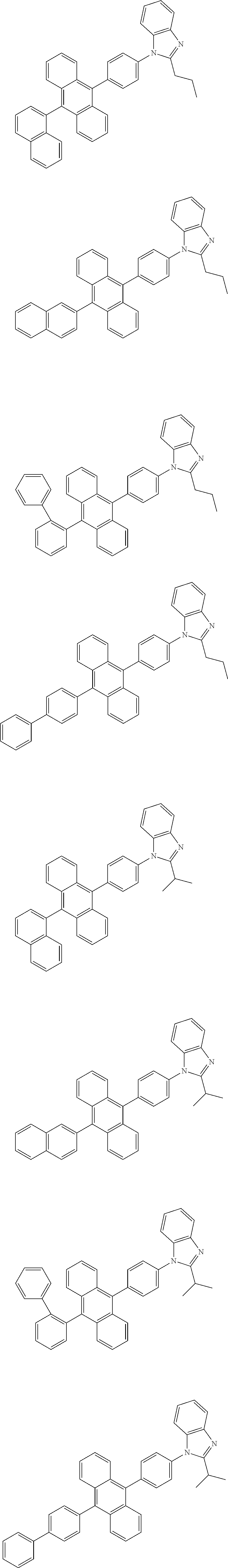

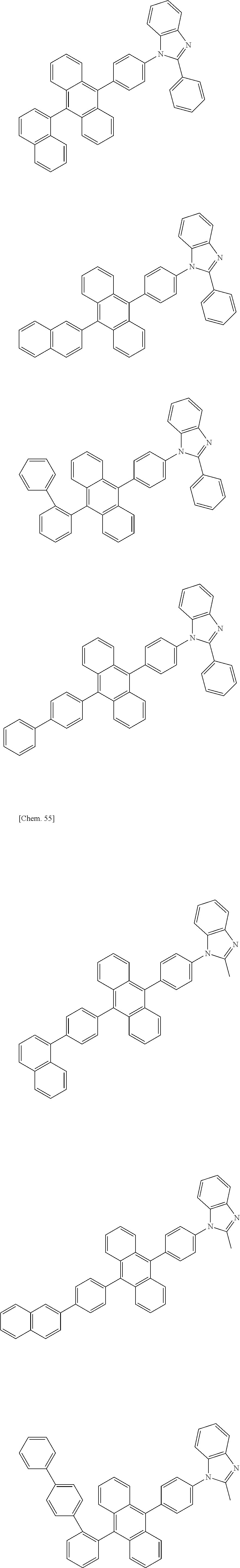

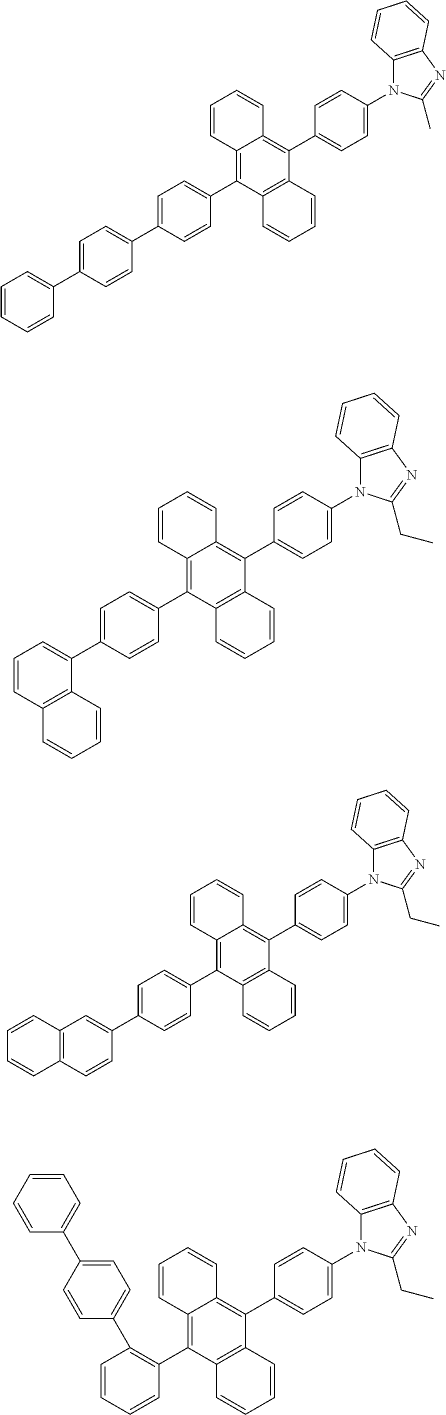

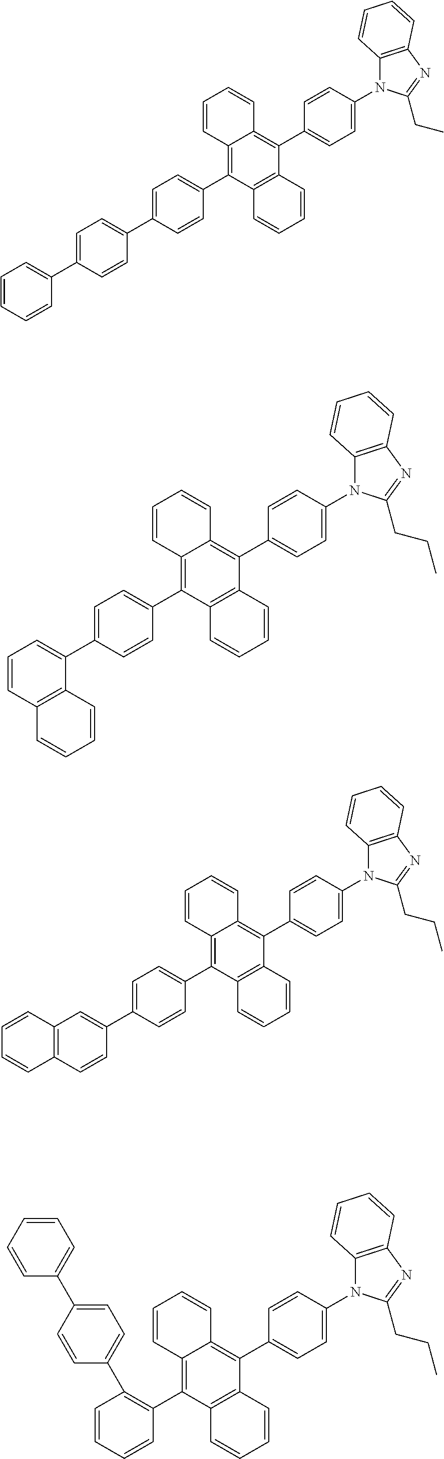

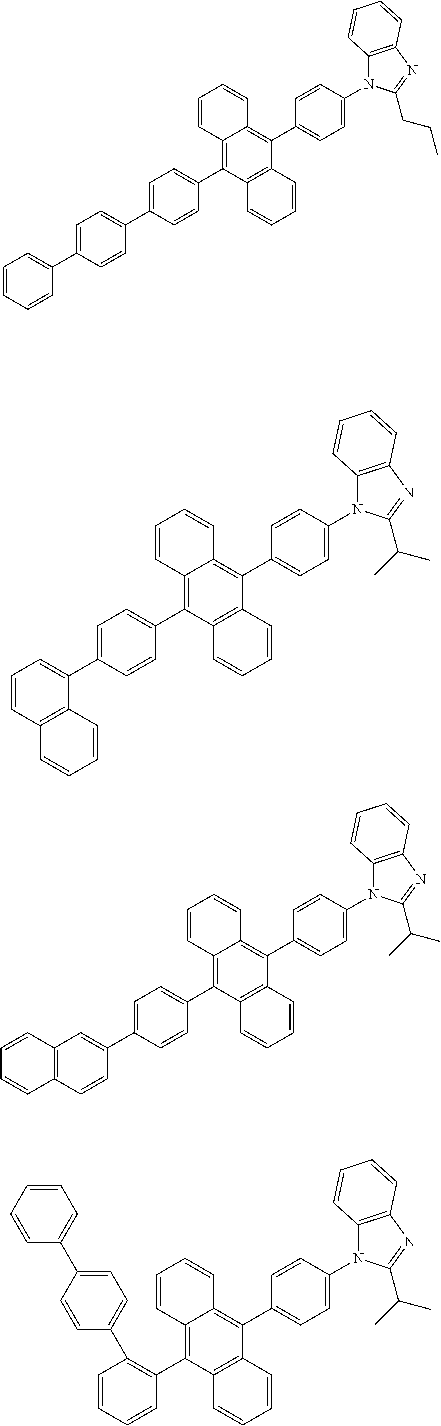

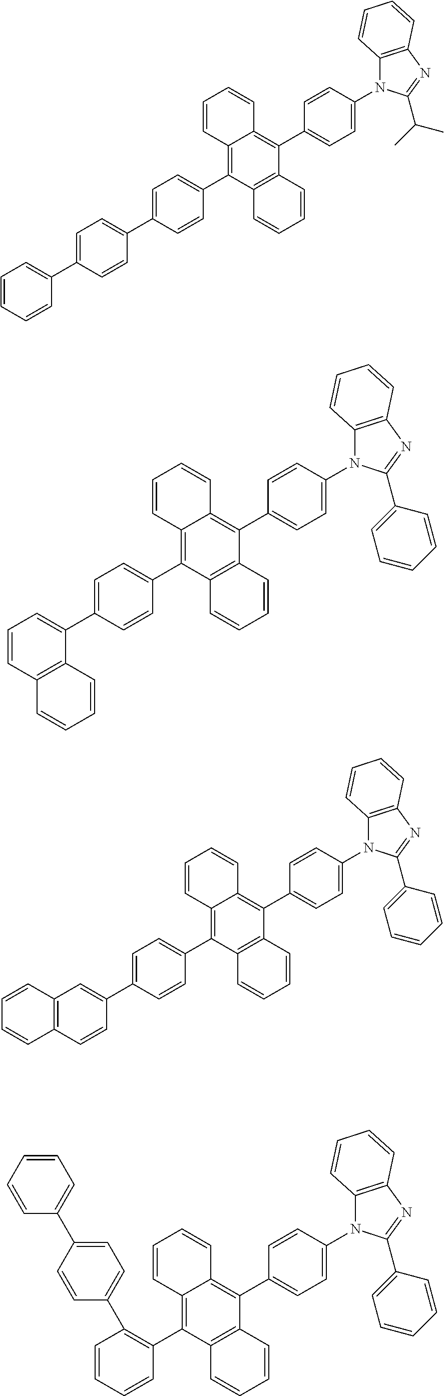

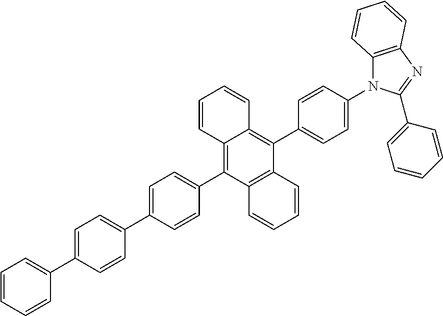

Each of the electron injecting layer and the electron transporting layer may be of a monolayer structure formed of one or two or more kinds of the materials, or may be of a multi-layered structure formed of the plurality of layers identical to or different from each other in composition. Those are each preferably a π-electron-deficient, nitrogen-containing heterocyclic group.

In addition, an insulator or semiconductor serving as an inorganic compound as well as the nitrogen-containing ring derivative is preferably used as a component of the electron injecting layer. When the electron injecting layer is formed of an insulator or semiconductor, current leakage can be effectively prevented, and the electron injecting property of the layer can be improved.

As the insulator, at least one metal compound selected from the group consisting of alkali metal chalcogenides, alkaline earth metal chalcogenides, alkali metal halides, and alkaline earth metal halides is preferably used. It is preferred that the electron injecting layer be formed of the alkali metal chalcogenide or the like because the electron injecting property can be further improved. Specifically, preferred examples of the alkali metal chalcogenide include Li₂O, K₂O, Na₂S, Na₂Se, and Na₂O, and preferred examples of the alkaline earth metal chalcogenide include CaO, BaO, SrO, BeO, BaS, and CaSe. In addition, preferred examples of the alkali metal halide include LiF, NaF, KF, LiCl, KCl, and NaCl. Further, preferred examples of the alkaline earth metal halide include fluorides such as CaF₂, BaF₂, SrF₂, MgF₂, and BeF₂ and halides other than the fluorides.

In addition, examples of the semiconductor include only one kind of oxides, nitrides, and oxynitrides containing at least one element selected from the group consisting of Ba, Ca, Sr, Yb, Al, Ga, In, Li, Na, Cd, Mg, Si, Ta, Sb, and Zn, and a combination of two or more kinds thereof. In addition, it is preferred that the inorganic compound forming the electron injecting layer form a microcrystalline or amorphous insulating thin film. When the electron injecting layer is formed of the insulating thin film, a more uniform thin film can be formed, and defects of pixels such as dark spots can be decreased. It should be noted that examples of the inorganic compound include alkali metal chalcogenides, alkaline earth metal chalcogenides, alkali metal halides, and alkaline earth metal halides described above.

In addition, the above-mentioned reduction-causing dopant can be preferably incorporated into the electron injecting layer in the present invention.

In the present invention, the light emitting material is preferably a metal complex whose light emission shows a local maximum at a wavelength of 500 nm or less.

A light emitting material having a short luminous wavelength generally has a large excited triplet energy gap.

Here, when a hole transporting layer is formed by using α-NPD like the organic EL device described in US 2006-0088728 A1, the triplet energy gap of the hole transporting layer is smaller than the excited triplet energy gap of the light emitting material in some cases because α-NPD has an excited triplet energy gap of 2.5 eV or less.

In such case, luminous efficiency may reduce because the excited triplet energy of a light emitting layer leaks to the adjacent hole transporting layer and hence deactivates without contributing to the light emission.

In contrast, in the present invention, high luminous efficiency can be maintained even when a light emitting material having a short luminous wavelength is adopted because the first hole transporting layer and the second hole transporting layer are formed by using the compounds represented by the formulae (1) to (5) each having a larger excited triplet energy gap than that of α-NPD.

(32) An electron acceptable substance is joined to the hole transporting layer.

With such construction, low-voltage driving and high-efficiency light emission are realized by effects described in patents to be described later.

An inorganic compound such as p-type Si or p-type SiC, an electron acceptable inorganic oxide such as molybdenum oxide, an electron acceptable organic compound such as a TCNQ derivative, or the like as well as a hexaazatriphenylene derivative or the like described in JP 3614405 B2 or JP 3571977 B2, or U.S. Pat. No. 4,780,536 A can be suitably used as the electron acceptable substance to be added or joined to the first hole transporting layer or second hole transporting layer of the present invention.

The hole transporting layer of the present invention preferably has a layer containing an electron acceptable compound on the side of the first hole transporting layer closer to the anode.

A compound represented by the following general formula (10) or (11) is preferably used as the electron acceptable compound.

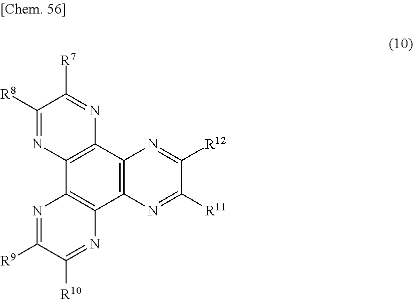

[In the above-mentioned general formula (10), R⁷ to R¹² each independently represent a cyano group, —CONH₂, a carboxyl group, or —COOR¹³ (R¹³ represents an alkyl group having 1 to 20 carbon atoms), or R⁷ and R⁸, R⁹ and R¹⁰, or R¹¹ and R¹² are bonded to each other to represent a group represented by —CO—O—CO—.]

Examples of the above-mentioned alkyl group include linear, branched, and cyclic alkyl groups. The group has preferably 1 to 12 carbon atoms, more preferably 1 to 8 carbon atoms, and specific examples of such group include a methyl group, an ethyl group, an n-propyl group, an isopropyl group, an n-butyl group, a sec-butyl group, a t-butyl group, an n-hexyl group, an n-octyl group, an n-decyl group, an n-hexadecyl group, a cyclopropyl group, a cyclopentyl group, and a cyclohexyl group.

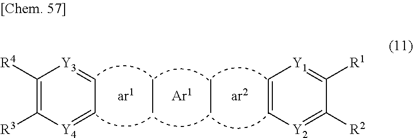

[In the above-mentioned general formula (11), Ar¹ represents a fused ring having 6 to 24 ring carbon atoms or a heterocyclic ring having 6 to 24 ring atoms, and ar¹ and ar² may be identical to or different from each other, and each represent the following formula (i) or (ii).

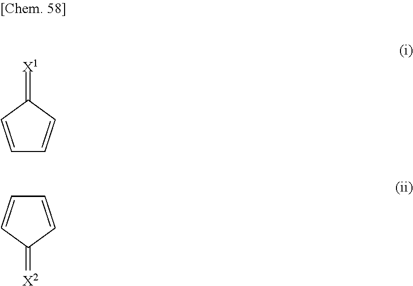

{In the formulae, X¹ and X² may be identical to or different from each other, and each represent any one of the divalent groups represented by the following formulae (a) to (g).

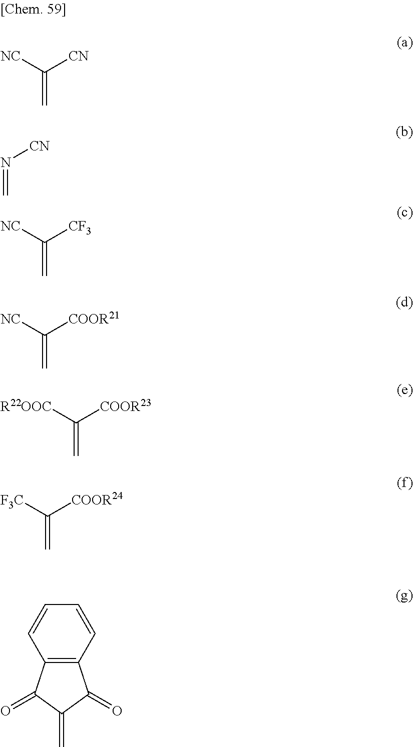

(In the formulae, R²¹ to R²⁴ may be identical to or different from one another, and each represent a hydrogen atom, a substituted or unsubstituted fluoroalkyl group having 1 to 20 carbon atoms, a substituted or unsubstituted alkyl group having 1 to 20 carbon atoms, a substituted or unsubstituted aryl group having 6 to 50 carbon atoms, or a substituted or unsubstituted heterocyclic group having 3 to 50 ring atoms, and R²² and R²³ may be bonded to each other to form a ring.)}

R¹ to R⁴ in the general formula (11) may be identical to or different from one another, and each represent a hydrogen atom, a substituted or unsubstituted alkyl group having 1 to 20 carbon atoms, a substituted or unsubstituted aryl group having 6 to 50 carbon atoms, a substituted or unsubstituted heterocyclic group having 3 to 50 ring atoms, a halogen atom, a substituted or unsubstituted fluoroalkyl group having 1 to 20 carbon atoms, a substituted or unsubstituted alkoxy group having 1 to 20 carbon atoms, a substituted or unsubstituted fluoroalkoxy group having 1 to 20 carbon atoms, a substituted or unsubstituted aryloxy group having 6 to 50 carbon atoms, or a cyano group, and groups adjacent to each other out of R¹ to R⁴ may be bonded to each other to form a ring, Y¹ to Y⁴ may be identical to or different from one another, and each represent —N═, —CH═, or C(R⁵)═, and R⁵ represents a substituted or unsubstituted alkyl group having 1 to 20 carbon atoms, a substituted or unsubstituted aryl group having 6 to 50 carbon atoms, a substituted or unsubstituted heterocyclic group having 3 to 50 ring atoms, a halogen atom, a substituted or unsubstituted fluoroalkyl group having 1 to 20 carbon atoms, a substituted or unsubstituted alkoxy group having 1 to 20 carbon atoms, a substituted or unsubstituted fluoroalkoxy group having 1 to 20 carbon atoms, a substituted or unsubstituted aryloxy group having 6 to 50 carbon atoms, or a cyano group.]

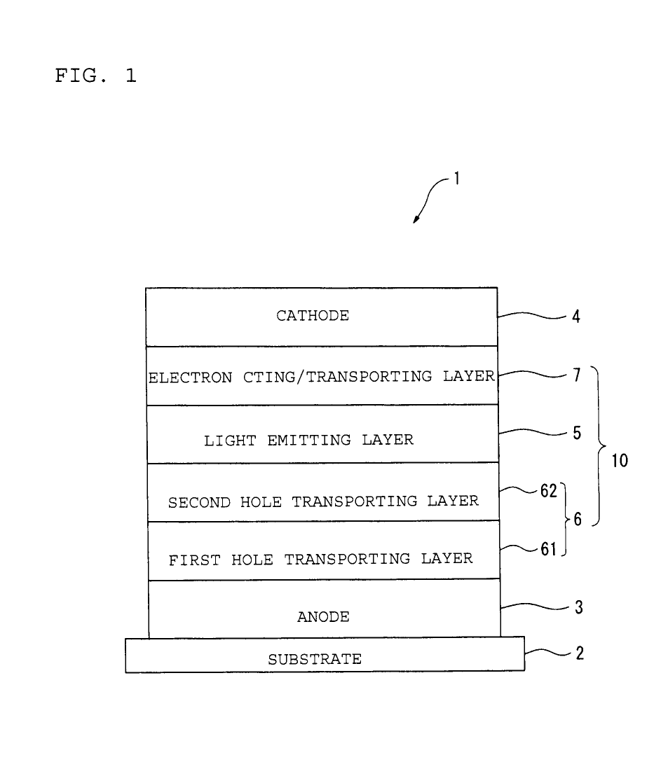

FIG. 1 illustrates the schematic construction of an embodiment of the organic EL device of the present invention.

An organic EL device 1 has a transparent substrate 2, an anode 3, a cathode 4, and a light emitting layer 5 placed between the anode 3 and the cathode 4. Provided between the light emitting layer and the anode 3 is a hole transporting layer 6 having a first hole transporting layer 61 and a second hole transporting layer 62 in the stated order from the side of the anode 3. An electron injecting/transporting layer 7 is provided between the light emitting layer 5 and the cathode 4.

The first hole transporting layer 61 contains the compound represented by the general formula (1) and the second hole transporting layer 62 contains the compound represented by the general formula (2).

Here, each of the compounds represented by the general formulae (1) and (2) in the first hole transporting layer 61 and the second hole transporting layer 62 is not limited to one kind. In other words, the first hole transporting layer 61 may contain a plurality of compounds each represented by the general formula (1), and the second hole transporting layer 62 may contain a plurality of compounds each represented by the general formula (2).

The content of the compound represented by the general formula (1) in the first hole transporting layer is preferably 90 mass % or more. In addition, the content of the compound represented by the general formula (2) in the second hole transporting layer is preferably 90 mass % or more.

In the present invention, the anode in the organic EL device has the function of injecting holes into the hole injecting layer or the hole transporting layer. It is effective that the anode has a work function of 4.5 eV or more. Specific examples of the material for the anode used in the present invention include indium tin oxide alloys (ITO), tin oxide (NESA), gold, silver, platinum, and copper. In addition, as the cathode, a material having a small work function is preferred for the purpose of injecting electrons into an electron injecting layer or a light emitting layer. The material for the cathode is not particularly limited, and specifically, indium, aluminum, magnesium, a magnesium-indium alloy, a magnesium-aluminum alloy, an aluminum-lithium alloy, an aluminum-scandium-lithium alloy, and a magnesium-silver alloy may be used.

The method of forming each layer in the organic EL device of the present invention is not particularly limited.

For example, each layer can be formed in accordance with a conventionally known vacuum vapor deposition process or molecular beam epitaxy process (MBE process), or using a solution prepared by dissolving the compounds into a solvent, in accordance with a dipping process, a spin coating process, a casting process, a bar coating process, a roll coating processor or any other coating process.

The thickness of each layer in the organic EL device of the present invention is not particularly limited. In general, however, an excessively thin layer tends to have defects such as pin holes, whereas an excessively thick layer requires a high applied voltage to decrease the efficiency. Therefore, a thickness in the range of several nanometers to 1 μm is typically preferred.

It should be noted that the construction of the organic EL device of the present invention is not limited to that illustrated in FIG. 1.

For example, a hole injecting layer may be provided between the first hole transporting layer and the anode 3.

In addition, the hole transporting layer 6 is of a two-layered structure formed of the first hole transporting layer 61 and the second hole transporting layer 62.

Further, a hole blocking layer may be provided between the light emitting layer 5 and the electron injecting/transporting layer 7.

With the hole blocking layer, a hole can be trapped in the light emitting layer 5 so that the probability of charge recombination in the light emitting layer 5 may be increased. As a result, the luminous efficiency can be improved.

An organic EL device of a second invention of the present application is an organic electroluminescence device, including an anode, a cathode, and an organic thin-film layer provided between the anode and the cathode, in which:

the organic thin-film layer has a light emitting layer containing a host material and a light emitting material, and a hole transporting layer provided on a side closer to the anode than the light emitting layer;

the hole transporting layer has a layer containing an electron acceptable compound and a first hole transporting layer in the stated order from the anode;

the electron acceptable compound is represented by the above-mentioned general formula (10); and

the first hole transporting layer contains a compound represented by the above-mentioned general formula (2).

Any other construction of the organic EL device of the second invention of the present application is the same as that of the organic EL device of the first invention of the present application.

### EXAMPLES

Hereinafter, the present invention is more specifically described by way of examples. However, the present invention is by no means limited thereto.

### Example 1-1

A glass substrate provided with an ITO transparent electrode measuring 25 mm by 75 mm by 1.1 mm (thickness) (manufactured by ASAHI GLASS CO., LTD.) was subjected to ultrasonic cleaning in isopropyl alcohol for 5 minutes. Then, the substrate was subjected to UV-ozone cleaning for 30 minutes.

The glass substrate provided with a transparent electrode line after the cleaning was mounted on a substrate holder of a vacuum deposition apparatus. First, Compound X1 shown below was formed into a film having a thickness of 40 nm by resistance heating so as to serve as a first hole transporting layer and cover the transparent electrode on the surface of the glass substrate on the side where the transparent electrode line was formed.

Subsequent to the film formation of the first hole transporting layer, Compound Y1-1 (Af: 2.48 eV, Eg(S): 3.12 eV, Ip: 5.60 eV) shown below was formed into a film having a thickness of 20 nm on the film by resistance heating so as to serve as a second hole transporting layer.

Further, Compound H1 as a host material and Compound D1 as a phosphorescent light emitting material were co-deposited from the vapor onto the second hole transporting layer by resistance heating so as to have a thickness of 40 nm. The concentration of Compound D1 was 7.5%. The co-deposited film functions as a light emitting layer.

Further, Compound HB was co-deposited from the vapor onto the light emitting layer by resistance heating so as to have a thickness of 10 nm. The film functions as a hole blocking layer.

Then, subsequent to the film formation of the hole blocking layer, Compound ET1 was formed into a film having a thickness of 30 nm. The ET1 film functions as an electron transporting layer.

Next, LiF was formed into a film having a thickness of 1 nm at a film formation rate of 0.1 Å/min so as to serve as an electron injectable electrode (cathode). Metal Al was deposited from the vapor onto the LiF film so that a metal cathode having a thickness of 80 nm was formed. Thus, an organic EL device was produced.

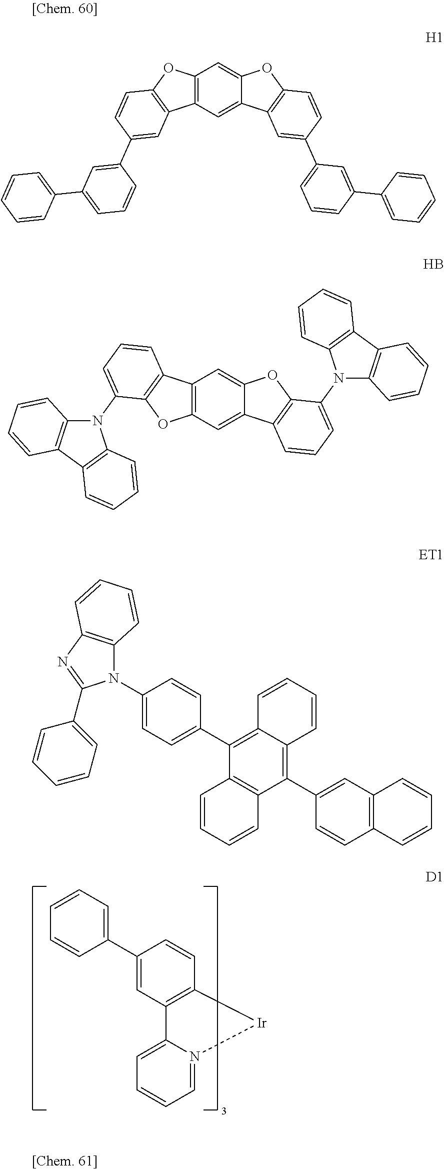

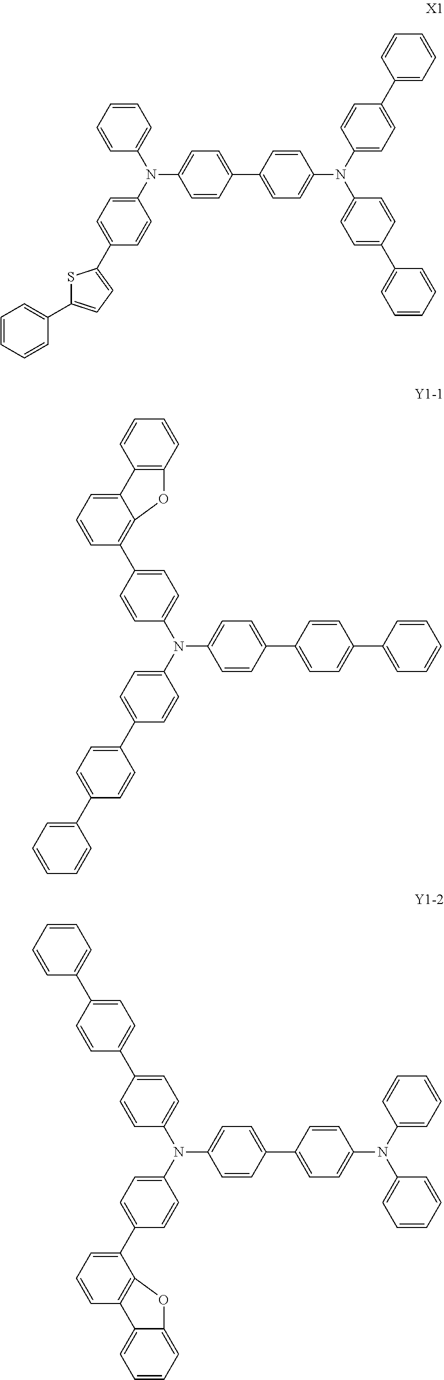

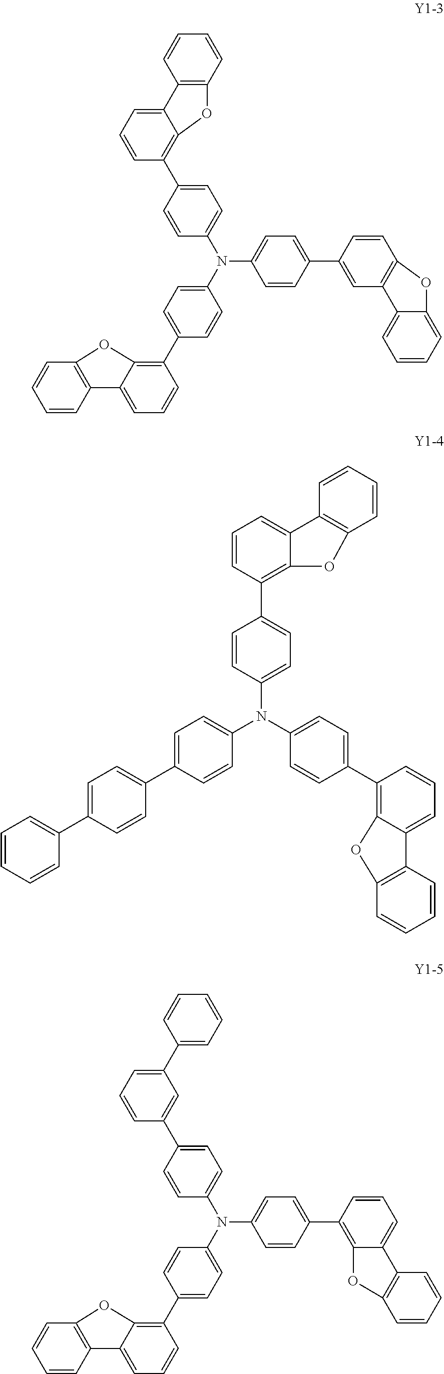

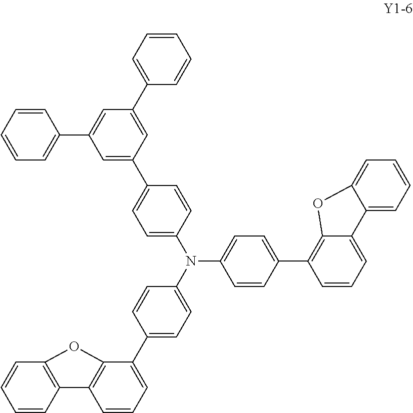

In Example 1-1, the Af, the Eg(S), and the Ip were each measured as described below.

### Af (Affinity Level):

The affinity level was specified by the ionization potential Ip and the optical energy gap Eg(S) as described below.

Af=Ip−Eg(S)

### Eg(S) (Optical Energy Gap):

The optical energy gap was determined by converting a wavelength value for a point of intersection of the tangent of the absorption spectrum of a toluene dilute solution of the material at longer wavelengths and a baseline (zero absorption) into energy.

### Ip (Ionization Potential):

The ionization potential is energy needed for removing an electron from the compound to ionize the compound, and is a value measured with an ultraviolet photoelectron spectrometer (AC-3, Riken Keiki Co., Ltd.).

### Example 1-2

An organic EL device was produced in the same manner as in Example 1-1 except that Compound Y1-2 was used as the second hole transporting layer in Example 1-1.

### Example 1-3

An organic EL device was produced in the same manner as in Example 1-1 except that Compound Y1-3 (Af: 2.45 eV, Eg(S): 3.17 eV, Ip: 5.62 eV, Eg(T): 2.63 eV) was used as the second hole transporting layer in Example 1-1.

It should be noted that the Af, the Eg(S), and the Ip were measured in the same manner as in Example 1-1, and the Eg(T) was measured as described below.

### Eg(T) (Triplet Energy Gap):

The measurement was performed on the basis of a phosphorescence spectrum.

The material was dissolved in an EPA solvent (containing diethyl ether, isopentane, and ethanol at a volume ratio of 5:5:2) at 10 μmol/L so that a sample for phosphorescence measurement was prepared. The sample for phosphorescence measurement was charged into a quartz cell, cooled to 77 K, and irradiated with excitation light. Then, the wavelength of radiated phosphorescence was measured.

A tangent was drawn to the rise-up of the resultant phosphorescence spectrum at shorter wavelengths, and then a wavelength value for a point of intersection of the tangent and a baseline was converted into energy. The resultant value was defined as the excited triplet energy gap Eg(T).

It should be noted that a commercially available measuring apparatus F-4500 (manufactured by Hitachi, Ltd.) was used in the measurement.

### Example 1-4

An organic EL device was produced in the same manner as in Example 1-1 except that Compound Y1-4 was used as the second hole transporting layer in Example 1-1.

### Example 1-5

An organic EL device was produced in the same manner as in Example 1-1 except that Compound Y1-5 was used as the second hole transporting layer in Example 1-1.

### Example 1-6

An organic EL device was produced in the same manner as in Example 1-1 except that Compound Y1-6 was used as the second hole transporting layer in Example 1-1.

### Comparative Example 1-1

An organic EL device was produced in the same manner as in Example 1-1 except that Compound Z1-1 (Af: 2.43 eV, Eg(S): 3.21 eV) was used as the second hole transporting layer in Example 1-1.

### Comparative Example 1-2

An organic EL device was produced in the same manner as in Example 1-1 except that Compound Z1-2 was used as the second hole transporting layer in Example 1-1.

### Comparative Example 1-3

An organic EL device was produced in the same manner as in Example 1-1 except that Compound Z1-3 was used as the second hole transporting layer in Example 1-1.

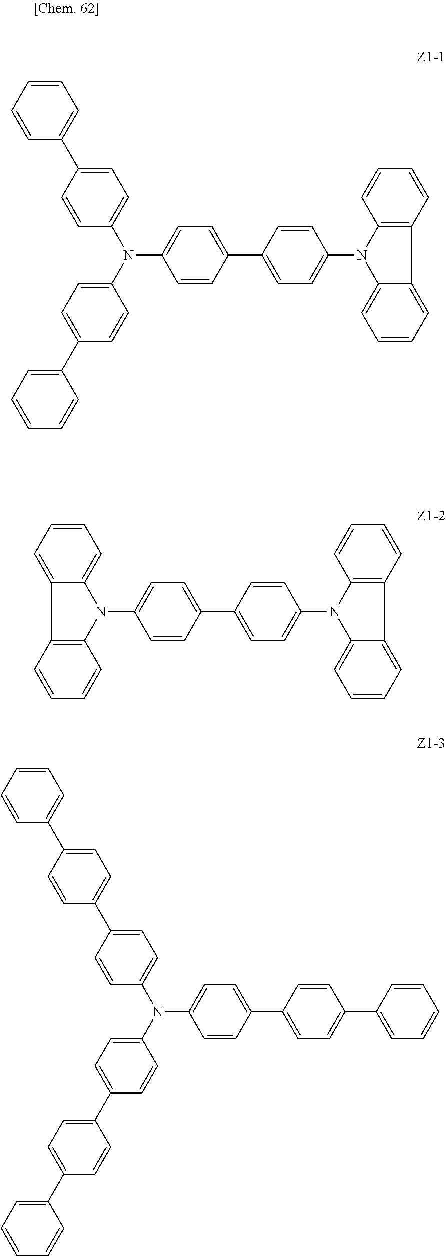

[Characteristics of Organic EL Device, Evaluation for Lifetime]

Table 1 below shows the half lifetimes of the respective organic EL devices produced as described above at an initial luminance of 20,000 cd/m².

TABLE 1

| | First hole | Second hole | Initial | Half |
|---|---|---|---|---|
| | transporting | transporting | luminance | lifetime |
| | layer | layer | (cd/m²) | (hr) |
| Example 1-1 | X1 | Y1-1 | 20,000 | 610 |
| Example 1-2 | X1 | Y1-2 | 20,000 | 600 |
| Example 1-3 | X1 | Y1-3 | 20,000 | 550 |
| Example 1-4 | X1 | Y1-4 | 20,000 | 700 |
| Example 1-5 | X1 | Y1-5 | 20,000 | 620 |
| Example 1-6 | X1 | Y1-6 | 20,000 | 700 |
| Comparative | X1 | Z1-1 | 20,000 | 100 |
| Example 1-1 | | | | |
| Comparative | X1 | Z1-2 | 20,000 | 20 |
| Example 1-2 | | | | |
| Comparative | X1 | Z1-3 | 20,000 | 290 |
| Example 1-3 | | | | |

### Example 1-7

An organic EL device was produced in the same manner as in Example 1-1 except that Compound X2 was used as the first hole transporting layer in Example 1-1.

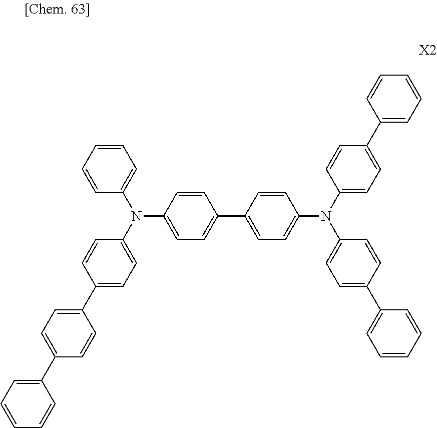

### Examples 1-8 to 1-12

Organic EL devices were each produced in the same manner as in Example 1-7 except that a material shown in Table 2 was used as the second hole transporting layer in Example 1-7.

### Comparative Example 1-4

An organic EL device was produced in the same manner as in Example 1-1 except that Compound X2 was used as the first hole transporting layer and Compound Z1-1 was used as the second hole transporting layer in Example 1-1.

### Comparative Examples 1-5 and 1-6

Organic EL devices were each produced in the same manner as in Comparative Example 1-4 except that a material shown in Table 2 was used as the second hole transporting layer in Comparative Example 1-4.

[Characteristics of Organic EL Device, Evaluation for Lifetime]

Table 2 below shows the half lifetimes of the respective organic EL devices produced as described above at an initial luminance of 20,000 cd/m².

TABLE 2

| | First hole | Second hole | Initial | Half |
|---|---|---|---|---|
| | transporting | transporting | luminance | lifetime |
| | layer | layer | (cd/m²) | (hr) |
| Example 1-7 | X2 | Y1-1 | 20,000 | 600 |
| Example 1-8 | X2 | Y1-2 | 20,000 | 580 |
| Example 1-9 | X2 | Y1-3 | 20,000 | 500 |
| Example 1-10 | X2 | Y1-4 | 20,000 | 680 |
| Example 1-11 | X2 | Y1-5 | 20,000 | 590 |
| Example 1-12 | X2 | Y1-6 | 20,000 | 690 |
| Comparative | X2 | Z1-1 | 20,000 | 50 |
| Example 1-4 | | | | |
| Comparative | X2 | Z1-2 | 20,000 | 20 |
| Example 1-5 | | | | |
| Comparative | X2 | Z1-3 | 20,000 | 30 |
| Example 1-6 | | | | |

### Example 1-13

An organic EL device was produced in the same manner as in Example 1-1 except that Compound X3 was used as the first hole transporting layer in Example 1-1.

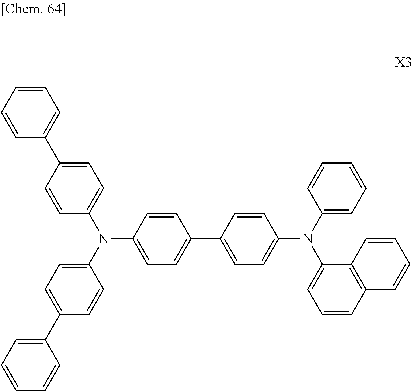

### Examples 1-14 to 1-18

Organic EL devices were each produced in the same manner as in Example 1-13 except that a material shown in Table 3 was used as the second hole transporting layer in Example 1-13.

### Comparative Example 1-7

An organic EL device was produced in the same manner as in Example 1-1 except that Compound X3 was used as the first hole transporting layer and Compound Z1-1 was used as the second hole transporting layer in Example 1-1.

### Comparative Examples 1-8 and 1-9

Organic EL devices were each produced in the same manner as in Comparative Example 1-7 except that a material shown in Table 3 was used as the second hole transporting layer in Comparative Example 1-7.

[Characteristics of Organic EL Device, Evaluation for Lifetime]

Table 3 below shows the half lifetimes of the respective organic EL devices produced as described above at an initial luminance of 20,000 cd/m².

TABLE 3

| | First hole | Second hole | Initial | Half |
|---|---|---|---|---|
| | transporting | transporting | luminance | lifetime |
| | layer | layer | (cd/m²) | (hr) |
| Example 1-13 | X3 | Y1-1 | 20,000 | 630 |
| Example 1-14 | X3 | Y1-2 | 20,000 | 620 |
| Example 1-15 | X3 | Y1-3 | 20,000 | 590 |
| Example 1-16 | X3 | Y1-4 | 20,000 | 700 |
| Example 1-17 | X3 | Y1-5 | 20,000 | 640 |
| Example 1-18 | X3 | Y1-6 | 20,000 | 700 |
| Comparative | X3 | Z1-1 | 20,000 | 130 |
| Example 1-7 | | | | |
| Comparative | X3 | Z1-2 | 20,000 | 50 |
| Example 1-8 | | | | |
| Comparative | X3 | Z1-3 | 20,000 | 320 |
| Example 1-9 | | | | |

As shown in Tables 1 to 3, the following effect was obtained. The organic EL devices of Examples 1-1 to 1-18 in each of which the first hole transporting layer and the second hole transporting layer were formed by using predetermined compounds of the present invention had increased device lifetimes as compared with those of Comparative Examples 1-1 to 1-9.

It is found that a device using Compound Y1-4 or Y1-6 as the second hole transporting layer has a longer lifetime than that of a device using Compound Y1-3.

Further, it is found that a device using Compound X1 or X3 as the first hole transporting layer has a longer lifetime than that of a device using Compound X2.

### Example 2-1

A glass substrate provided with an ITO transparent electrode measuring 25 mm by 75 mm by 1.1 mm (thickness) (manufactured by ASAHI GLASS CO., LTD.) was subjected to ultrasonic cleaning in isopropyl alcohol for 5 minutes. Then, the substrate was subjected to UV-ozone cleaning for 30 minutes.

The glass substrate provided with a transparent electrode line after the cleaning was mounted on a substrate holder of a vacuum deposition apparatus. First, Compound X1 was formed into a film having a thickness of 60 nm by resistance heating so as to serve as a first hole transporting layer and cover the transparent electrode on the surface of the glass substrate on the side where the transparent electrode line was formed.

Subsequent to the film formation of the first hole transporting layer, Compound Y1-1 was formed into a film having a thickness of 20 nm on the film by resistance heating so as to serve as a second hole transporting layer.

Further, Compound H2 as a host material and Compound D2 as a fluorescent light emitting material were co-deposited from the vapor onto the second hole transporting layer by resistance heating so as to have a thickness of 40 nm. The concentration of Compound D2 was 5%. The co-deposited film functions as a light emitting layer.

Further, subsequent to the film formation of the light emitting layer, Compound ET1 was formed into a film having a thickness of 20 nm. The ET1 film functions as an electron transporting layer.

Next, LiF was formed into a film having a thickness of 1 nm at a film formation rate of 0.1 Å/min so as to serve as an electron injectable electrode (cathode). Metal Al was deposited from the vapor onto the LiF film so that a metal cathode having a thickness of 100 nm was formed. Thus, an organic EL device was produced.

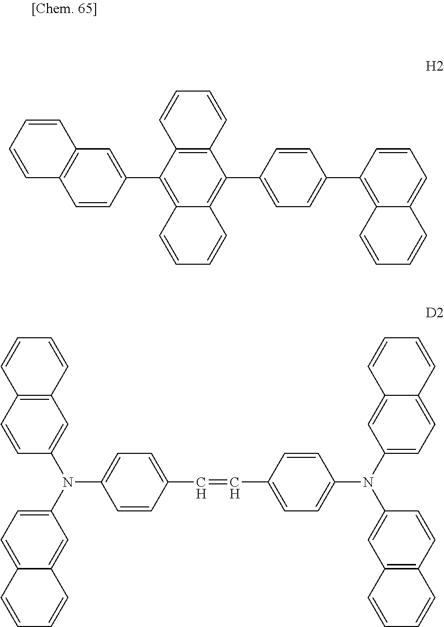

### Examples 2-2 to 2-6

Organic EL devices were each produced in the same manner as in Example 2-1 except that a material shown in Table 4 was used as the second hole transporting layer in Example 2-1.

### Comparative Example 2-1

An organic EL device was produced in the same manner as in Example 2-1 except that Compound Z1-1 was used as the second hole transporting layer in Example 2-1.

### Comparative Examples 2-2 and 2-3

Organic EL devices were each produced in the same manner as in Comparative Example 2-1 except that a material shown in Table 4 was used as the second hole transporting layer in Comparative Example 2-1.

[Characteristics of Organic EL Device, Evaluation for Lifetime]

Table 4 below shows the half lifetimes of the respective organic EL devices produced as described above at an initial luminance of 5,000 cd/m².

TABLE 4

| | First hole | Second hole | Initial | Half |
|---|---|---|---|---|
| | transporting | transporting | luminance | lifetime |
| | layer | layer | (cd/m²) | (hr) |
| Example 2-1 | X1 | Y1-1 | 5,000 | 750 |
| Example 2-2 | X1 | Y1-2 | 5,000 | 710 |
| Example 2-3 | X1 | Y1-3 | 5,000 | 620 |
| Example 2-4 | X1 | Y1-4 | 5,000 | 820 |
| Example 2-5 | X1 | Y1-5 | 5,000 | 690 |
| Example 2-6 | X1 | Y1-6 | 5,000 | 800 |
| Comparative | X1 | Z1-1 | 5,000 | 230 |
| Example 2-1 | | | | |
| Comparative | X1 | Z1-2 | 5,000 | 80 |
| Example 2-2 | | | | |
| Comparative | X1 | Z1-3 | 5,000 | 330 |
| Example 2-3 | | | | |

### Example 2-7

An organic EL device was produced in the same manner as in Example 2-1 except that Compound X2 was used as the first hole transporting layer in Example 2-1.

### Examples 2-8 to 2-12

Organic EL devices were each produced in the same manner as in Example 2-7 except that a material shown in Table 5 was used as the second hole transporting layer in Example 2-7.

### Comparative Example 2-4

An organic EL device was produced in the same manner as in Example 2-1 except that Compound X2 was used as the first hole transporting layer and Compound Z1-1 was used as the second hole transporting layer in Example 2-1.

### Comparative Examples 2-5 and 2-6

Organic EL devices were each produced in the same manner as in Comparative Example 2-4 except that a material shown in Table 5 was used as the second hole transporting layer in Comparative Example 2-4.

[Characteristics of Organic EL Device, Evaluation for Lifetime]

Table 5 below shows the half lifetimes of the respective organic EL devices produced as described above at an initial luminance of 5,000 cd/m².

TABLE 5

| | First hole | Second hole | Initial | Half |
|---|---|---|---|---|
| | transporting | transporting | luminance | lifetime |
| | layer | layer | (cd/m²) | (hr) |
| Example 2-7 | X2 | Y1-1 | 5,000 | 700 |
| Example 2-8 | X2 | Y1-2 | 5,000 | 680 |
| Example 2-9 | X2 | Y1-3 | 5,000 | 600 |
| Example 2-10 | X2 | Y1-4 | 5,000 | 770 |
| Example 2-11 | X2 | Y1-5 | 5,000 | 670 |
| Example 2-12 | X2 | Y1-6 | 5,000 | 770 |
| Comparative | X2 | Z1-1 | 5,000 | 50 |
| Example 2-4 | | | | |
| Comparative | X2 | Z1-2 | 5,000 | 50 |
| Example 2-5 | | | | |
| Comparative | X2 | Z1-3 | 5,000 | 220 |
| Example 2-6 | | | | |

### Example 2-13

An organic EL device was produced in the same manner as in Example 2-1 except that Compound X3 was used as the first hole transporting layer in Example 2-1.

### Examples 2-14 to 2-18

Organic EL devices were each produced in the same manner as in Example 2-13 except that a material shown in Table 6 was used as the second hole transporting layer in Example 2-13.

### Comparative Example 2-7

An organic EL device was produced in the same manner as in Example 2-1 except that Compound X3 was used as the first hole transporting layer and Compound Z1-1 was used as the second hole transporting layer in Example 2-1.

### Comparative Examples 2-8 and 2-9

Organic EL devices were each produced in the same manner as in Comparative Example 2-7 except that a material shown in Table 6 was used as the second hole transporting layer in Comparative Example 2-7.

[Characteristics of Organic EL Device, Evaluation for Lifetime]

Table 6 below shows the half lifetimes of the respective organic EL devices produced as described above at an initial luminance of 5,000 cd/m².

TABLE 6

| | First hole | Second hole | Initial | Half |
|---|---|---|---|---|
| | transporting | transporting | luminance | lifetime |
| | layer | layer | (cd/m²) | (hr) |
| Example 2-13 | X3 | Y1-1 | 5,000 | 760 |
| Example 2-14 | X3 | Y1-2 | 5,000 | 730 |
| Example 2-15 | X3 | Y1-3 | 5,000 | 640 |
| Example 2-16 | X3 | Y1-4 | 5,000 | 840 |
| Example 2-17 | X3 | Y1-5 | 5,000 | 720 |
| Example 2-18 | X3 | Y1-6 | 5,000 | 850 |
| Comparative | X3 | Z1-1 | 5,000 | 280 |
| Example 2-7 | | | | |
| Comparative | X3 | Z1-2 | 5,000 | 120 |
| Example 2-8 | | | | |
| Comparative | X3 | Z1-3 | 5,000 | 370 |
| Example 2-9 | | | | |

As shown in Tables 4 to 6, the following effect was obtained. The organic EL devices of Examples 2-1 to 2-18 in each of which the first hole transporting layer and the second hole transporting layer were formed by using predetermined compounds of the present invention had increased device lifetimes as compared with those of Comparative Examples 2-1 to 2-9.

It is found that a device using Compound Y1-4 or Y1-6 as the second hole transporting layer has a longer lifetime than that of a device using Compound Y1-3.

Further, it is found that a device using Compound X1 or X3 as the first hole transporting layer has a longer lifetime than that of a device using Compound X2.

### Example 3-1

A glass substrate provided with an ITO transparent electrode measuring 25 mm by 75 mm by 1.1 mm (thickness) (manufactured by ASAHI GLASS CO., LTD.) was subjected to ultrasonic cleaning in isopropyl alcohol for 5 minutes. Then, the substrate was subjected to UV-ozone cleaning for 30 minutes.

The glass substrate provided with a transparent electrode line after the cleaning was mounted on a substrate holder of a vacuum deposition apparatus. First, Compound C1 shown below was formed into a film having a thickness of 5 nm by resistance heating so as to serve as an electron acceptable substance and cover the transparent electrode on the surface of the glass substrate on the side where the transparent electrode line was formed.

Subsequent to the film formation of the electron acceptable substance, Compound X1 was formed into a film having a thickness of 35 nm on the film by resistance heating so as to serve as a first hole transporting layer.

Subsequent to the film formation of the first hole transporting layer, Compound Y1-1 was formed into a film having a thickness of 20 nm on the film by resistance heating so as to serve as a second hole transporting layer.

Further, Compound H1 as a host material and Compound D1 as a phosphorescent light emitting material were co-deposited from the vapor onto the second hole transporting layer by resistance heating so as to have a thickness of 40 nm. The concentration of Compound D1 was 7.5%. The co-deposited film functions as a light emitting layer.

Further, Compound HB was co-deposited from the vapor onto the light emitting layer by resistance heating so as to have a thickness of 10 nm. The film functions as a hole blocking layer.

Then, subsequent to the film formation of the hole blocking layer, Compound ET1 was formed into a film having a thickness of 30 nm. The ET1 film functions as an electron transporting layer.

Next, LiF was formed into a film having a thickness of 1 nm at a film formation rate of 0.1 Å/min so as to serve as an electron injectable electrode (cathode). Metal Al was deposited from the vapor onto the LiF film so that a metal cathode having a thickness of 80 nm was formed. Thus, an organic EL device was produced.

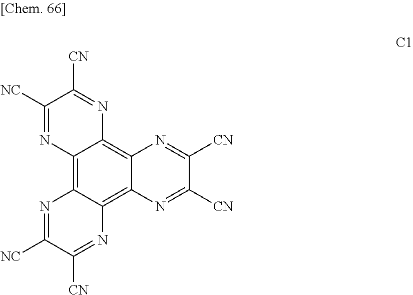

### Examples 3-2 and 3-3

Organic EL devices were each produced in the same manner as in Example 3-1 except that a material shown in Table 7 was used as the first hole transporting layer in Example 3-1.

### Example 3-4

An organic EL device was produced in the same manner as in Example 3-1 except that Compound Y1-4 was used as the second hole transporting layer in Example 3-1.

### Examples 3-5 and 3-6

Organic EL devices were each produced in the same manner as in Example 3-4 except that a material shown in Table 7 was used as the first hole transporting layer in Example 3-4.

### Example 3-7

An organic EL device was produced in the same manner as in Example 3-1 except that Compound Y1-6 was used as the second hole transporting layer in Example 3-1.

### Examples 3-8 and 3-9

Organic EL devices were each produced in the same manner as in Example 3-7 except that a material shown in Table 7 was used as the first hole transporting layer in Example 3-7.

### Comparative Example 3-1

An organic EL device was produced in the same manner as in Example 3-1 except that Compound Z1-3 was used as the second hole transporting layer in Example 3-1.

### Comparative Examples 3-2 and 3-3

Organic EL devices were each produced in the same manner as in Comparative Example 3-1 except that a material shown in Table 7 was used as the first hole transporting layer in Comparative Example 3-1.

[Characteristics of Organic EL Device, Evaluation for Lifetime]

Table 7 below shows the half lifetimes of the respective organic EL devices produced as described above at an initial luminance of 20,000 cd/m².

TABLE 7

| | First hole | Second hole | Initial | Half |
|---|---|---|---|---|
| | transporting | transporting | luminance | lifetime |
| | layer | layer | (cd/m²) | (hr) |
| Example 3-1 | X1 | Y1-1 | 20,000 | 600 |
| Example 3-2 | X2 | Y1-1 | 20,000 | 600 |
| Example 3-3 | X3 | Y1-1 | 20,000 | 620 |
| Example 3-4 | X1 | Y1-4 | 20,000 | 690 |
| Example 3-5 | X2 | Y1-4 | 20,000 | 670 |
| Example 3-6 | X3 | Y1-4 | 20,000 | 700 |
| Example 3-7 | X1 | Y1-6 | 20,000 | 690 |
| Example 3-8 | X2 | Y1-6 | 20,000 | 680 |
| Example 3-9 | X3 | Y1-6 | 20,000 | 710 |
| Comparative | X1 | Z1-3 | 20,000 | 290 |
| Example 3-1 | | | | |
| Comparative | X2 | Z1-3 | 20,000 | 30 |
| Example 3-2 | | | | |
| Comparative | X3 | Z1-3 | 20,000 | 320 |
| Example 3-3 | | | | |

### Example 4-1

A glass substrate provided with an ITO transparent electrode measuring 25 mm by 75 mm by 1.1 mm (thickness) (manufactured by ASAHI GLASS CO., LTD.) was subjected to ultrasonic cleaning in isopropyl alcohol for 5 minutes. Then, the substrate was subjected to UV-ozone cleaning for 30 minutes.

The glass substrate provided with a transparent electrode line after the cleaning was mounted on a substrate holder of a vacuum deposition apparatus. First, Compound C1 was formed into a film having a thickness of 5 nm by resistance heating so as to serve as an electron acceptable substance and cover the transparent electrode on the surface of the glass substrate on the side where the transparent electrode line was formed.

Subsequent to the film formation of the electron acceptable substance, Compound X1 was formed into a film having a thickness of 55 nm on the film by resistance heating so as to serve as a first hole transporting layer.

Subsequent to the film formation of the first hole transporting layer, Compound Y1-1 was formed into a film having a thickness of 20 nm on the film by resistance heating so as to serve as a second hole transporting layer.

Further, Compound H2 as a host material and Compound D2 as a fluorescent light emitting material were co-deposited from the vapor onto the second hole transporting layer by resistance heating so as to have a thickness of 40 nm. The concentration of Compound D2 was 5%. The co-deposited film functions as a light emitting layer.

Further, subsequent to the film formation of the light emitting layer, Compound ET1 was formed into a film having a thickness of 20 nm. The ET1 film functions as an electron transporting layer.

Next, LiF was formed into a film having a thickness of 1 nm at a film formation rate of 0.1 Å/min so as to serve as an electron injectable electrode (cathode). Metal Al was deposited from the vapor onto the LiF film so that a metal cathode having a thickness of 100 nm was formed. Thus, an organic EL device was produced.

### Examples 4-2 and 4-3

Organic EL devices were each produced in the same manner as in Example 4-1 except that a material shown in Table 8 was used as the first hole transporting layer in Example 4-1.

### Example 4-4

An organic EL device was produced in the same manner as in Example 4-1 except that Compound Y1-4 was used as the second hole transporting layer in Example 4-1.

### Examples 4-5 and 4-6

Organic EL devices were each produced in the same manner as in Example 4-4 except that a material shown in Table 8 was used as the first hole transporting layer in Example 4-4.

### Example 4-7

An organic EL device was produced in the same manner as in Example 4-1 except that Compound Y1-4 was used as the second hole transporting layer in Example 4-1.

### Examples 4-8 and 4-9

Organic EL devices were each produced in the same manner as in Example 4-7 except that a material shown in Table 8 was used as the first hole transporting layer in Example 4-7.

### Comparative Example 4-1

An organic EL device was produced in the same manner as in Example 4-1 except that Compound Z1-3 was used as the second hole transporting layer in Example 4-1.

### Comparative Examples 4-2 and 4-3

Organic EL devices were each produced in the same manner as in Comparative Example 3-1 except that a material shown in Table 8 was used as the first hole transporting layer in Comparative Example 4-1.

[Characteristics of Organic EL Device, Evaluation for Lifetime]

Table 8 below shows the half lifetimes of the respective organic EL devices produced as described above at an initial luminance of 5,000 cd/m².

TABLE 8

| | First hole | Second hole | Initial | Half |
|---|---|---|---|---|
| | transporting | transporting | luminance | lifetime |
| | layer | layer | (cd/m²) | (hr) |
| Example 4-1 | X1 | Y1-1 | 5,000 | 740 |
| Example 4-2 | X2 | Y1-1 | 5,000 | 700 |
| Example 4-3 | X3 | Y1-1 | 5,000 | 740 |
| Example 4-4 | X1 | Y1-4 | 5,000 | 820 |
| Example 4-5 | X2 | Y1-4 | 5,000 | 750 |
| Example 4-6 | X3 | Y1-4 | 5,000 | 830 |
| Example 4-7 | X1 | Y1-6 | 5,000 | 790 |
| Example 4-8 | X2 | Y1-6 | 5,000 | 770 |
| Example 4-9 | X3 | Y1-6 | 5,000 | 850 |
| Comparative | X1 | Z1-3 | 5,000 | 320 |
| Example 4-1 | | | | |
| Comparative | X2 | Z1-3 | 5,000 | 200 |
| Example 4-2 | | | | |
| Comparative | X3 | Z1-3 | 5,000 | 350 |
| Example 4-3 | | | | |

As shown in Tables 7 and 8, the following effect was obtained. The organic EL devices of Examples 3-1 to 4-9 in each of which the first hole transporting layer and the second hole transporting layer were formed by using predetermined compounds of the present invention had increased device lifetimes as compared with those of Comparative Examples 3-1 to 4-3.

It is found that a device using Compound Y1-4 or Y1-6 as the second hole transporting layer has a longer lifetime than that of a device using Compound Y1-1.

Further, it is found that a device using Compound X1 or X3 as the first hole transporting layer has a longer lifetime than that of a device using Compound X2.

### Example 5-1

A glass substrate provided with an ITO transparent electrode measuring 25 mm by 75 mm by 1.1 mm (thickness) (manufactured by ASAHI GLASS CO., LTD.) was subjected to ultrasonic cleaning in isopropyl alcohol for 5 minutes. Then, the substrate was subjected to UV-ozone cleaning for 30 minutes.

The glass substrate provided with a transparent electrode line after the cleaning was mounted on a substrate holder of a vacuum deposition apparatus. First, Compound C1 was formed into a film having a thickness of 5 nm by resistance heating so as to serve as an electron acceptable substance and cover the transparent electrode on the surface of the glass substrate on the side where the transparent electrode line was formed.

Subsequent to the film formation of the electron acceptable substance, Compound Y1-7 was formed into a film having a thickness of 55 nm on the film by resistance heating so as to serve as a first hole transporting layer.

Further, Compound H1 as a host material and Compound D1 as a phosphorescent light emitting material were co-deposited from the vapor onto the first hole transporting layer by resistance heating so as to have a thickness of 40 nm. The concentration of Compound D1 was 7.5%. The co-deposited film functions as a light emitting layer.

Further, Compound HB was co-deposited from the vapor onto the light emitting layer by resistance heating so as to have a thickness of 10 nm. The film functions as a hole blocking layer.

Then, subsequent to the film formation of the hole blocking layer, Compound ET1 was formed into a film having a thickness of 30 nm. The ET1 film functions as an electron transporting layer.

Next, LiF was formed into a film having a thickness of 1 nm at a film formation rate of 0.1 Å/min so as to serve as an electron injectable electrode (cathode). Metal Al was deposited from the vapor onto the LiF film so that a metal cathode having a thickness of 80 nm was formed. Thus, an organic EL device was produced.

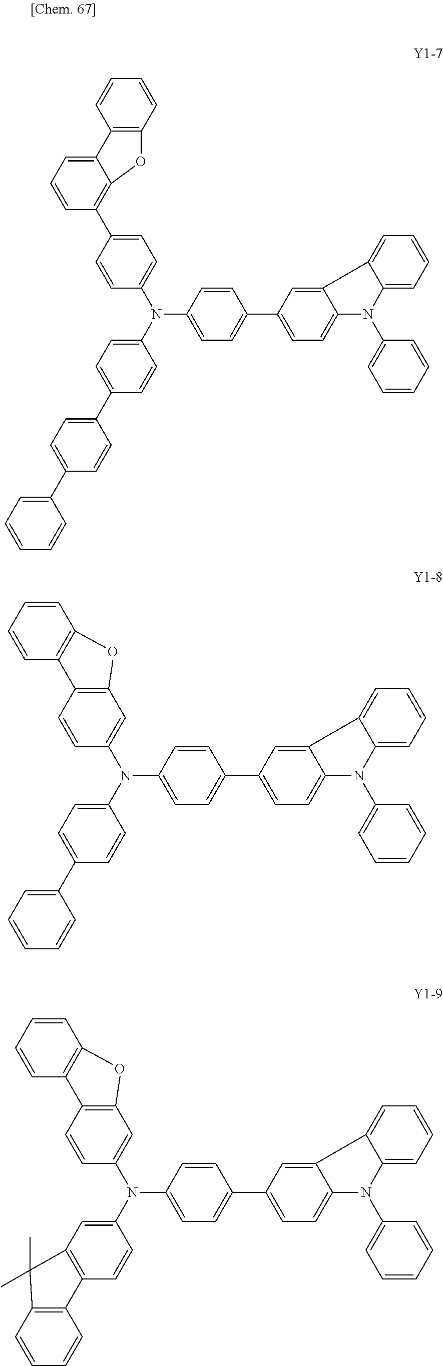

### Examples 5-2 and 5-3

Organic EL devices were each produced in the same manner as in Example 5-1 except that a material shown in Table 9 was used as the first hole transporting layer in Example 5-1.

### Comparative Example 5-1

An organic EL device was produced in the same manner as in Example 5-1 except that Compound Z1-3 was used as the first hole transporting layer in Example 5-1.

[Characteristics of Organic EL Device, Evaluation for Lifetime]

Table 9 below shows the driving voltages at 10 mA/cm² and half lifetimes at an initial luminance of 20,000 cd/m² of the respective organic EL devices produced as described above.

TABLE 9

| | First hole | Driving voltage | Half lifetime |
|---|---|---|---|
| | transporting layer | (V) | (hr) |
| Example 5-1 | Y1-7 | 4.9 | 650 |
| Example 5-2 | Y1-8 | 4.8 | 620 |
| Example 5-3 | Y1-9 | 4.7 | 620 |
| Comparative | Z1-3 | 6.0 | 330 |
| Example 5-1 | | | |

### Example 6-1

A glass substrate provided with an ITO transparent electrode measuring 25 mm by 75 mm by 1.1 mm (thickness) (manufactured by ASAHI GLASS CO., LTD.) was subjected to ultrasonic cleaning in isopropyl alcohol for 5 minutes. Then, the substrate was subjected to UV-ozone cleaning for 30 minutes.

The glass substrate provided with a transparent electrode line after the cleaning was mounted on a substrate holder of a vacuum deposition apparatus. First, Compound C1 was formed into a film having a thickness of 5 nm by resistance heating so as to serve as an electron acceptable substance and cover the transparent electrode on the surface of the glass substrate on the side where the transparent electrode line was formed.

Subsequent to the film formation of the electron acceptable substance, Compound Y1-7 was formed into a film having a thickness of 75 nm on the film by resistance heating so as to serve as a first hole transporting layer.

Further, Compound H2 as a host material and Compound D2 as a fluorescent light emitting material were co-deposited from the vapor onto the first hole transporting layer by resistance heating so as to have a thickness of 40 nm. The concentration of Compound D2 was 5%. The co-deposited film functions as a light emitting layer.

Further, subsequent to the film formation of the light emitting layer, Compound ET1 was formed into a film having a thickness of 20 nm. The ET1 film functions as an electron transporting layer.

Next, LiF was formed into a film having a thickness of 1 nm at a film formation rate of 0.1 Å/min so as to serve as an electron injectable electrode (cathode). Metal Al was deposited from the vapor onto the LiF film so that a metal cathode having a thickness of 100 nm was formed. Thus, an organic EL device was produced.

### Examples 6-2 and 6-3

Organic EL devices were each produced in the same manner as in Example 6-1 except that a material shown in Table 10 was used as the first hole transporting layer in Example 6-1.

### Comparative Example 6-1

An organic EL device was produced in the same manner as in Example 6-1 except that Compound Z1-3 was used as the first hole transporting layer in Example 6-1.

[Characteristics of Organic EL Device, Evaluation for Lifetime]

Table 10 below shows, the driving voltages at 10 mA/cm² and half lifetimes at an initial luminance of 5,000 cd/m² of the respective organic EL devices produced as described above.

TABLE 10

| | First hole | Driving voltage | Half lifetime |
|---|---|---|---|
| | transporting layer | (V) | (hr) |
| Example 6-1 | Y1-7 | 4.3 | 700 |
| Example 6-2 | Y1-8 | 4.2 | 670 |
| Example 6-3 | Y1-9 | 4.0 | 670 |
| Comparative | Z1-3 | 6.5 | 370 |
| Example 6-1 | | | |

As shown in Tables 9 and 10, the following effect was obtained. The organic EL devices of Examples 5-1 to 6-3 in each of which the electron acceptable substance and the first hole transporting layer were formed by using predetermined compounds of the present invention had reduced driving voltages and increased device lifetimes as compared with those of Comparative Examples 5-1 to 6-1.

### INDUSTRIAL APPLICABILITY

As described above in detail, the organic EL device of the present invention is extremely useful as an organic EL device having high practicality because the device has higher efficiency and a longer lifetime than those of a conventional one.

1. An organic electroluminescence device, comprising:
an anode;
a cathode; and
an organic thin-film layer comprised between the anode and the cathode,
wherein the organic thin-film layer comprises a light emitting layer comprising a host material and a light emitting material, and a hole transporting layer comprised on a side closer to the anode than the light emitting layer,
wherein the hole transporting layer comprises a first hole transporting layer and a second hole transporting layer in the stated order from the anode;
wherein the first hole transporting layer comprises a compound of formula (1) 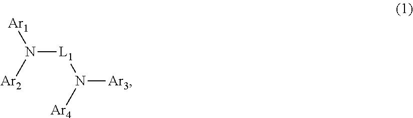
wherein
L₁ is a substituted or unsubstituted arylene group comprising 10 to 40 ring carbon atoms, and
Ar₁ to Ar₄ are each a substituted or unsubstituted aryl group comprising 6 to 60 ring carbon atoms, or a heteroaryl group 6 to 60 ring atoms; and
wherein the second hole transporting layer comprises a compound of formula (2) 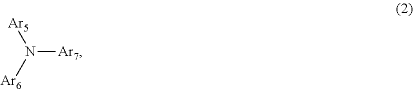
wherein, in formula (2), at least one of Ar₅ to Ar₇ is a group of formula (3) 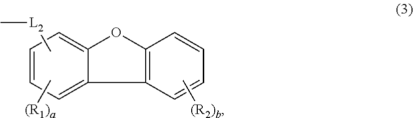
wherein
L₂ is a single bond, or a substituted or unsubstituted arylene group comprising 6 to 50 ring carbon atoms, and an optional substituent of L₂ is a linear or branched alkyl group comprising 1 to 10 carbon atoms, a cycloalkyl group comprising 3 to 10 ring carbon atoms, a trialkylsilyl group comprising 3 to 10 carbon atoms, a triarylsilyl group comprising 18 to 30 ring carbon atoms, an alkylarylsilyl group comprising 8 to 15 carbon atoms whose aryl moiety comprises 6 to 14 ring carbon atoms, an aryl group comprising 6 to 14 ring carbon atoms, a halogen atom, or a cyano group,
R₁ and R₂ are each independently a substituted or unsubstituted, linear or branched alkyl group comprising 1 to 10 carbon atoms, a substituted or unsubstituted cycloalkyl group comprising 3 to 10 ring carbon atoms, a substituted or unsubstituted trialkylsilyl group comprising 3 to 10 carbon atoms, a substituted or unsubstituted triarylsilyl group comprising 18 to 30 ring carbon atoms, a substituted or unsubstituted alkylarylsilyl group comprising 8 to 15 carbon atoms whose aryl moiety comprises 6 to 14 ring carbon atoms, a substituted or unsubstituted aryl group comprising 6 to 16 ring carbon atoms, a halogen atom, or a cyano group, and a plurality of adjacent R₁'s or R₂'s are optionally bonded to each other to form a saturated or unsaturated, divalent group that forms a ring, and
a and b are each independently an integer of 0 to 4;
wherein, in formula 2, a group of Ar₅ to Ar₇ other than the group of formula (3) is (2-i) a substituted or unsubstituted aryl group comprising 6 to 40 carbon atoms, or (2-ii) a group of formula (4) 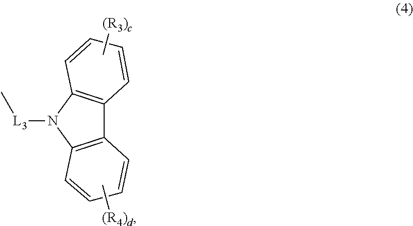
wherein:
L₃ is a single bond, or a substituted or unsubstituted arylene group comprising 6 to 50 ring carbon atoms, and an optional substituent of which L₃ is a linear or branched alkyl group comprising 1 to 10 carbon atoms, a cycloalkyl group comprising 3 to 10 ring carbon atoms, a trialkylsilyl group comprising 3 to 10 carbon atoms, a triarylsilyl group comprising 18 to 30 ring carbon atoms, an alkylarylsilyl group comprising 8 to 15 carbon atoms whose aryl moiety comprises 6 to 14 ring carbon atoms, an aryl group comprising 6 to 14 ring carbon atoms, a halogen atom, or a cyano group,
c and d are each independently an integer of 0 to 4, and
R₃ and R₄ are each independently a substituted or unsubstituted, linear or branched alkyl group comprising 1 to 10 carbon atoms, a substituted or unsubstituted cycloalkyl group comprising 3 to 10 ring carbon atoms, a substituted or unsubstituted trialkylsilyl group comprising 3 to 10 carbon atoms, a substituted or unsubstituted triarylsilyl group comprising 18 to 30 ring carbon atoms, a substituted or unsubstituted alkylarylsilyl group comprising 8 to 15 carbon atoms whose aryl moiety comprises 6 to 14 ring carbon atoms, a substituted or unsubstituted aryl group comprising 6 to 14 ring carbon atoms, a halogen atom, or a cyano group, and a plurality of adjacent R₃'s or R₄'s are optionally bonded to each other to form a saturated or unsaturated, divalent group that forms a ring;
or (2-iii) a group of formula (5) 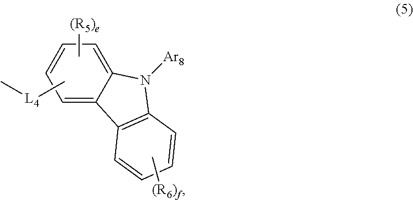
wherein
L₄ is a single bond, or a substituted or unsubstituted arylene group comprising 6 to 50 ring carbon atoms, and an optional substituent of L₄ is a linear or branched alkyl group comprising 1 to 10 carbon atoms, a cycloalkyl group comprising 3 to 10 ring carbon atoms, a trialkylsilyl group comprising 3 to 10 carbon atoms, a triarylsilyl group comprising 18 to 30 ring carbon atoms, an alkylarylsilyl group comprising 8 to 15 carbon atoms whose aryl moiety comprises 6 to 14 ring carbon atoms, an aryl group comprising 6 to 14 ring carbon atoms, a halogen atom, or a cyano group,
Ar₈ is a substituted or unsubstituted aryl group having 6 to 14 ring carbon atoms, and an optional substituent of Ar₈ is a linear or branched alkyl group comprising 1 to 10 carbon atoms, a cycloalkyl group comprising 3 to 10 ring carbon atoms, a trialkylsilyl group comprising 3 to 10 carbon atoms, a triarylsilyl group comprising 18 to 30 ring carbon atoms, an alkylarylsilyl group comprising 8 to 15 carbon atoms whose aryl moiety comprises 6 to 14 ring carbon atoms, an aryl group comprising 6 to 14 ring carbon atoms, a halogen atom, or a cyano group,
e is an integer of 0 to 3 and f represents an integer of 0 to 4, and
R₅ and R₆ are each independently a substituted or unsubstituted, linear or branched alkyl group comprising 1 to 10 carbon atoms, a substituted or unsubstituted cycloalkyl group comprising 3 to 10 ring carbon atoms, a substituted or unsubstituted trialkylsilyl group comprising 3 to 10 carbon atoms, a substituted or unsubstituted triarylsilyl group comprising 18 to 30 ring carbon atoms, a substituted or unsubstituted alkylarylsilyl group comprising 8 to 15 carbon atoms whose aryl moiety comprises 6 to 14 ring carbon atoms, a substituted or unsubstituted aryl group comprising 6 to 14 ring carbon atoms, a halogen atom, or a cyano group, and a plurality of adjacent R₅'s or R₆'s are optionally bonded to each other to form a saturated or unsaturated, divalent group that forms a ring.

2. The device of claim 1, wherein the compound of formula (1) is asymmetric with respect to L₁.

3. The device of claim 1, wherein L₁ in formula (1) is a biphenyldiyl group.

4. The device of claim 1, wherein Ar₁ to Ar₄ in formula (1) are each independently a substituted or unsubstituted phenyl group, a substituted or unsubstituted biphenylyl group, a substituted or unsubstituted terphenylyl group, or a substituted or unsubstituted phenanthryl group, or each independently have formula (6) 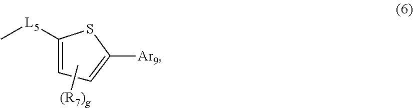
wherein
L₅ is a substituted or unsubstituted arylene group comprising 6 to 50 ring carbon atoms, and an optional substituent of L₅ is a linear or branched alkyl group comprising 1 to 10 carbon atoms, a cycloalkyl group comprising 3 to 10 ring carbon atoms, a trialkylsilyl group comprising 3 to 10 carbon atoms, a triarylsilyl group comprising 18 to 30 ring carbon atoms, an alkylarylsilyl group comprising 8 to 15 carbon atoms whose aryl moiety comprises 6 to 14 ring carbon atoms, an aryl group comprising 6 to 14 ring carbon atoms, a halogen atom, or a cyano group,
Ar₉ is a substituted or unsubstituted aryl group comprising 6 to 14 ring carbon atoms, and an optional substituent of Ar₉ is a linear or branched alkyl group comprising 1 to 10 carbon atoms, a cycloalkyl group comprising 3 to 10 ring carbon atoms, a trialkylsilyl group comprising 3 to 10 carbon atoms, a triarylsilyl group comprising 18 to 30 ring carbon atoms, an alkylarylsilyl group comprising 8 to 15 carbon atoms whose aryl moiety comprises 6 to 14 ring carbon atoms, an aryl group comprising 6 to 14 ring carbon atoms, a halogen atom, or a cyano group;
g is 1 or 2, and
R₇ is a substituted or unsubstituted, linear or branched alkyl group comprising 1 to 10 carbon atoms, a substituted or unsubstituted cycloalkyl group comprising 3 to 10 ring carbon atoms, a substituted or unsubstituted trialkylsilyl group comprising 3 to 10 carbon atoms, a substituted or unsubstituted triarylsilyl group comprising 18 to 30 ring carbon atoms, a substituted or unsubstituted alkylarylsilyl group comprising 8 to 15 carbon atoms whose aryl moiety comprises 6 to 14 ring carbon atoms, a substituted or unsubstituted aryl group comprising 6 to 14 ring carbon atoms, a halogen atom, or a cyano group, and a plurality of R₇'s are optionally bonded to each other to form a saturated or unsaturated, divalent group that forms a ring.

5. The device of claim 1, wherein Ar₁ to Ar₄ in formula (1) are each independently a substituted or unsubstituted phenyl group, a substituted or unsubstituted biphenyl group, a substituted or unsubstituted terphenyl group, or a substituted or unsubstituted phenanthryl group.

6. The device of claim 1, wherein at least one of Ar₁ to Ar₄ in formula (1) has formula (6): 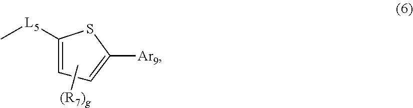
wherein
L₅ is a substituted or unsubstituted arylene group comprising 6 to 50 ring carbon atoms, and an optional substituent of L₅ is a linear or branched alkyl group comprising 1 to 10 carbon atoms, a cycloalkyl group comprising 3 to 10 ring carbon atoms, a trialkylsilyl group comprising 3 to 10 carbon atoms, a triarylsilyl group comprising 18 to 30 ring carbon atoms, an alkylarylsilyl group comprising 8 to 15 carbon atoms whose aryl moiety comprises 6 to 14 ring carbon atoms, an aryl group comprising 6 to 14 ring carbon atoms, a halogen atom, or a cyano group,
Ar₉ is a substituted or unsubstituted aryl group comprising 6 to 14 ring carbon atoms, and an optional substituent of Ar₉ is a linear or branched alkyl group comprising 1 to 10 carbon atoms, a cycloalkyl group comprising 3 to 10 ring carbon atoms, a trialkylsilyl group comprising 3 to 10 carbon atoms, a triarylsilyl group comprising 18 to 30 ring carbon atoms, an alkylarylsilyl group comprising 8 to 15 carbon atoms whose aryl moiety comprises 6 to 14 ring carbon atoms, an aryl group comprising 6 to 14 ring carbon atoms, a halogen atom, or a cyano group,
g is 1 or 2, and
R₇ is a substituted or unsubstituted, linear or branched alkyl group comprising 1 to 10 carbon atoms, a substituted or unsubstituted cycloalkyl group comprising 3 to 10 ring carbon atoms, a substituted or unsubstituted trialkylsilyl group comprising 3 to 10 carbon atoms, a substituted or unsubstituted triarylsilyl group comprising 18 to 30 ring carbon atoms, a substituted or unsubstituted alkylarylsilyl group comprising 8 to 15 carbon atoms whose aryl moiety comprises 6 to 14 ring carbon atoms, a substituted or unsubstituted aryl group comprising 6 to 14 ring carbon atoms, a halogen atom, or a cyano group, and a plurality of R₇'s are optionally bonded to each other to form a saturated or unsaturated, divalent group that forms a ring.

7. The device of claim 1, wherein at least one of Ar₅ to Ar₇ in formula (2) is a group of (4) or (5).

8. The device of claim 7, wherein at least one of Ar₅ to Ar₇ in formula (2) is a group of formula (4).

9. The device of claim 7, wherein at least one of Ar₅ to Ar₇ in formula (2) is a group of formula (5).

10. The device of claim 1, wherein at least one of Ar₅ to Ar₇ in formula (2) is of formula (7): 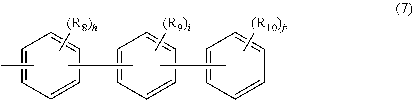
wherein
R₈ to R₁₀ are each independently a substituted or unsubstituted, linear or branched alkyl group having comprising 1 to 10 carbon atoms, a substituted or unsubstituted cycloalkyl group comprising 3 to 10 ring carbon atoms, a substituted or unsubstituted trialkylsilyl group comprising 3 to 10 carbon atoms, a substituted or unsubstituted triarylsilyl group comprising 18 to 30 ring carbon atoms, a substituted or unsubstituted alkylarylsilyl group comprising 8 to 15 carbon atoms whose aryl moiety comprises 6 to 14 ring carbon atoms, a substituted or unsubstituted aryl group comprising 6 to 16 ring carbon atoms, a halogen atom, or a cyano group, and a plurality of adjacent R₈'s, R₉'s, or R₁₀'s are optionally bonded to each other to form a saturated or unsaturated, divalent group that forms a ring, and
h, i, and j are each independently an integer of 0 to 4.

11. The device of claim 1, wherein:
at least two of Ar₅ to Ar₇ in formula (2) are of formula (3);
when two of Ar₅ to Ar₇ are of formula (3), the two groups of formula (3) are different from each other; and
when three of Ar₅ to Ar₇ are of formula (3), the three groups of formula (2) are different from one another.

12. The device of claim 1, wherein Ar₅ in of formula (2) is of formula (3).

13. The device of claim 12, wherein Ar₆ and Ar₇ in formula (2) each represent a substituted or unsubstituted aryl group comprising 6 to 40 carbon atoms.

14. The device of claim 12, wherein, in formula (2), Ar₆ is of formula (3) and Ar₇ is a substituted or unsubstituted aryl group comprising 6 to 40 carbon atoms.

15. The device of claim 12, wherein Ar₆ and Ar₇ in formula (2) are each of formula (4).

16. The device of claim 12, wherein, in formula (2), Ar₆ is of formula (3) and Ar₇ is of formula (4).

17. The device of claim 12, wherein Ar₆ and Ar₇ in formula (2) are each of formula (5).

18. The device of claim 12, wherein, in the general formula (2), Ar₆ is of formula (3) and Ar₇ is of formula (5).

19. The device of claim 12, wherein Ar₆ and Ar₇ in formula (2) are each of formula (7).

20. The device of claim 12, wherein, in formula (2), Ar₆ is of formula (3) and Ar₇ is of formula (7).

21. The device of claim 12, wherein in formula (2), Ar₆ is of formula (5).

22. The device of claim 12, wherein, in formula (2), Ar₆ is of formula (4) and Ar₇ is of formula (7).

23. The device of claim 12, wherein, in formula (2), Ar₆ is of formula (4) and Ar₇ is a substituted or unsubstituted aryl group comprising 6 to 40 carbon atoms.

24. The device of claim 12, wherein, in formula (2), Ar₆ is of formula (5) and Ar₇ is a substituted or unsubstituted aryl group comprising 6 to 40 carbon atoms.

25. The device of claim 12, wherein, in formula (2), Ar₆ is of formula (7) and Ar₇ is a substituted or unsubstituted aryl group comprising 6 to 40 carbon atoms.

26. The device of claim 1, wherein the hole transporting layer comprises a layer comprising an electron acceptable compound on a side of the first hole transporting layer close to the anode.

27. The device of claim 26, wherein the electron acceptable compound is of formula (10) 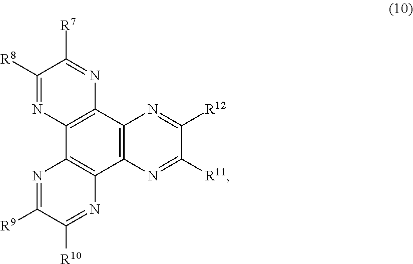
wherein
R⁷ to R¹² are each independently a cyano group, —CONH₂, a carboxyl group, or —COOR¹³, wherein R¹³ represents an alkyl group comprising 1 to 20 carbon atoms,
or
R⁷ and R⁸, R⁹ and R¹⁰, or R¹¹ and R¹² are bonded to each other to represent a group represented by —CO—O—CO—.

28. The device of claim 26, wherein the electron acceptable compound is of formula (11) 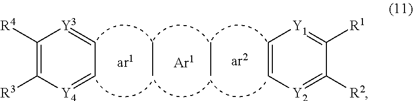
wherein Ar¹ is a fused ring comprising 6 to 24 ring carbon atoms or a heterocyclic ring comprising 6 to 24 ring atoms,
ar¹ and ar² are identical to or different from each other, and have formula (i) or (ii) 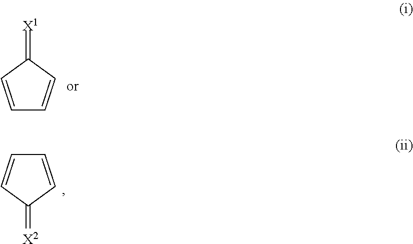
wherein X¹ and X² are identical to or different from each other, and each have a formula 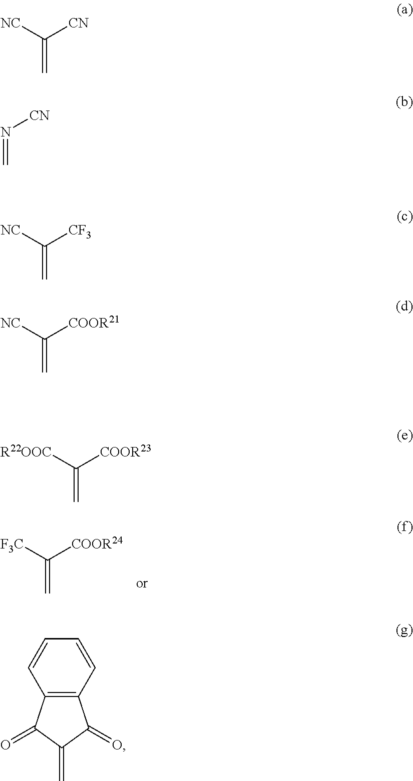
wherein R²¹ to R²⁴ are identical to or different from one another, and are a hydrogen atom, a substituted or unsubstituted fluoroalkyl group comprising 1 to 20 carbon atoms, a substituted or unsubstituted alkyl group having comprising 1 to 20 carbon atoms, a substituted or unsubstituted aryl group comprising 6 to 50 carbon atoms, or a substituted or unsubstituted heterocyclic group comprising 3 to 50 ring atoms, and R²² and R²³ are optionally bonded to each other to form a ring,
R¹ to R⁴ in formula (11) are identical to or different from one another, and are a hydrogen atom, a substituted or unsubstituted alkyl group comprising 1 to 20 carbon atoms, a substituted or unsubstituted aryl group comprising 6 to 50 carbon atoms, a substituted or unsubstituted heterocyclic group comprising 3 to 50 ring atoms, a halogen atom, a substituted or unsubstituted fluoroalkyl group comprising 1 to 20 carbon atoms, a substituted or unsubstituted alkoxy group comprising 1 to 20 carbon atoms, a substituted or unsubstituted fluoroalkoxy group comprising 1 to 20 carbon atoms, a substituted or unsubstituted aryloxy group comprising 6 to 50 carbon atoms, or a cyano group, and groups adjacent to each other out of R¹ to R⁴ are optionally bonded to each other to form a ring,
Y¹ to Y⁴ in formula (ii) are identical to or different from one another, and each are —CH═, or C(R⁵)═, and
R⁵ is a substituted or unsubstituted alkyl group comprising 1 to 20 carbon atoms, a substituted or unsubstituted aryl group comprising 6 to 50 carbon atoms, a substituted or unsubstituted heterocyclic group comprising 3 to 50 ring atoms, a halogen atom, a substituted or unsubstituted fluoroalkyl group comprising 1 to 20 carbon atoms, a substituted or unsubstituted alkoxy group comprising 1 to 20 carbon atoms, a substituted or unsubstituted fluoroalkoxy group comprising 1 to 20 carbon atoms, a substituted or unsubstituted aryloxy group comprising 6 to 50 carbon atoms, or a cyano group.

29. An organic electroluminescence device, comprising:
an anode,
a cathode; and
an organic thin-film layer comprised between the anode and the cathode,
wherein the organic thin-film layer comprises a light emitting layer comprising a host material and a light emitting material, and a hole transporting layer comprised on a side closer to the anode than the light emitting layer,
wherein the hole transporting layer comprises a layer comprising an electron acceptable compound and a first hole transporting layer in the stated order from the anode,
wherein the electron acceptable compound is of formula (10) 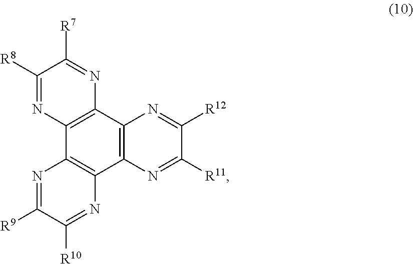
wherein
R⁷ to R¹² are each independently a cyano group, —CONH₂, a carboxyl group, or —COOR¹³ wherein R¹³ represents an alkyl group comprising 1 to 20 carbon atoms,
or
R⁷ and R⁸, R⁹ and R¹⁰, or R¹¹ and R¹² are bonded to each other to represent a group represented by —CO—O—CO—, and
wherein the first hole transporting layer comprises a compound of formula (2) 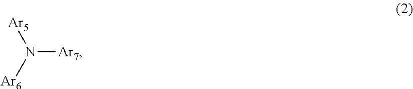
wherein at least one of Ar₅ to Ar₇ is a group of formula (3) 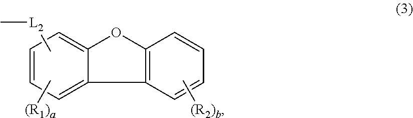
wherein
L₂ is a single bond, or a substituted or unsubstituted arylene group comprising 6 to 50 ring carbon atoms, and an optional substituent of L₂ is a linear or branched alkyl group comprising 1 to 10 carbon atoms, a cycloalkyl group comprising 3 to 10 ring carbon atoms, a trialkylsilyl group comprising 3 to 10 carbon atoms, a triarylsilyl group comprising 18 to 30 ring carbon atoms, an alkylarylsilyl group comprising 8 to 15 carbon atoms whose aryl moiety comprises 6 to 14 ring carbon atoms, an aryl group comprising 6 to 14 ring carbon atoms, a halogen atom, or a cyano group, and
R₁ and R₂ are each independently a substituted or unsubstituted, linear or branched alkyl group comprising 1 to 10 carbon atoms, a substituted or unsubstituted cycloalkyl group comprising 3 to 10 ring carbon atoms, a substituted or unsubstituted trialkylsilyl group comprising 3 to 10 carbon atoms, a substituted or unsubstituted triarylsilyl group comprising 18 to 30 ring carbon atoms, a substituted or unsubstituted alkylarylsilyl group comprising 8 to 15 carbon atoms whose aryl moiety comprises 6 to 14 ring carbon atoms, a substituted or unsubstituted aryl group having comprising 6 to 16 ring carbon atoms, a halogen atom, or a cyano group, and a plurality of adjacent R₁'s or R₂'s are optionally bonded to each other to form a saturated or unsaturated, divalent group that forms a ring, and
a and b are each independently an integer of 0 to 4,
wherein, in formula (2), any one of Ar₅ to Ar₇ other than the group of formula (3) is (2-i) a substituted or unsubstituted aryl group comprising 6 to 40 carbon atoms,
or (2-ii) a group of formula (4) 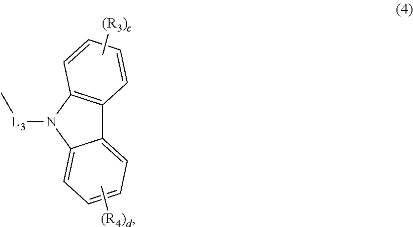
wherein
L₃ is a single bond, or a substituted or unsubstituted arylene group comprising 6 to 50 ring carbon atoms, and an optional substituent of L₃ is a linear or branched alkyl group comprising 1 to 10 carbon atoms, a cycloalkyl group having 3 to 10 ring carbon atoms, a trialkylsilyl group comprising 3 to 10 carbon atoms, a triarylsilyl group comprising 18 to 30 ring carbon atoms, an alkylarylsilyl group comprising 8 to 15 carbon atoms whose aryl moiety comprises 6 to 14 ring carbon atoms, an aryl group comprising 6 to 14 ring carbon atoms, a halogen atom, or a cyano group,
c and d are each independently an integer of 0 to 4, and
R₃ and R₄ are each independently a substituted or unsubstituted, linear or branched alkyl group comprising 1 to 10 carbon atoms, a substituted or unsubstituted cycloalkyl group comprising 3 to 10 ring carbon atoms, a substituted or unsubstituted trialkylsilyl group comprising 3 to 10 carbon atoms, a substituted or unsubstituted triarylsilyl group comprising 18 to 30 ring carbon atoms, a substituted or unsubstituted alkylarylsilyl group comprising 8 to 15 carbon atoms whose aryl moiety comprises 6 to 14 ring carbon atoms, a substituted or unsubstituted aryl group comprising 6 to 14 ring carbon atoms, a halogen atom, or a cyano group, and a plurality of adjacent R₃'s or R₄'s are optionally bonded to each other to form a saturated or unsaturated, divalent group that forms a ring
or (2-iii) a group of formula (5) 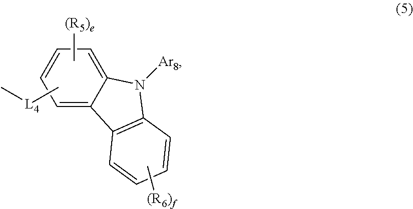
wherein
L₄ is a single bond, or a substituted or unsubstituted arylene group comprising 6 to 50 ring carbon atoms, and an optional substituent of L₄ is a linear or branched alkyl group comprising 1 to 10 carbon atoms, a cycloalkyl group comprising 3 to 10 ring carbon atoms, a trialkylsilyl group comprising 3 to 10 carbon atoms, a triarylsilyl group comprising 18 to 30 ring carbon atoms, an alkylarylsilyl group comprising 8 to 15 carbon atoms whose aryl has comprises 6 to 14 ring carbon atoms, an aryl group comprising 6 to 14 ring carbon atoms, a halogen atom, or a cyano group,
Ar₈ is a substituted or unsubstituted aryl group comprising 6 to 14 ring carbon atoms, and an optional substituent of Ar₈ is a linear or branched alkyl group comprising 1 to 10 carbon atoms, a cycloalkyl group comprising 3 to 10 ring carbon atoms, a trialkylsilyl group comprising 3 to 10 carbon atoms, a triarylsilyl group comprising 18 to 30 ring carbon atoms, an alkylarylsilyl group comprising 8 to 15 carbon atoms whose aryl moiety comprises 6 to 14 ring carbon atoms, an aryl group comprising 6 to 14 ring carbon atoms, a halogen atom, or a cyano group,
e is an integer of 0 to 3,
f is an integer of 0 to 4, and
R₅ and R₆ are each independently a substituted or unsubstituted, linear or branched alkyl group comprising 1 to 10 carbon atoms, a substituted or unsubstituted cycloalkyl group comprising 3 to 10 ring carbon atoms, a substituted or unsubstituted trialkylsilyl group comprising 3 to 10 carbon atoms, a substituted or unsubstituted triarylsilyl group comprising 18 to 30 ring carbon atoms, a substituted or unsubstituted alkylarylsilyl group comprising 8 to 15 carbon atoms whose aryl moiety comprises 6 to 14 ring carbon atoms, a substituted or unsubstituted aryl group comprising 6 to 14 ring carbon atoms, a halogen atom, or a cyano group, and a plurality of adjacent R₅'s or R₆'s are optionally bonded to each other to form a saturated or unsaturated, divalent group that forms a ring.

30. The device of claim 1, wherein the hole transporting layer is joined to the light emitting layer.

31. The device of claim 1, wherein the light emitting material comprises a metal complex compound comprising at least one metal selected from the group consisting of Ir, Pt, Os, Cu, Ru, Re, and Au.

32. The device of claim 1, wherein in the light emitting material, a central metal atom, and a carbon atom in a ligand are bonded through an ortho-metal bond.

33. The device of claim 1, wherein the host material has an excited triplet energy gap of 2.0 eV or more and 3.2 eV or less.

34. The device of claim 1, further comprising:
a reduction-causing dopant added to an interfacial region between the cathode and the organic thin-film layer.

35. The device of claim 1, further comprising:
an electron injecting layer between the light emitting layer and the cathode,
wherein the electron injecting layer comprises a nitrogen-comprising ring derivative as a main component.
